(19) United States
(12) Patent Application Publication
(10) Pub. No.: US 2020/0369630 A1
(43) Pub. Date: Nov. 26, 2020

(54) HERBICIDALLY ACTIVE 3-PHENYL-5-TRIFLUOROMETHYLISOXAZOLINE-5-CARBOXAMIDES OF CYCLOPENTYLCARBOXYLIC ACIDS AND ESTERS

(71) Applicant: Bayer Aktiengesellschaft, Leverkusen (DE)

(72) Inventors: Stephen David LINDELL, Eppstein (DE); Klaus Bernhard HAAF, Kelkheim (DE); Andreas VAN ALMSICK, Karben (DE); Hansjoerg DIETRICH, Liederbach am Taunus (DE); Anu Bheemaiah MACHETTIRA, Frankfurt am Main (DE); Christopher Hugh ROSINGER, Hofheim (DE); Elmar GATZWEILER, Bad Nauheim (DE); Elisabeth ASMUS, Hoesbach (DE)

(21) Appl. No.: 16/638,884

(22) PCT Filed: Aug. 13, 2018
(86) PCT No.: PCT/EP2018/071910

§ 371 Date: Feb. 13, 2020

(30) Foreign Application Priority Data
Aug. 17, 2017 (EP) 17186709.6

(51) Int. Cl. C07D 261/04; A01N 43/80

(52) U.S. Cl. CPC C07D 261/04; A01N 43/80

(57) ABSTRACT

The invention relates to 3-phenyl-5-trifluoromethylisoxazoline-5-carboxamides of cyclopentylcarboxylic acids and esters of the general formula (I) and agrochemically acceptable salts thereof

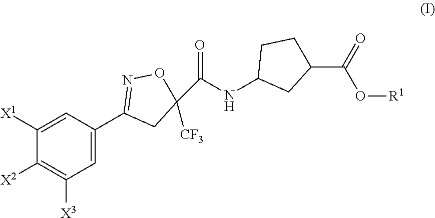

and to their use in the field of crop protection.

The invention relates to the technical field of the herbicides, especially that of the herbicides for selective control of weeds and weed grasses in crops of useful plants.

Specifically, it relates to substituted 3-phenyl-5-trifluoromethylisoxazoline-5-carboxamides of cyclopentylcarboxylic acids and esters, to processes for their preparation and to their use as herbicides.

WO1995/014681 A1, WO1995/014680 A1, WO 2008/035315 A1, WO2005/051931 A1 and WO2005/021515 A1 each describe, inter alia, 3-phenylisoxazoline-5-carboxamides which are substituted at the phenyl ring in the 3- and 4-positions by alkoxy radicals. WO1998/057937 A1 describes, inter alia, compounds which are substituted at the phenyl ring in the 4-position by an alkoxy radical. WO2006/016237 A1 describes, inter alia, in each case those compounds which are substituted at the phenyl ring by an amido radical. The compounds described in the documents mentioned above are disclosed in these documents as being pharmacologically active.

WO2005/021516 A1 discloses 3-(([3-(3-tert-butylphenyl)-5-ethyl-4,5-dihydro-1,2-oxazol-5-yl]carbonyl)amino)-5-fluoro-4-oxopentanoic acid and 3-(([3-(3-tert-butylphenyl)-5-isopropyl-4,5-dihydro-1,2-oxazol-5-yl]carbonyl)amino)-5-fluoro-4-oxopentanoic acid as pharmacologically active compounds.

DE 4026018 A1, EP 0 520 371 A2 and DE 4017665 disclose 3-phenylisoxazoline-5-carboxamides bearing a hydrogen atom in the 5 position of the isoxazoline ring. These compounds are described therein as agrochemically active safeners, i.e. as compounds which eliminate the unwanted herbicidal action of herbicides on crop plants. No herbicidal action of these compounds is disclosed. European patent application No. 10170238, which has an earlier priority date but was yet to be published at the priority date of the present application, discloses herbicidally and fungicidally active 3-phenylisoxazoline-5-carboxamides and 3-phenylisoxazoline-5-thioamides bearing a hydrogen atom in the 5 position of the isoxazoline ring. Monatshefte Chemie (2010) 141, 461 and Letters in Organic Chemistry (2010), 7, 502 also disclose 3-phenylisoxazoline-5-carboxamides bearing a hydrogen atom in the 5 position of the isoxazoline ring. Fungicidal action, but not herbicidal action, is disclosed for some of the compounds mentioned.

WO 2014/048827 describes the herbicidal action of 3-phenylisoxazoline-5-carboxylic acids, -5-carboxylic esters, -5-carbaldehydes and -5-nitriles.

WO 2014/048853 discloses isoxazoline-5-carboxamides and -5-thioamides having heterocycles in the 3-position which have herbicidal and fungicidal action.

WO 2014/048940 discloses fungicidally active isoxazolinecarboxamides having a quinoline as specific heterocycle in the 3-position.

WO 2014/048882 discloses isoxazolinecarboxamides having alkoxy as specific radical in the 5-position.

WO 2012/130798 describes herbicidally and fungicidally active 3-phenylisoxazoline-5-carboxamides and -5-thioamides of substituted cycloalkyls. Disclosed are inter alia 3-phenyl-5-trifluoromethylisoxazoline-5-carboxamides. However, corresponding cyclopentylcarboxylic acids and esters are only disclosed in a generic manner.

The herbicidal activity of these known compounds, in particular at low application rates, and/or their compatibility with crop plants remain in need of improvement.

For the reasons stated, there is still a need for potent herbicides and/or plant growth regulators for the selective use in crop plants or the use on non-crop land, where these active ingredients preferably should have further advantageous properties in application, for example an improved compatibility with crop plants.

Accordingly, it is an object of the present invention to provide compounds having herbicidal activity (herbicides) which are highly effective against economically important harmful plants even at relatively low application rates and can be used selectively in crop plants, preferably with good activity against harmful plants, and at the same time preferably have good compatibility with crop plants. Preferably, these herbicidal compounds should be particularly effective and efficient against a broad spectrum of weed grasses and preferably also have good activity against a large number of weeds.

In addition to a herbicidal action, numerous compound of the formula (I) also have fungicidal action which, however, is not very pronounced.

Surprisingly, it has now been found that the 3-phenyl-5-trifluoromethylisoxazoline-5-carboxamides of cyclopentylcarboxylic acids and esters of the formula (I) defined below and their salts have excellent herbicidal activity against a broad spectrum of economically important mono- and dicotyledonous annual harmful plants.

The present invention therefore provides compounds of the general formula (I)

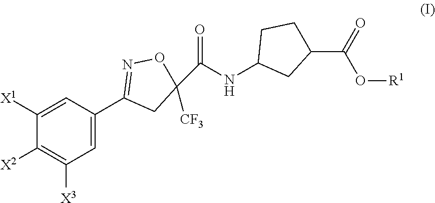

or an agrochemically acceptable salt thereof, in which
R¹ represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, bromine, cyano and (C₁-C₂)-alkoxy;
X¹ and X³ independently of one another represent hydrogen, halogen, hydroxy, cyano, nitro, S(O)ₙR² or CO₂R³,
   or
   represent (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of halogen and cyano,
   where X¹ and X³ do not simultaneously represent hydrogen;
X² represents hydrogen, halogen, cyano, nitro, S(O)ₙR² or CO₂R³,
   or
   represents (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of halogen and cyano;
R² represents (C₁-C₆)-alkyl or (C₃-C₆)-cycloalkyl, each of which is substituted by m radicals from the group consisting of halogen and cyano;
R³ represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, bromine, cyano and (C₁-C₂)-alkoxy;
m represents the running number 0, 1, 2, 3, 4 or 5; and
n represents the running number 0, 1 or 2.

Alkyl means saturated straight-chain or branched hydrocarbyl radicals having the number of carbon atoms specified in each case, e.g. C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Halogen-substituted alkyl means straight-chain or branched alkyl groups where some or all of the hydrogen atoms in these groups may be replaced by halogen atoms, e.g. C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro, 2-difluoroethyl, 2,2-dichloro-2-fluoroethyl, 2,2,2-trichloroethyl, pentafluoroethyl and 1,1,1-trifluoroprop-2-yl.

Alkenyl represents unsaturated straight-chain or branched hydrocarbyl radicals having the number of carbon atoms stated in each case and one double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl means straight-chain or branched hydrocarbyl radicals having the number of carbon atoms specified in each case and one triple bond in any position, e.g. C₂-C₆-alkynyl such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Cycloalkyl means a carbocyclic saturated ring system having preferably 3-6 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene.

Alkoxy means saturated straight-chain or branched alkoxy radicals having the number of carbon atoms specified in each case, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Halogen-substituted alkoxy means straight-chain or branched alkoxy radicals having the number of carbon atoms specified in each case, where some or all of the hydrogen atoms in these groups may be replaced by halogen atoms as specified above, e.g. C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-1,2-difluoroethoxy, 2,2-dichloro-2-fluoroethoxy, 2,2,2-trichloroethoxy, pentafluoroethoxy and 1,1,1-trifluoroprop-2-oxy.

The term “halogen” means fluorine, chlorine, bromine or iodine. If the term is used for a radical, “halogen” means a fluorine, chlorine, bromine or iodine atom.

The compounds of the formula (I) are present as stereoisomers. These stereoisomers are, for example, enantiomers or diastereomers. Stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries.

The invention also relates to all stereoisomers and mixtures thereof which are encompassed by the formula (I) but not defined specifically. However, the following text will, for the sake of simplicity, always mention compounds of the formula (I), even though this is understood as meaning not only the pure compounds, but also, if appropriate, mixtures with various amounts of isomeric compounds.

According to the nature of the substituents defined above, the compounds of the formula (I) have acidic properties and can form salts, and if appropriate also internal salts or adducts with inorganic or organic bases or with metal ions. If the compounds of the formula (I) carry hydroxyl, carboxyl or other groups which induce acidic properties, these compounds can be reacted with bases to give salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and chlorocholine, and also organic amines such as trialkylamines, morpholine, piperidine or pyridine. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NRR′R″R′″]⁺ in which R to R′″ each independently of one another represent an organic radical, in particular alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

If a group is poly substituted by radicals, this means that this group is substituted by one or more identical or different radicals from those mentioned.

In all the formulae specified hereinafter, the substituents and symbols have the same meaning as described in formula (I), unless defined differently. Arrows in a chemical formula denote the points at which it is joined to the rest of the molecule.

There follows a description of preferred, particularly preferred and very particularly preferred definitions of each of the individual substituents. The other substituents of the general formula (I) which are not specified hereinafter have the definition given above.

According to a first embodiment of the present invention,
R¹ preferably represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy;
   particularly preferably
R¹ represents hydrogen,
   or
   represents (C₁-C₆)-alkyl which is in each case substituted by m radicals from the group consisting of fluorine, chlorine, cyano and (C₁-C₂)-alkoxy;
   and very particularly preferably
R¹ represents hydrogen,
   or
   represents (C₁-C₄)-alkyl which is in each case substituted by m radicals from the group consisting of fluorine, chlorine, cyano and (C₁-C₂)-alkoxy.

According to a second embodiment of the present invention,
X¹ and X³ preferably represent hydrogen, halogen, cyano, nitro, S(O)ₙR² or CO₂R³,
   or
   represent (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of fluorine, chlorine and cyano, where X¹ and X³ do not both simultaneously represent hydrogen;
   particularly preferably)
X¹ and X³ independently of one another represent hydrogen, fluorine, chlorine or cyano,
   or
   represent (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of fluorine, chlorine and cyano, where X¹ and X³ do not both simultaneously represent hydrogen;
   very particularly preferably)
X¹ and X³ independently of one another represent hydrogen, fluorine, chlorine or cyano,
   or
   represent (C₁-C₆)-alkyl or (C₁-C₆)-alkoxy, each of which is substituted by m radicals from the group consisting of fluorine, chlorine and cyano, where X¹ and X³ do not both simultaneously represent hydrogen;
   and most preferably)
X¹ and X³ independently of one another represent hydrogen, fluorine, chlorine or cyano,
   or
   represent methyl or methoxy, each of which is substituted by m radicals from the group consisting of fluorine and chlorine, where X¹ and X³ do not both simultaneously represent hydrogen.

According to a third embodiment of the present invention,
X² preferably represents hydrogen or halogen, particularly preferably hydrogen or fluorine and most preferably hydrogen.

According to a fourth embodiment of the present invention,
R² preferably represents (C₁-C₆)-alkyl or (C₃-C₆)-cycloalkyl, each of which is substituted by m radicals from the group consisting of fluorine, chlorine and cyano.

According to a fifth embodiment of the present invention,
R³ preferably represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy.

According to a sixth embodiment of the present invention,

m represents the running number 0, 1, 2, 3 or 4 and particularly preferably 0, 1, 2 or 3.

According to a seventh embodiment of the present invention,

n represents the running number 0 or 2.

In the context of the present invention, the individual preferred and particularly preferred meanings of the substituents R¹ to R³, X¹ to X³, and the running numbers m and n can be combined with one another as desired.

This means that the present invention encompasses compounds of the general formula (I) in which, for example, the substituent R¹ has a preferred meaning and the substituents X¹ to R³ have the general definition or else the substituent X² has a preferred meaning, the substituent R¹ has a particularly preferred meaning and the remaining substituents have a general meaning.

Three of these combinations of the definitions given above for the substituents R¹ to R³, X¹ to X³, and the running numbers m and n are illustrated below by way of example, and each of them is disclosed as a further embodiment:

According to an eighth embodiment of the present invention,
R¹ represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy;
X¹ and X³ independently of one another represent hydrogen, halogen, cyano, nitro, S(O)ₙR² or CO₂R³,
   or
   represent (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of fluorine, chlorine and cyano, where X¹ and X³ do not both simultaneously represent hydrogen;
X² represents hydrogen or halogen;
R² represents (C₁-C₆)-alkyl or (C₃-C₆)-cycloalkyl, each of which is substituted by m radicals from the group consisting of fluorine, chlorine and cyano;
R³ represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy;
m represents the running number 0, 1, 2 or 3; and
n represents the running number 0 or 2.

According to a ninth embodiment of the present invention,
R¹ represents hydrogen,
   or
   represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy;
X¹ and X³ independently of one another represent hydrogen, fluorine or chlorine,
   or
   represent (C₁-C₆)-alkyl or (C₁-C₆)-alkoxy, each of which is substituted by m radicals from the group consisting of fluorine, chlorine and cyano, where X¹ and X³ do not both simultaneously represent hydrogen;
X² represents hydrogen; and
m represents the running number 0, 1, 2 or 3.

According to a tenth embodiment of the present invention,
R¹ represents hydrogen,
   or
   represents (C₁-C₄)-alkyl which is in each case substituted by m radicals from the group consisting of fluorine, chlorine, cyano and (C₁-C₂)-alkoxy;
X¹ and X³ independently of one another represent hydrogen, fluorine or chlorine, where X¹ and X³ do not simultaneously represent hydrogen;
X² represents hydrogen; and
m represents the running number 0, 1, 2 or 3.

Examples of the compounds of the general formula (I) are shown below in tabular form. Table 1 below specifies the substituents defined in general terms in formula (I).

TABLE 1
Compounds of the general formula (I)

| Example | | | | | Isoxazoline | Cyclopentyl |
|---|---|---|---|---|---|---|
| No.: | X¹ | X² | X³ | R¹ | configuration | configuration |
| I-1 | F | H | F | H | 5(rac) | cis-(rac) |
| I-2 | F | H | F | H | 5(rac) | trans-(rac) |
| I-3 | F | H | F | H | 5(rac) | 1R,3S (cis) |
| I-4 | F | H | F | H | 5(rac) | 1S,3R (cis) |
| I-5 | F | H | F | H | 5(rac) | 1R,3R (trans) |
| I-6 | F | H | F | H | 5(rac) | 1S,3S (trans) |
| I-7 | F | H | F | H | 5S | 1R,3S (cis) |
| I-8 | F | H | F | H | 5S | 1S,3R (cis) |
| I-9 | F | H | F | H | 5S | 1R,3R (trans) |
| I-10 | F | H | F | H | 5S | 1S,3S (trans) |
| I-11 | F | H | F | H | 5R | 1R,3S(cis) |
| I-12 | F | H | F | H | 5R | 1S,3R (cis) |
| I-13 | F | H | F | H | 5R | 1R,3R (trans) |
| I-14 | F | H | F | H | 5R | 1S,3S (trans) |
| I-15 | F | H | F | Me | 5(rac) | cis-(rac) |
| I-16 | F | H | F | Me | 5(rac) | trans-(rac) |
| I-17 | F | H | F | Me | 5(rac) | 1R,3S (cis) |
| I-18 | F | H | F | Me | 5(rac) | 1S,3R (cis) |
| I-19 | F | H | F | Me | 5(rac) | 1R,3R (trans) |
| I-20 | F | H | F | Me | 5(rac) | 1S,3S (trans) |
| I-21 | F | H | F | Me | 5S | 1R,3S (cis) |
| I-22 | F | H | F | Me | 5S | 1S,3R (cis) |
| I-23 | F | H | F | Me | 5S | 1R,3R (trans) |
| I-24 | F | H | F | Me | 5S | 1S,3S (trans) |
| I-25 | F | H | F | Me | 5R | 1R,3S (cis) |
| I-26 | F | H | F | Me | 5R | 1S,3R (cis) |
| I-27 | F | H | F | Me | 5R | 1R,3R (trans) |
| I-28 | F | H | F | Me | 5R | 1S,3S (trans) |
| I-29 | F | H | F | Et | 5(rac) | cis-(rac) |
| I-30 | F | H | F | Et | 5(rac) | trans-(rac) |
| I-31 | F | H | F | Et | 5(rac) | 1R,3S (cis) |
| I-32 | F | H | F | Et | 5(rac) | 1S,3R (cis) |
| I-33 | F | H | F | Et | 5(rac) | 1R,3R (trans) |
| I-34 | F | H | F | Et | 5(rac) | 1S,3S (trans) |
| I-35 | F | H | F | Et | 5S | 1R,3S (cis) |
| I-36 | F | H | F | Et | 5S | 1S,3R (cis) |
| I-37 | F | H | F | Et | 5S | 1R,3R (trans) |
| I-38 | F | H | F | Et | 5S | 1S,3S (trans) |
| I-39 | F | H | F | Et | 5R | 1R,3S (cis) |
| I-40 | F | H | F | Et | 5R | 1S,3R (cis) |
| I-41 | F | H | F | Et | 5R | 1R,3R (trans) |
| I-42 | F | H | F | Et | 5R | 1S,3S (trans) |
| I-43 | F | H | F | nPr | 5(rac) | cis-(rac) |
| I-44 | F | H | F | nPr | 5(rac) | trans-(rac) |
| I-45 | F | H | F | nPr | 5(rac) | 1R,3S (cis) |
| I-46 | F | H | F | nPr | 5(rac) | 1S,3R (cis) |
| I-47 | F | H | F | nPr | 5(rac) | 1R,3R (trans) |
| I-48 | F | H | F | nPr | 5(rac) | 1S,3S (trans) |
| I-49 | F | H | F | nPr | 5S | 1R,3S (cis) |
| I-50 | F | H | F | nPr | 5S | 1S,3R (cis) |
| I-51 | F | H | F | nPr | 5S | 1R,3R (trans) |
| I-52 | F | H | F | nPr | 5S | 1S,3S (trans) |
| I-53 | F | H | F | nPr | 5R | 1R,3S (cis) |
| I-54 | F | H | F | nPr | 5R | 1S,3R (cis) |
| I-55 | F | H | F | nPr | 5R | 1R,3R (trans) |
| I-56 | F | H | F | nPr | 5R | 1S,3S (trans) |
| I-57 | F | H | F | iPr | 5R/S | cis-R/S |
| I-58 | F | H | F | iPr | 5R/S | trans-R/S |
| I-59 | F | H | F | iPr | 5R/S | 1R,3S (cis) |
| I-60 | F | H | F | iPr | 5R/S | 1S,3R (cis) |
| I-61 | F | H | F | iPr | 5R/S | 1R,3R (trans) |
| I-62 | F | H | F | iPr | 5R/S | 1S,3S (trans) |
| I-63 | F | H | F | iPr | 5S | 1R,3S (cis) |
| I-64 | F | H | F | iPr | 5S | 1S,3R (cis) |
| I-65 | F | H | F | iPr | 5S | 1R,3R (trans) |
| I-66 | F | H | F | iPr | 5S | 1S,3S (trans) |
| I-67 | F | H | F | iPr | 5R | 1R,3S (cis) |
| I-68 | F | H | F | iPr | 5R | 1S,3R (cis) |
| I-69 | F | H | F | iPr | 5R | 1R,3R (trans) |
| I-70 | F | H | F | iPr | 5R | 1S,3S (trans) |
| I-71 | F | H | F | nBu | 5(rac) | cis-(rac) |
| I-72 | F | H | F | nBu | 5(rac) | trans-(rac) |
| I-73 | F | H | F | nBu | 5(rac) | 1R,3S (cis) |
| I-74 | F | H | F | nBu | 5(rac) | 1S,3R (cis) |
| I-75 | F | H | F | nBu | 5(rac) | 1R,3R (trans) |
| I-76 | F | H | F | nBu | 5(rac) | 1S,3S (trans) |
| I-77 | F | H | F | nBu | 5S | 1R,3S (cis) |
| I-78 | F | H | F | nBu | 5S | 1S,3R (cis) |
| I-79 | F | H | F | nBu | 5S | 1R,3R (trans) |
| I-80 | F | H | F | nBu | 5S | 1S,3S (trans) |
| I-81 | F | H | F | nBu | 5R | 1R,3S (cis) |
| I-82 | F | H | F | nBu | 5R | 1S,3R (cis) |
| I-83 | F | H | F | nBu | 5R | 1R,3R (trans) |
| I-84 | F | H | F | nBu | 5R | 1S,3S (trans) |
| I-85 | F | H | F | tBu | 5(rac) | cis-(rac) |
| I-86 | F | H | F | tBu | 5(rac) | trans-(rac) |
| I-87 | F | H | F | tBu | 5(rac) | 1R,3S (cis) |
| I-88 | F | H | F | tBu | 5(rac) | 1S,3R (cis) |
| I-89 | F | H | F | tBu | 5(rac) | 1R,3R (trans) |
| I-90 | F | H | F | tBu | 5(rac) | 1S,3S (trans) |
| I-91 | F | H | F | tBu | 5S | 1R,3S (cis) |
| I-92 | F | H | F | tBu | 5S | 1S,3R (cis) |
| I-93 | F | H | F | tBu | 5S | 1R,3R (trans) |
| I-94 | F | H | F | tBu | 5S | 1S,3S (trans) |
| I-95 | F | H | F | tBu | 5R | 1R,3S (cis) |
| I-96 | F | H | F | tBu | 5R | 1S,3R (cis) |
| I-97 | F | H | F | tBu | 5R | 1R,3R (trans) |
| I-98 | F | H | F | tBu | 5R | 1S,3S (trans) |
| I-99 | F | H | F | CH₂CH₂Cl | 5(rac) | cis-(rac) |
| I-100 | F | H | F | CH₂CH₂Cl | 5(rac) | trans-(rac) |
| I-101 | F | H | F | CH₂CH₂Cl | 5(rac) | 1R,3S (cis) |
| I-102 | F | H | F | CH₂CH₂Cl | 5(rac) | 1S,3R (cis) |
| I-103 | F | H | F | CH₂CH₂Cl | 5(rac) | 1R,3R (trans) |
| I-104 | F | H | F | CH₂CH₂Cl | 5(rac) | 1S,3S (trans) |
| I-105 | F | H | F | CH₂CH₂Cl | 5S | 1R,3S (cis) |
| I-106 | F | H | F | CH₂CH₂Cl | 5S | 1S,3R (cis) |
| I-107 | F | H | F | CH₂CH₂Cl | 5S | 1R,3R (trans) |
| I-108 | F | H | F | CH₂CH₂Cl | 5S | 1S,3S (trans) |
| I-109 | F | H | F | CH₂CH₂Cl | 5R | 1R,3S (cis) |
| I-110 | F | H | F | CH₂CH₂Cl | 5R | 1S,3R (cis) |
| I-111 | F | H | F | CH₂CH₂Cl | 5R | 1R,3R (trans) |
| I-112 | F | H | F | CH₂CH₂Cl | 5R | 1S,3S (trans) |
| I-113 | F | H | F | CH₂CF₃ | 5(rac) | cis-(rac) |
| I-114 | F | H | F | CH₂CF₃ | 5(rac) | trans-(rac) |
| I-115 | F | H | F | CH₂CF₃ | 5(rac) | 1R,3S (cis) |
| I-116 | F | H | F | CH₂CF₃ | 5(rac) | 1S,3R (cis) |
| I-117 | F | H | F | CH₂CF₃ | 5(rac) | 1R,3R (trans) |
| I-118 | F | H | F | CH₂CF₃ | 5(rac) | 1S,3S (trans) |
| I-119 | F | H | F | CH₂CF₃ | 5S | 1R,3S (cis) |
| I-120 | F | H | F | CH₂CF₃ | 5S | 1S,3R (cis) |
| I-121 | F | H | F | CH₂CF₃ | 5S | 1R,3R (trans) |
| I-122 | F | H | F | CH₂CF₃ | 5S | 1S,3S (trans) |
| I-123 | F | H | F | CH₂CF₃ | 5R | 1R,3S (cis) |
| I-124 | F | H | F | CH₂CF₃ | 5R | 1S,3R (cis) |
| I-125 | F | H | F | CH₂CF₃ | 5R | 1R,3R (trans) |
| I-126 | F | H | F | CH₂CF₃ | 5R | 1S,3S (trans) |
| I-127 | F | H | F | CH₂CH₂OMe | 5(rac) | cis-(rac) |
| I-128 | F | H | F | CH₂CH₂OMe | 5(rac) | trans-(rac) |
| I-129 | F | H | F | CH₂CH₂OMe | 5(rac) | 1R,3S (cis) |
| I-130 | F | H | F | CH₂CH₂OMe | 5(rac) | 1S,3R (cis) |
| I-131 | F | H | F | CH₂CH₂OMe | 5(rac) | 1R,3R (trans) |
| I-132 | F | H | F | CH₂CH₂OMe | 5(rac) | 1S,3S (trans) |
| I-133 | F | H | F | CH₂CH₂OMe | 5S | 1R,3S (cis) |
| I-134 | F | H | F | CH₂CH₂OMe | 5S | 1S,3R (cis) |
| I-135 | F | H | F | CH₂CH₂OMe | 5S | 1R,3R (trans) |
| I-136 | F | H | F | CH₂CH₂OMe | 5S | 1S,3S (trans) |
| I-137 | F | H | F | CH₂CH₂OMe | 5R | 1R,3S (cis) |
| I-138 | F | H | F | CH₂CH₂OMe | 5R | 1S,3R (cis) |
| I-139 | F | H | F | CH₂CH₂OMe | 5R | 1R,3R (trans) |
| I-140 | F | H | F | CH₂CH₂OMe | 5R | 1S,3S (trans) |
| I-141 | F | H | F | CH₂CH₂OEt | 5(rac) | cis-(rac) |
| I-142 | F | H | F | CH₂CH₂OEt | 5(rac) | trans-(rac) |
| I-143 | F | H | F | CH₂CH₂OEt | 5(rac) | 1R,3S (cis) |
| I-144 | F | H | F | CH₂CH₂OEt | 5(rac) | 1S,3R (cis) |
| I-145 | F | H | F | CH₂CH₂OEt | 5(rac) | 1R,3R (trans) |
| I-146 | F | H | F | CH₂CH₂OEt | 5(rac) | 1S,3S (trans) |
| I-147 | F | H | F | CH₂CH₂OEt | 5S | 1R,3S (cis) |
| I-148 | F | H | F | CH₂CH₂OEt | 5S | 1S,3R (cis) |
| I-149 | F | H | F | CH₂CH₂OEt | 5S | 1R,3R (trans) |
| I-150 | F | H | F | CH₂CH₂OEt | 5S | 1S,3S (trans) |
| I-151 | F | H | F | CH₂CH₂OEt | 5R | 1R,3S (cis) |
| I-152 | F | H | F | CH₂CH₂OEt | 5R | 1S,3R (cis) |
| I-153 | F | H | F | CH₂CH₂OEt | 5R | 1R,3R (trans) |
| I-154 | F | H | F | CH₂CH₂OEt | 5R | 1S,3S (trans) |
| I-155 | F | F | F | H | 5(rac) | cis-(rac) |
| I-156 | F | F | F | H | 5(rac) | trans-(rac) |
| I-157 | F | F | F | H | 5(rac) | 1R,3S (cis) |
| I-158 | F | F | F | H | 5(rac) | 1S,3R (cis) |
| I-159 | F | F | F | H | 5(rac) | 1R,3R (trans) |
| I-160 | F | F | F | H | 5(rac) | 1S,3S (trans) |
| I-161 | F | F | F | H | 5S | 1R,3S (cis) |
| I-162 | F | F | F | H | 5S | 1S,3R (cis) |
| I-163 | F | F | F | H | 5S | 1R,3R (trans) |
| I-164 | F | F | F | H | 5S | 1S,3S (trans) |
| I-165 | F | F | F | H | 5R | 1R,3S (cis) |
| I-166 | F | F | F | H | 5R | 1S,3R (cis) |
| I-167 | F | F | F | H | 5R | 1R,3R (trans) |
| I-168 | F | F | F | H | 5R | 1S,3S (trans) |
| I-169 | F | F | F | Me | 5(rac) | cis-(rac) |
| I-170 | F | F | F | Me | 5(rac) | trans-(rac) |
| I-171 | F | F | F | Me | 5(rac) | 1R,3S (cis) |
| I-172 | F | F | F | Me | 5(rac) | 1S,3R (cis) |
| I-173 | F | F | F | Me | 5(rac) | 1R,3R (trans) |
| I-174 | F | F | F | Me | 5(rac) | 1S,3S (trans) |
| I-175 | F | F | F | Me | 5S | 1R,3S (cis) |
| I-176 | F | F | F | Me | 5S | 1S,3R (cis) |
| I-177 | F | F | F | Me | 5S | 1R,3R (trans) |
| I-178 | F | F | F | Me | 5S | 1S,3S (trans) |
| I-179 | F | F | F | Me | 5R | 1R,3S (cis) |
| I-180 | F | F | F | Me | 5R | 1S,3R (cis) |
| I-181 | F | F | F | Me | 5R | 1R,3R (trans) |
| I-182 | F | F | F | Me | 5R | 1S,3S (trans) |
| I-183 | F | F | F | Et | 5(rac) | cis-(rac) |
| I-184 | F | F | F | Et | 5(rac) | trans-(rac) |
| I-185 | F | F | F | Et | 5(rac) | 1R,3S (cis) |
| I-186 | F | F | F | Et | 5(rac) | 1S,3R (cis) |
| I-187 | F | F | F | Et | 5(rac) | 1R,3R (trans) |
| I-188 | F | F | F | Et | 5(rac) | 1S,3S (trans) |
| I-189 | F | F | F | Et | 5S | 1R,3S (cis) |
| I-190 | F | F | F | Et | 5S | 1S,3R (cis) |
| I-191 | F | F | F | Et | 5S | 1R,3R (trans) |
| I-192 | F | F | F | Et | 5S | 1S,3S (trans) |
| I-193 | F | F | F | Et | 5R | 1R,3S (cis) |
| I-194 | F | F | F | Et | 5R | 1S,3R (cis) |
| I-195 | F | F | F | Et | 5R | 1R,3R (trans) |
| I-196 | F | F | F | Et | 5R | 1S,3S (trans) |
| I-197 | F | F | F | nPr | 5(rac) | cis-(rac) |
| I-198 | F | F | F | nPr | 5(rac) | trans-(rac) |
| I-199 | F | F | F | nPr | 5(rac) | 1R,3S (cis) |
| I-200 | F | F | F | nPr | 5(rac) | 1S,3R (cis) |
| I-201 | F | F | F | nPr | 5(rac) | 1R,3R (trans) |
| I-202 | F | F | F | nPr | 5(rac) | 1S,3S (trans) |
| I-203 | F | F | F | nPr | 5S | 1R,3S (cis) |
| I-204 | F | F | F | nPr | 5S | 1S,3R (cis) |
| I-205 | F | F | F | nPr | 5S | 1R,3R (trans) |
| I-206 | F | F | F | nPr | 5S | 1S,3S (trans) |
| I-207 | F | F | F | nPr | 5R | 1R,3S (cis) |
| I-208 | F | F | F | nPr | 5R | 1S,3R (cis) |
| I-209 | F | F | F | nPr | 5R | 1R,3R (trans) |
| I-210 | F | F | F | nPr | 5R | 1S,3S (trans) |
| I-211 | F | F | F | iPr | 5R/S | cis-R/S |
| I-212 | F | F | F | iPr | 5R/S | trans-R/S |
| I-213 | F | F | F | iPr | 5R/S | 1R,3S (cis) |
| I-214 | F | F | F | iPr | 5R/S | 1S,3R (cis) |
| I-215 | F | F | F | iPr | 5R/S | 1R,3R (trans) |
| I-216 | F | F | F | iPr | 5R/S | 1S,3S (trans) |
| I-217 | F | F | F | iPr | 5S | 1R,3S (cis) |
| I-218 | F | F | F | iPr | 5S | 1S,3R (cis) |
| I-219 | F | F | F | iPr | 5S | 1R,3R (trans) |
| I-220 | F | F | F | iPr | 5S | 1S,3S (trans) |
| I-221 | F | F | F | iPr | 5R | 1R,3S (cis) |
| I-222 | F | F | F | iPr | 5R | 1S,3R (cis) |
| I-223 | F | F | F | iPr | 5R | 1R,3R (trans) |
| I-224 | F | F | F | iPr | 5R | 1S,3S (trans) |
| I-225 | F | F | F | nBu | 5(rac) | cis-(rac) |
| I-226 | F | F | F | nBu | 5(rac) | trans-(rac) |
| I-227 | F | F | F | nBu | 5(rac) | 1R,3S (cis) |
| I-228 | F | F | F | nBu | 5(rac) | 1S,3R (cis) |
| I-229 | F | F | F | nBu | 5(rac) | 1R,3R (trans) |
| I-230 | F | F | F | nBu | 5(rac) | 1S,3S (trans) |
| I-231 | F | F | F | nBu | 5S | 1R,3S (cis) |
| I-232 | F | F | F | nBu | 5S | 1S,3R (cis) |
| I-233 | F | F | F | nBu | 5S | 1R,3R (trans) |
| I-234 | F | F | F | nBu | 5S | 1S,3S (trans) |
| I-235 | F | F | F | nBu | 5R | 1R,3S (cis) |
| I-236 | F | F | F | nBu | 5R | 1S,3R (cis) |
| I-237 | F | F | F | nBu | 5R | 1R,3R (trans) |
| I-238 | F | F | F | nBu | 5R | 1S,3S (trans) |
| I-239 | F | F | F | tBu | 5(rac) | cis-(rac) |
| I-240 | F | F | F | tBu | 5(rac) | trans-(rac) |
| I-241 | F | F | F | tBu | 5(rac) | 1R,3S (cis) |
| I-242 | F | F | F | tBu | 5(rac) | 1S,3R (cis) |
| I-243 | F | F | F | tBu | 5(rac) | 1R,3R (trans) |
| I-244 | F | F | F | tBu | 5(rac) | 1S,3S (trans) |
| I-245 | F | F | F | tBu | 5S | 1R,3S (cis) |
| I-246 | F | F | F | tBu | 5S | 1S,3R (cis) |
| I-247 | F | F | F | tBu | 5S | 1R,3R (trans) |
| I-248 | F | F | F | tBu | 5S | 1S,3S (trans) |
| I-249 | F | F | F | tBu | 5R | 1R,3S (cis) |
| I-250 | F | F | F | tBu | 5R | 1S,3R (cis) |
| I-251 | F | F | F | tBu | 5R | 1R,3R (trans) |
| I-252 | F | F | F | tBu | 5R | 1S,3S (trans) |
| I-253 | F | F | F | CH₂CH₂Cl | 5(rac) | cis-(rac) |
| I-254 | F | F | F | CH₂CH₂Cl | 5(rac) | trans-(rac) |
| I-255 | F | F | F | CH₂CH₂Cl | 5(rac) | 1R,3S (cis) |
| I-256 | F | F | F | CH₂CH₂Cl | 5(rac) | 1S,3R (cis) |
| I-257 | F | F | F | CH₂CH₂Cl | 5(rac) | 1R,3R (trans) |
| I-258 | F | F | F | CH₂CH₂Cl | 5(rac) | 1S,3S (trans) |
| I-259 | F | F | F | CH₂CH₂Cl | 5S | 1R,3S (cis) |
| I-260 | F | F | F | CH₂CH₂Cl | 5S | 1S,3R (cis) |
| I-261 | F | F | F | CH₂CH₂Cl | 5S | 1R,3R (trans) |
| I-262 | F | F | F | CH₂CH₂Cl | 5S | 1S,3S (trans) |
| I-263 | F | F | F | CH₂CH₂Cl | 5R | 1R,3S (cis) |
| I-264 | F | F | F | CH₂CH₂Cl | 5R | 1S,3R (cis) |
| I-265 | F | F | F | CH₂CH₂Cl | 5R | 1R,3R (trans) |
| I-266 | F | F | F | CH₂CH₂Cl | 5R | 1S,3S (trans) |
| I-267 | F | F | F | CH₂CF₃ | 5(rac) | cis-(rac) |
| I-268 | F | F | F | CH₂CF₃ | 5(rac) | trans-(rac) |
| I-269 | F | F | F | CH₂CF₃ | 5(rac) | 1R,3S (cis) |
| I-270 | F | F | F | CH₂CF₃ | 5(rac) | 1S,3R (cis) |
| I-271 | F | F | F | CH₂CF₃ | 5(rac) | 1R,3R (trans) |
| I-272 | F | F | F | CH₂CF₃ | 5(rac) | 1S,3S (trans) |
| I-273 | F | F | F | CH₂CF₃ | 5S | 1R,3S (cis) |
| I-274 | F | F | F | CH₂CF₃ | 5S | 1S,3R (cis) |
| I-275 | F | F | F | CH₂CF₃ | 5S | 1R,3R (trans) |
| I-276 | F | F | F | CH₂CF₃ | 5S | 1S,3S (trans) |
| I-277 | F | F | F | CH₂CF₃ | 5R | 1R,3S (cis) |
| I-278 | F | F | F | CH₂CF₃ | 5R | 1S,3R (cis) |
| I-279 | F | F | F | CH₂CF₃ | 5R | 1R,3R (trans) |
| I-280 | F | F | F | CH₂CF₃ | 5R | 1S,3S (trans) |
| I-281 | F | F | F | CH₂CH₂OMe | 5(rac) | cis-(rac) |
| I-282 | F | F | F | CH₂CH₂OMe | 5(rac) | trans-(rac) |
| I-283 | F | F | F | CH₂CH₂OMe | 5(rac) | 1R,3S (cis) |
| I-284 | F | F | F | CH₂CH₂OMe | 5(rac) | 1S,3R (cis) |
| I-285 | F | F | F | CH₂CH₂OMe | 5(rac) | 1R,3R (trans) |
| I-286 | F | F | F | CH₂CH₂OMe | 5(rac) | 1S,3S (trans) |
| I-287 | F | F | F | CH₂CH₂OMe | 5S | 1R,3S (cis) |
| I-288 | F | F | F | CH₂CH₂OMe | 5S | 1S,3R (cis) |
| I-289 | F | F | F | CH₂CH₂OMe | 5S | 1R,3R (trans) |
| I-290 | F | F | F | CH₂CH₂OMe | 5S | 1S,3S (trans) |
| I-291 | F | F | F | CH₂CH₂OMe | 5R | 1R,3S (cis) |
| I-292 | F | F | F | CH₂CH₂OMe | 5R | 1S,3R (cis) |
| I-293 | F | F | F | CH₂CH₂OMe | 5R | 1R,3R (trans) |
| I-294 | F | F | F | CH₂CH₂OMe | 5R | 1S,3S (trans) |
| I-295 | F | F | F | CH₂CH₂OEt | 5(rac) | cis-(rac) |
| I-296 | F | F | F | CH₂CH₂OEt | 5(rac) | trans-(rac) |
| I-297 | F | F | F | CH₂CH₂OEt | 5(rac) | 1R,3S (cis) |
| I-298 | F | F | F | CH₂CH₂OEt | 5(rac) | 1S,3R (cis) |
| I-299 | F | F | F | CH₂CH₂OEt | 5(rac) | 1R,3R (trans) |
| I-300 | F | F | F | CH₂CH₂OEt | 5(rac) | 1S,3S (trans) |
| I-301 | F | F | F | CH₂CH₂OEt | 5S | 1R,3S (cis) |
| I-302 | F | F | F | CH₂CH₂OEt | 5S | 1S,3R (cis) |
| I-303 | F | F | F | CH₂CH₂OEt | 5S | 1R,3R (trans) |
| I-304 | F | F | F | CH₂CH₂OEt | 5S | 1S,3S (trans) |
| I-305 | F | F | F | CH₂CH₂OEt | 5R | 1R,3S (cis) |
| I-306 | F | F | F | CH₂CH₂OEt | 5R | 1S,3R (cis) |
| I-307 | F | F | F | CH₂CH₂OEt | 5R | 1R,3R (trans) |
| I-308 | F | F | F | CH₂CH₂OEt | 5R | 1S,3S (trans) |
| I-309 | F | H | H | H | 5(rac) | cis-(rac) |
| I-310 | F | H | H | H | 5(rac) | trans-(rac) |
| I-311 | F | H | H | H | 5(rac) | 1R,3S (cis) |
| I-312 | F | H | H | H | 5(rac) | 1S,3R (cis) |
| I-313 | F | H | H | H | 5(rac) | 1R,3R (trans) |
| I-314 | F | H | H | H | 5(rac) | 1S,3S (trans) |
| I-315 | F | H | H | H | 5S | 1R,3S (cis) |
| I-316 | F | H | H | H | 5S | 1S,3R (cis) |
| I-317 | F | H | H | H | 5S | 1R,3R (trans) |
| I-318 | F | H | H | H | 5S | 1S,3S (trans) |
| I-319 | F | H | H | H | 5R | 1R,3S (cis) |
| I-320 | F | H | H | H | 5R | 1S,3R (cis) |
| I-321 | F | H | H | H | 5R | 1R,3R (trans) |
| I-322 | F | H | H | H | 5R | 1S,3S (trans) |
| I-323 | F | H | H | Me | 5(rac) | cis-(rac) |
| I-324 | F | H | H | Me | 5(rac) | trans-(rac) |
| I-325 | F | H | H | Me | 5(rac) | 1R,3S (cis) |
| I-326 | F | H | H | Me | 5(rac) | 1S,3R (cis) |
| I-327 | F | H | H | Me | 5(rac) | 1R,3R (trans) |
| I-328 | F | H | H | Me | 5(rac) | 1S,3S (trans) |
| I-329 | F | H | H | Me | 5S | 1R,3S (cis) |
| I-330 | F | H | H | Me | 5S | 1S,3R (cis) |
| I-331 | F | H | H | Me | 5S | 1R,3R (trans) |
| I-332 | F | H | H | Me | 5S | 1S,3S (trans) |
| I-333 | F | H | H | Me | 5R | 1R,3S (cis) |
| I-334 | F | H | H | Me | 5R | 1S,3R (cis) |
| I-335 | F | H | H | Me | 5R | 1R,3R (trans) |
| I-336 | F | H | H | Me | 5R | 1S,3S (trans) |
| I-337 | F | H | H | Et | 5(rac) | cis-(rac) |
| I-338 | F | H | H | Et | 5(rac) | trans-(rac) |
| I-339 | F | H | H | Et | 5(rac) | 1R,3S (cis) |
| I-340 | F | H | H | Et | 5(rac) | 1S,3R (cis) |
| I-341 | F | H | H | Et | 5(rac) | 1R,3R (trans) |
| I-342 | F | H | H | Et | 5(rac) | 1S,3S (trans) |
| I-343 | F | H | H | Et | 5S | 1R,3S (cis) |
| I-344 | F | H | H | Et | 5S | 1S,3R (cis) |
| I-345 | F | H | H | Et | 5S | 1R,3R (trans) |
| I-346 | F | H | H | Et | 5S | 1S,3S (trans) |
| I-347 | F | H | H | Et | 5R | 1R,3S (cis) |
| I-348 | F | H | H | Et | 5R | 1S,3R (cis) |
| I-349 | F | H | H | Et | 5R | 1R,3R (trans) |
| I-350 | F | H | H | Et | 5R | 1S,3S (trans) |
| I-351 | F | H | H | nPr | 5(rac) | cis-(rac) |
| I-352 | F | H | H | nPr | 5(rac) | trans-(rac) |
| I-353 | F | H | H | nPr | 5(rac) | 1R,3S (cis) |
| I-354 | F | H | H | nPr | 5(rac) | 1S,3R (cis) |
| I-355 | F | H | H | nPr | 5(rac) | 1R,3R (trans) |
| I-356 | F | H | H | nPr | 5(rac) | 1S,3S (trans) |
| I-357 | F | H | H | nPr | 5S | 1R,3S (cis) |
| I-358 | F | H | H | nPr | 5S | 1S,3R (cis) |
| I-359 | F | H | H | nPr | 5S | 1R,3R (trans) |
| I-360 | F | H | H | nPr | 5S | 1S,3S (trans) |
| I-361 | F | H | H | nPr | 5R | 1R,3S (cis) |
| I-362 | F | H | H | nPr | 5R | 1S,3R (cis) |
| I-363 | F | H | H | nPr | 5R | 1R,3R (trans) |
| I-364 | F | H | H | nPr | 5R | 1S,3S (trans) |
| I-365 | F | H | H | iPr | 5R/S | cis-R/S |
| I-366 | F | H | H | iPr | 5R/S | trans-R/S |
| I-367 | F | H | H | iPr | 5R/S | 1R,3S (cis) |
| I-368 | F | H | H | iPr | 5R/S | 1S,3R (cis) |
| I-369 | F | H | H | iPr | 5R/S | 1R,3R (trans) |
| I-370 | F | H | H | iPr | 5R/S | 1S,3S (trans) |
| I-371 | F | H | H | iPr | 5S | 1R,3S (cis) |
| I-372 | F | H | H | iPr | 5S | 1S,3R (cis) |
| I-373 | F | H | H | iPr | 5S | 1R,3R (trans) |
| I-374 | F | H | H | iPr | 5S | 1S,3S (trans) |
| I-375 | F | H | H | iPr | 5R | 1R,3S (cis) |
| I-376 | F | H | H | iPr | 5R | 1S,3R (cis) |
| I-377 | F | H | H | iPr | 5R | 1R,3R (trans) |
| I-378 | F | H | H | iPr | 5R | 1S,3S (trans) |
| I-379 | F | H | H | nBu | 5(rac) | cis-(rac) |
| I-380 | F | H | H | nBu | 5(rac) | trans-(rac) |
| I-381 | F | H | H | nBu | 5(rac) | 1R,3S (cis) |
| I-382 | F | H | H | nBu | 5(rac) | 1S,3R (cis) |
| I-383 | F | H | H | nBu | 5(rac) | 1R,3R (trans) |
| I-384 | F | H | H | nBu | 5(rac) | 1S,3S (trans) |
| I-385 | F | H | H | nBu | 5S | 1R,3S (cis) |
| I-386 | F | H | H | nBu | 5S | 1S,3R (cis) |
| I-387 | F | H | H | nBu | 5S | 1R,3R (trans) |
| I-388 | F | H | H | nBu | 5S | 1S,3S (trans) |
| I-389 | F | H | H | nBu | 5R | 1R,3S (cis) |
| I-390 | F | H | H | nBu | 5R | 1S,3R (cis) |
| I-391 | F | H | H | nBu | 5R | 1R,3R (trans) |
| I-392 | F | H | H | nBu | 5R | 1S,3S (trans) |
| I-393 | F | H | H | tBu | 5(rac) | cis-(rac) |
| I-394 | F | H | H | tBu | 5(rac) | trans-(rac) |
| I-395 | F | H | H | tBu | 5(rac) | 1R,3S (cis) |
| I-396 | F | H | H | tBu | 5(rac) | 1S,3R (cis) |
| I-397 | F | H | H | tBu | 5(rac) | 1R,3R (trans) |
| I-398 | F | H | H | tBu | 5(rac) | 1S,3S (trans) |
| I-399 | F | H | H | tBu | 5S | 1R,3S (cis) |
| I-400 | F | H | H | tBu | 5S | 1S,3R (cis) |
| I-401 | F | H | H | tBu | 5S | 1R,3R (trans) |
| I-402 | F | H | H | tBu | 5S | 1S,3S (trans) |
| I-403 | F | H | H | tBu | 5R | 1R,3S (cis) |
| I-404 | F | H | H | tBu | 5R | 1S,3R (cis) |
| I-405 | F | H | H | tBu | 5R | 1R,3R (trans) |
| I-406 | F | H | H | tBu | 5R | 1S,3S (trans) |
| I-407 | F | H | H | CH₂CH₂Cl | 5(rac) | cis-(rac) |
| I-408 | F | H | H | CH₂CH₂Cl | 5(rac) | trans-(rac) |
| I-409 | F | H | H | CH₂CH₂Cl | 5(rac) | 1R,3S (cis) |
| I-410 | F | H | H | CH₂CH₂Cl | 5(rac) | 1S,3R (cis) |
| I-411 | F | H | H | CH₂CH₂Cl | 5(rac) | 1R,3R (trans) |
| I-412 | F | H | H | CH₂CH₂Cl | 5(rac) | 1S,3S (trans) |
| I-413 | F | H | H | CH₂CH₂Cl | 5S | 1R,3S (cis) |
| I-414 | F | H | H | CH₂CH₂Cl | 5S | 1S,3R (cis) |
| I-415 | F | H | H | CH₂CH₂Cl | 5S | 1R,3R (trans) |
| I-416 | F | H | H | CH₂CH₂Cl | 5S | 1S,3S (trans) |
| I-417 | F | H | H | CH₂CH₂Cl | 5R | 1R,3S (cis) |
| I-418 | F | H | H | CH₂CH₂Cl | 5R | 1S,3R (cis) |
| I-419 | F | H | H | CH₂CH₂Cl | 5R | 1R,3R (trans) |
| I-420 | F | H | H | CH₂CH₂Cl | 5R | 1S,3S (trans) |
| I-421 | F | H | H | CH₂CF₃ | 5(rac) | cis-(rac) |
| I-422 | F | H | H | CH₂CF₃ | 5(rac) | trans-(rac) |
| I-423 | F | H | H | CH₂CF₃ | 5(rac) | 1R,3S (cis) |
| I-424 | F | H | H | CH₂CF₃ | 5(rac) | 1S,3R (cis) |
| I-425 | F | H | H | CH₂CF₃ | 5(rac) | 1R,3R (trans) |
| I-426 | F | H | H | CH₂CF₃ | 5(rac) | 1S,3S (trans) |
| I-427 | F | H | H | CH₂CF₃ | 5S | 1R,3S (cis) |
| I-428 | F | H | H | CH₂CF₃ | 5S | 1S,3R (cis) |
| I-429 | F | H | H | CH₂CF₃ | 5S | 1R,3R (trans) |
| I-430 | F | H | H | CH₂CF₃ | 5S | 1S,3S (trans) |
| I-431 | F | H | H | CH₂CF₃ | 5R | 1R,3S (cis) |
| I-432 | F | H | H | CH₂CF₃ | 5R | 1S,3R (cis) |
| I-433 | F | H | H | CH₂CF₃ | 5R | 1R,3R (trans) |
| I-434 | F | H | H | CH₂CF₃ | 5R | 1S,3S (trans) |
| I-435 | F | H | H | CH₂CH₂OMe | 5(rac) | cis-(rac) |
| I-436 | F | H | H | CH₂CH₂OMe | 5(rac) | trans-(rac) |
| I-437 | F | H | H | CH₂CH₂OMe | 5(rac) | 1R,3S (cis) |
| I-438 | F | H | H | CH₂CH₂OMe | 5(rac) | 1S,3R (cis) |
| I-439 | F | H | H | CH₂CH₂OMe | 5(rac) | 1R,3R (trans) |
| I-440 | F | H | H | CH₂CH₂OMe | 5(rac) | 1S,3S (trans) |
| I-441 | F | H | H | CH₂CH₂OMe | 5S | 1R,3S (cis) |
| I-442 | F | H | H | CH₂CH₂OMe | 5S | 1S,3R (cis) |
| I-443 | F | H | H | CH₂CH₂OMe | 5S | 1R,3R (trans) |
| I-444 | F | H | H | CH₂CH₂OMe | 5S | 1S,3S (trans) |
| I-445 | F | H | H | CH₂CH₂OMe | 5R | 1R,3S (cis) |
| I-446 | F | H | H | CH₂CH₂OMe | 5R | 1S,3R (cis) |
| I-447 | F | H | H | CH₂CH₂OMe | 5R | 1R,3R (trans) |
| I-448 | F | H | H | CH₂CH₂OMe | 5R | 1S,3S (trans) |
| I-449 | F | H | H | CH₂CH₂OEt | 5(rac) | cis-(rac) |
| I-450 | F | H | H | CH₂CH₂OEt | 5(rac) | trans-(rac) |
| I-451 | F | H | H | CH₂CH₂OEt | 5(rac) | 1R,3S (cis) |
| I-452 | F | H | H | CH₂CH₂OEt | 5(rac) | 1S,3R (cis) |
| I-453 | F | H | H | CH₂CH₂OEt | 5(rac) | 1R,3R (trans) |
| I-454 | F | H | H | CH₂CH₂OEt | 5(rac) | 1S,3S (trans) |
| I-455 | F | H | H | CH₂CH₂OEt | 5S | 1R,3S (cis) |
| I-456 | F | H | H | CH₂CH₂OEt | 5S | 1S,3R (cis) |
| I-457 | F | H | H | CH₂CH₂OEt | 5S | 1R,3R (trans) |
| I-458 | F | H | H | CH₂CH₂OEt | 5S | 1S,3S (trans) |
| I-459 | F | H | H | CH₂CH₂OEt | 5R | 1R,3S(cis) |
| I-460 | F | H | H | CH₂CH₂OEt | 5R | 1S,3R (cis) |
| I-461 | F | H | H | CH₂CH₂OEt | 5R | 1R,3R (trans) |
| I-462 | F | H | H | CH₂CH₂OEt | 5R | 1S,3S (trans) |
| I-463 | F | F | H | H | 5(rac) | cis-(rac) |
| I-464 | F | F | H | H | 5(rac) | trans-(rac) |
| I-465 | F | F | H | H | 5(rac) | 1R,3S (cis) |
| I-466 | F | F | H | H | 5(rac) | 1S,3S (trans) |
| I-467 | F | F | H | H | 5R | 1R,3S (cis) |
| I-468 | F | F | H | H | 5R | 1S,3S (trans) |
| I-469 | F | F | H | Me | 5(rac) | cis-(rac) |
| I-470 | F | F | H | Me | 5(rac) | trans-(rac) |
| I-471 | F | F | H | Me | 5(rac) | 1R,3S (cis) |
| I-472 | F | F | H | Me | 5(rac) | 1S,3S (trans) |
| I-473 | F | F | H | Me | 5R | 1R,3S (cis) |
| I-474 | F | F | H | Me | 5R | 1S,3S (trans) |
| I-475 | F | F | H | Et | 5(rac) | cis-(rac) |
| I-476 | F | F | H | Et | 5(rac) | trans-(rac) |
| I-477 | F | F | H | Et | 5(rac) | 1R,3S (cis) |
| I-478 | F | F | H | Et | 5(rac) | 1S,3S (trans) |
| I-479 | F | F | H | Et | 5R | 1R,3S (cis) |
| I-480 | F | F | H | Et | 5R | 1S,3S (trans) |
| I-481 | F | F | H | nPr | 5(rac) | cis-(rac) |
| I-482 | F | F | H | nPr | 5(rac) | trans-(rac) |
| I-483 | F | F | H | nPr | 5(rac) | 1R,3S (cis) |
| I-484 | F | F | H | nPr | 5(rac) | 1S,3S (trans) |
| I-485 | F | F | H | nPr | 5R | 1R,3S (cis) |
| I-486 | F | F | H | nPr | 5R | 1S,3S (trans) |
| I-487 | F | F | H | iPr | 5R/S | cis-R/S |
| I-488 | F | F | H | iPr | 5(rac) | cis-(rac) |
| I-489 | F | F | H | iPr | 5(rac) | trans-(rac) |
| I-490 | F | F | H | iPr | 5(rac) | 1R,3S (cis) |
| I-491 | F | F | H | iPr | 5(rac) | 1S,3S (trans) |
| I-492 | F | F | H | iPr | 5R | 1R,3S (cis) |
| I-493 | F | F | H | nBu | 5(rac) | cis-(rac) |
| I-494 | F | F | H | nBu | 5(rac) | trans-(rac) |
| I-495 | F | F | H | nBu | 5(rac) | 1R,3S (cis) |
| I-496 | F | F | H | nBu | 5(rac) | 1S,3S (trans) |
| I-497 | F | F | H | nBu | 5R | 1R,3S (cis) |
| I-498 | F | F | H | nBu | 5R | 1S,3S (trans) |
| I-499 | F | F | H | tBu | 5(rac) | cis-(rac) |
| I-500 | F | F | H | tBu | 5(rac) | trans-(rac) |
| I-501 | F | F | H | tBu | 5(rac) | 1R,3S (cis) |
| I-502 | F | F | H | tBu | 5(rac) | 1S,3S (trans) |
| I-503 | F | F | H | tBu | 5R | 1R,3S (cis) |
| I-504 | F | F | H | tBu | 5R | 1S,3S (trans) |
| I-505 | F | F | H | CH₂CH₂Cl | 5(rac) | cis-(rac) |
| I-506 | F | F | H | CH₂CH₂Cl | 5(rac) | trans-(rac) |
| I-507 | F | F | H | CH₂CH₂Cl | 5(rac) | 1R,3S (cis) |
| I-508 | F | F | H | CH₂CH₂Cl | 5(rac) | 1S,3S (trans) |
| I-509 | F | F | H | CH₂CH₂Cl | 5R | 1R,3S (cis) |
| I-510 | F | F | H | CH₂CH₂Cl | 5R | 1S,3S (trans) |
| I-511 | F | F | H | CH₂CF₃ | 5(rac) | cis-(rac) |
| I-512 | F | F | H | CH₂CF₃ | 5(rac) | trans-(rac) |
| I-513 | F | F | H | CH₂CF₃ | 5(rac) | 1R,3S (cis) |
| I-514 | F | F | H | CH₂CF₃ | 5(rac) | 1S,3S (trans) |
| I-515 | F | F | H | CH₂CF₃ | 5R | 1R,3S (cis) |
| I-516 | F | F | H | CH₂CF₃ | 5R | 1S,3S (trans) |
| I-517 | F | F | H | CH₂CH₂OMe | 5(rac) | cis-(rac) |
| I-518 | F | F | H | CH₂CH₂OMe | 5(rac) | trans-(rac) |
| I-519 | F | F | H | CH₂CH₂OMe | 5(rac) | 1R,3S (cis) |
| I-520 | F | F | H | CH₂CH₂OMe | 5(rac) | 1S,3S (trans) |
| I-521 | F | F | H | CH₂CH₂OMe | 5R | 1R,3S (cis) |
| I-522 | F | F | H | CH₂CH₂OMe | 5R | 1S,3S (trans) |
| I-523 | F | F | H | CH₂CH₂OEt | 5(rac) | cis-(rac) |
| I-524 | F | F | H | CH₂CH₂OEt | 5(rac) | trans-(rac) |
| I-525 | F | F | H | CH₂CH₂OEt | 5(rac) | 1R,3S (cis) |
| I-526 | F | F | H | CH₂CH₂OEt | 5(rac) | 1S,3S (trans) |
| I-527 | F | F | H | CH₂CH₂OEt | 5R | 1R,3S (cis) |
| I-528 | F | F | H | CH₂CH₂OEt | 5R | 1S,3S (trans) |
| I-529 | F | H | Me | H | 5(rac) | cis-(rac) |
| I-530 | F | H | Me | H | 5(rac) | trans-(rac) |
| I-531 | F | H | Me | H | 5(rac) | 1R,3S (cis) |
| I-532 | F | H | Me | H | 5(rac) | 1S,3S (trans) |
| I-533 | F | H | Me | H | 5R | 1R,3S (cis) |
| I-534 | F | H | Me | H | 5R | 1S,3S (trans) |
| I-535 | F | H | Me | Me | 5(rac) | cis-(rac) |
| I-536 | F | H | Me | Me | 5(rac) | trans-(rac) |
| I-537 | F | H | Me | Me | 5(rac) | 1R,3S (cis) |
| I-538 | F | H | Me | Me | 5(rac) | 1S,3S (trans) |
| I-539 | F | H | Me | Me | 5R | 1R,3S (cis) |
| I-540 | F | H | Me | Me | 5R | 1S,3S (trans) |
| I-541 | F | H | Me | Et | 5(rac) | cis-(rac) |
| I-542 | F | H | Me | Et | 5(rac) | trans-(rac) |
| I-543 | F | H | Me | Et | 5(rac) | 1R,3S (cis) |
| I-544 | F | H | Me | Et | 5(rac) | 1S,3S (trans) |
| I-545 | F | H | Me | Et | 5R | 1R,3S (cis) |
| I-546 | F | H | Me | Et | 5R | 1S,3S (trans) |
| I-547 | F | H | Me | nPr | 5(rac) | cis-(rac) |
| I-548 | F | H | Me | nPr | 5(rac) | trans-(rac) |
| I-549 | F | H | Me | nPr | 5(rac) | 1R,3S (cis) |
| I-550 | F | H | Me | nPr | 5(rac) | 1S,3S (trans) |
| I-551 | F | H | Me | nPr | 5R | 1R,3S (cis) |
| I-552 | F | H | Me | nPr | 5R | 1S,3S (trans) |
| I-553 | F | H | Me | iPr | 5R/S | cis-R/S |
| I-554 | F | H | Me | iPr | 5(rac) | cis-(rac) |
| I-555 | F | H | Me | iPr | 5(rac) | trans-(rac) |
| I-556 | F | H | Me | iPr | 5(rac) | 1R,3S (cis) |
| I-557 | F | H | Me | iPr | 5(rac) | 1S,3S (trans) |
| I-558 | F | H | Me | iPr | 5R | 1R,3S (cis) |
| I-560 | F | H | Me | nBu | 5(rac) | cis-(rac) |
| I-561 | F | H | Me | nBu | 5(rac) | trans-(rac) |
| I-562 | F | H | Me | nBu | 5(rac) | 1R,3S (cis) |
| I-563 | F | H | Me | nBu | 5(rac) | 1S,3S (trans) |
| I-564 | F | H | Me | nBu | 5R | 1R,3S (cis) |
| I-565 | F | H | Me | nBu | 5R | 1S,3S (trans) |
| I-566 | F | H | Me | tBu | 5(rac) | cis-(rac) |
| I-567 | F | H | Me | tBu | 5(rac) | trans-(rac) |
| I-568 | F | H | Me | tBu | 5(rac) | 1R,3S (cis) |
| I-569 | F | H | Me | tBu | 5(rac) | 1S,3S (trans) |
| I-570 | F | H | Me | tBu | 5R | 1R,3S (cis) |
| I-571 | F | H | Me | tBu | 5R | 1S,3S (trans) |
| I-572 | F | H | Me | CH₂CH₂Cl | 5(rac) | cis-(rac) |
| I-573 | F | H | Me | CH₂CH₂Cl | 5(rac) | trans-(rac) |
| I-574 | F | H | Me | CH₂CH₂Cl | 5(rac) | 1R,3S (cis) |
| I-575 | F | H | Me | CH₂CH₂Cl | 5(rac) | 1S,3S (trans) |
| I-576 | F | H | Me | CH₂CH₂Cl | 5R | 1R,3S (cis) |
| I-577 | F | H | Me | CH₂CH₂Cl | 5R | 1S,3S (trans) |
| I-578 | F | H | Me | CH₂CF₃ | 5(rac) | cis-(rac) |
| I-579 | F | H | Me | CH₂CF₃ | 5(rac) | trans-(rac) |
| I-580 | F | H | Me | CH₂CF₃ | 5(rac) | 1R,3S (cis) |
| I-581 | F | H | Me | CH₂CF₃ | 5(rac) | 1S,3S (trans) |
| I-582 | F | H | Me | CH₂CF₃ | 5R | 1R,3S (cis) |
| I-583 | F | H | Me | CH₂CF₃ | 5R | 1S,3S (trans) |
| I-584 | F | H | Me | CH₂CH₂OMe | 5(rac) | cis-(rac) |
| I-585 | F | H | Me | CH₂CH₂OMe | 5(rac) | trans-(rac) |
| I-586 | F | H | Me | CH₂CH₂OMe | 5(rac) | 1R,3S (cis) |
| I-587 | F | H | Me | CH₂CH₂OMe | 5(rac) | 1S,3S (trans) |
| I-588 | F | H | Me | CH₂CH₂OMe | 5R | 1R,3S (cis) |
| I-589 | F | H | Me | CH₂CH₂OMe | 5R | 1S,3S (trans) |
| I-590 | F | H | Me | CH₂CH₂OEt | 5(rac) | cis-(rac) |
| I-591 | F | H | Me | CH₂CH₂OEt | 5(rac) | trans-(rac) |
| I-592 | F | H | Me | CH₂CH₂OEt | 5(rac) | 1R,3S (cis) |
| I-593 | F | H | Me | CH₂CH₂OEt | 5(rac) | 1S,3S (trans) |
| I-594 | F | H | Me | CH₂CH₂OEt | 5R | 1R,3S (cis) |
| I-595 | F | H | Me | CH₂CH₂OEt | 5R | 1S,3S (trans) |
| I-596 | F | H | CN | H | 5(rac) | cis-(rac) |
| I-597 | F | H | CN | H | 5(rac) | trans-(rac) |
| I-598 | F | H | CN | H | 5(rac) | 1R,3S (cis) |
| I-599 | F | H | CN | H | 5(rac) | 1S,3S (trans) |
| I-600 | F | H | CN | H | 5R | 1R,3S (cis) |
| I-601 | F | H | CN | H | 5R | 1S,3S (trans) |
| I-602 | F | H | CN | Me | 5(rac) | cis-(rac) |
| I-603 | F | H | CN | Me | 5(rac) | trans-(rac) |
| I-604 | F | H | CN | Me | 5(rac) | 1R,3S (cis) |
| I-605 | F | H | CN | Me | 5(rac) | 1S,3S (trans) |
| I-606 | F | H | CN | Me | 5R | 1R,3S (cis) |
| I-607 | F | H | CN | Me | 5R | 1S,3S (trans) |
| I-608 | F | H | CN | Et | 5(rac) | cis-(rac) |
| I-609 | F | H | CN | Et | 5(rac) | trans-(rac) |
| I-610 | F | H | CN | Et | 5(rac) | 1R,3S (cis) |
| I-611 | F | H | CN | Et | 5(rac) | 1S,3S (trans) |
| I-612 | F | H | CN | Et | 5R | 1R,3S (cis) |
| I-613 | F | H | CN | Et | 5R | 1S,3S (trans) |
| I-614 | F | H | CN | nPr | 5(rac) | cis-(rac) |
| I-615 | F | H | CN | nPr | 5(rac) | trans-(rac) |
| I-616 | F | H | CN | nPr | 5(rac) | 1R,3S (cis) |
| I-617 | F | H | CN | nPr | 5(rac) | 1S,3S (trans) |
| I-618 | F | H | CN | nPr | 5R | 1R,3S (cis) |
| I-619 | F | H | CN | nPr | 5R | 1S,3S (trans) |
| I-620 | F | H | CN | iPr | 5R/S | cis-R/S |
| I-621 | F | H | CN | iPr | 5(rac) | cis-(rac) |
| I-622 | F | H | CN | iPr | 5(rac) | trans-(rac) |
| I-623 | F | H | CN | iPr | 5(rac) | 1R,3S (cis) |
| I-624 | F | H | CN | iPr | 5(rac) | 1S,3S (trans) |
| I-625 | F | H | CN | iPr | 5R | 1R,3S (cis) |
| I-626 | F | H | CN | nBu | 5(rac) | cis-(rac) |
| I-627 | F | H | CN | nBu | 5(rac) | trans-(rac) |
| I-628 | F | H | CN | nBu | 5(rac) | 1R,3S (cis) |
| I-629 | F | H | CN | nBu | 5(rac) | 1S,3S (trans) |
| I-630 | F | H | CN | nBu | 5R | 1R,3S (cis) |
| I-631 | F | H | CN | nBu | 5R | 1S,3S (trans) |
| I-632 | F | H | CN | tBu | 5(rac) | cis-(rac) |
| I-633 | F | H | CN | tBu | 5(rac) | trans-(rac) |
| I-634 | F | H | CN | tBu | 5(rac) | 1R,3S (cis) |
| I-635 | F | H | CN | tBu | 5(rac) | 1S,3S (trans) |
| I-636 | F | H | CN | tBu | 5R | 1R,3S (cis) |
| I-637 | F | H | CN | tBu | 5R | 1S,3S (trans) |
| I-638 | F | H | CN | CH₂CH₂Cl | 5(rac) | cis-(rac) |
| I-639 | F | H | CN | CH₂CH₂Cl | 5(rac) | trans-(rac) |
| I-640 | F | H | CN | CH₂CH₂Cl | 5(rac) | 1R,3S (cis) |
| I-641 | F | H | CN | CH₂CH₂Cl | 5(rac) | 1S,3S (trans) |
| I-642 | F | H | CN | CH₂CH₂Cl | 5R | 1R,3S (cis) |
| I-643 | F | H | CN | CH₂CH₂Cl | 5R | 1S,3S (trans) |
| I-644 | F | H | CN | CH₂CF₃ | 5(rac) | cis-(rac) |
| I-645 | F | H | CN | CH₂CF₃ | 5(rac) | trans-(rac) |
| I-646 | F | H | CN | CH₂CF₃ | 5(rac) | 1R,3S (cis) |
| I-647 | F | H | CN | CH₂CF₃ | 5(rac) | 1S,3S (trans) |
| I-648 | F | H | CN | CH₂CF₃ | 5R | 1R,3S (cis) |
| I-649 | F | H | CN | CH₂CF₃ | 5R | 1S,3S (trans) |
| I-650 | F | H | CN | CH₂CH₂OMe | 5(rac) | cis-(rac) |
| I-651 | F | H | CN | CH₂CH₂OMe | 5(rac) | trans-(rac) |
| I-652 | F | H | CN | CH₂CH₂OMe | 5(rac) | 1R,3S (cis) |
| I-653 | F | H | CN | CH₂CH₂OMe | 5(rac) | 1S,3S (trans) |
| I-654 | F | H | CN | CH₂CH₂OMe | 5R | 1R,3S (cis) |
| I-655 | F | H | CN | CH₂CH₂OMe | 5R | 1S,3S (trans) |
| I-656 | F | H | CN | CH₂CH₂OEt | 5(rac) | cis-(rac) |
| I-657 | F | H | CN | CH₂CH₂OEt | 5(rac) | trans-(rac) |
| I-658 | F | H | CN | CH₂CH₂OEt | 5(rac) | 1R,3S (cis) |
| I-659 | F | H | CN | CH₂CH₂OEt | 5(rac) | 1S,3S (trans) |
| I-660 | F | H | CN | CH₂CH₂OEt | 5R | 1R,3S (cis) |
| I-661 | F | H | CN | CH₂CH₂OEt | 5R | 1S,3S (trans) |
| I-662 | Cl | H | Cl | H | 5(rac) | cis-(rac) |
| I-663 | Cl | H | Cl | H | 5(rac) | trans-(rac) |
| I-664 | Cl | H | Cl | H | 5(rac) | 1R,3S (cis) |
| I-665 | Cl | H | Cl | H | 5(rac) | 1S,3S (trans) |
| I-666 | Cl | H | Cl | H | 5R | 1R,3S (cis) |
| I-667 | Cl | H | Cl | H | 5R | 1S,3S (trans) |
| I-668 | Cl | H | Cl | Me | 5(rac) | cis-(rac) |
| I-669 | Cl | H | Cl | Me | 5(rac) | trans-(rac) |
| I-670 | Cl | H | Cl | Me | 5(rac) | 1R,3S (cis) |
| I-671 | Cl | H | Cl | Me | 5(rac) | 1S,3S (trans) |
| I-672 | Cl | H | Cl | Me | 5R | 1R,3S (cis) |
| I-673 | Cl | H | Cl | Me | 5R | 1S,3S (trans) |
| I-674 | Cl | H | Cl | Et | 5(rac) | cis-(rac) |
| I-675 | Cl | H | Cl | Et | 5(rac) | trans-(rac) |
| I-676 | Cl | H | Cl | Et | 5(rac) | 1R,3S (cis) |
| I-677 | Cl | H | Cl | Et | 5(rac) | 1S,3S (trans) |
| I-678 | Cl | H | Cl | Et | 5R | 1R,3S (cis) |
| I-679 | Cl | H | Cl | Et | 5R | 1S,3S (trans) |
| I-680 | Cl | H | Cl | nPr | 5(rac) | cis-(rac) |
| I-681 | Cl | H | Cl | nPr | 5(rac) | trans-(rac) |
| I-682 | Cl | H | Cl | nPr | 5(rac) | 1R,3S (cis) |
| I-683 | Cl | H | Cl | nPr | 5(rac) | 1S,3S (trans) |
| I-684 | Cl | H | Cl | nPr | 5R | 1R,3S (cis) |
| I-685 | Cl | H | Cl | nPr | 5R | 1S,3S (trans) |
| I-686 | Cl | H | Cl | iPr | 5R/S | cis-R/S |
| I-687 | Cl | H | Cl | iPr | 5(rac) | cis-(rac) |
| I-688 | Cl | H | Cl | iPr | 5(rac) | trans-(rac) |
| I-689 | Cl | H | Cl | iPr | 5(rac) | 1R,3S (cis) |
| I-690 | Cl | H | Cl | iPr | 5(rac) | 1S,3S (trans) |
| I-691 | Cl | H | Cl | iPr | 5R | 1R,3S (cis) |
| I-692 | Cl | H | Cl | nBu | 5(rac) | cis-(rac) |
| I-693 | Cl | H | Cl | nBu | 5(rac) | trans-(rac) |
| I-694 | Cl | H | Cl | nBu | 5(rac) | 1R,3S (cis) |
| I-695 | Cl | H | Cl | nBu | 5(rac) | 1S,3S (trans) |
| I-696 | Cl | H | Cl | nBu | 5R | 1R,3S (cis) |
| I-697 | Cl | H | Cl | nBu | 5R | 1S,3S (trans) |
| I-698 | Cl | H | Cl | tBu | 5(rac) | cis-(rac) |
| I-699 | Cl | H | Cl | tBu | 5(rac) | trans-(rac) |
| I-700 | Cl | H | Cl | tBu | 5(rac) | 1R,3S (cis) |
| I-701 | Cl | H | Cl | tBu | 5(rac) | 1S,3S (trans) |
| I-702 | Cl | H | Cl | tBu | 5R | 1R,3S (cis) |
| I-703 | Cl | H | Cl | tBu | 5R | 1S,3S (trans) |
| I-704 | Cl | H | Cl | CH₂CH₂Cl | 5(rac) | cis-(rac) |
| I-705 | Cl | H | Cl | CH₂CH₂Cl | 5(rac) | trans-(rac) |
| I-706 | Cl | H | Cl | CH₂CH₂Cl | 5(rac) | 1R,3S (cis) |
| I-707 | Cl | H | Cl | CH₂CH₂Cl | 5(rac) | 1S,3S (trans) |
| I-708 | Cl | H | Cl | CH₂CH₂Cl | 5R | 1R,3S (cis) |
| I-709 | Cl | H | Cl | CH₂CH₂Cl | 5R | 1S,3S (trans) |
| I-710 | Cl | H | Cl | CH₂CF₃ | 5(rac) | cis-(rac) |
| I-711 | Cl | H | Cl | CH₂CF₃ | 5(rac) | trans-(rac) |
| I-712 | Cl | H | Cl | CH₂CF₃ | 5(rac) | 1R,3S (cis) |
| I-713 | Cl | H | Cl | CH₂CF₃ | 5(rac) | 1S,3S (trans) |
| I-714 | Cl | H | Cl | CH₂CF₃ | 5R | 1R,3S (cis) |
| I-715 | Cl | H | Cl | CH₂CF₃ | 5R | 1S,3S (trans) |
| I-716 | Cl | H | Cl | CH₂CH₂OMe | 5(rac) | cis-(rac) |
| I-717 | Cl | H | Cl | CH₂CH₂OMe | 5(rac) | trans-(rac) |
| I-718 | Cl | H | Cl | CH₂CH₂OMe | 5(rac) | 1R,3S (cis) |
| I-719 | Cl | H | Cl | CH₂CH₂OMe | 5(rac) | 1S,3S (trans) |
| I-720 | Cl | H | Cl | CH₂CH₂OMe | 5R | 1R,3S (cis) |
| I-721 | Cl | H | Cl | CH₂CH₂OMe | 5R | 1S,3S (trans) |
| I-722 | Cl | H | Cl | CH₂CH₂OEt | 5(rac) | cis-(rac) |
| I-723 | Cl | H | Cl | CH₂CH₂OEt | 5(rac) | trans-(rac) |
| I-724 | Cl | H | Cl | CH₂CH₂OEt | 5(rac) | 1R,3S (cis) |
| I-725 | Cl | H | Cl | CH₂CH₂OEt | 5(rac) | 1S,3S (trans) |
| I-726 | Cl | H | Cl | CH₂CH₂OEt | 5R | 1R,3S (cis) |
| I-727 | Cl | H | Cl | CH₂CH₂OEt | 5R | 1S,3S (trans) |
| I-728 | Cl | H | H | H | 5(rac) | cis-(rac) |
| I-729 | Cl | H | H | H | 5(rac) | trans-(rac) |
| I-730 | Cl | H | H | H | 5(rac) | 1R,3S (cis) |
| I-731 | Cl | H | H | H | 5(rac) | 1S,3S (trans) |
| I-732 | Cl | H | H | H | 5R | 1R,3S (cis) |
| I-733 | Cl | H | H | H | 5R | 1S,3S (trans) |
| I-734 | Cl | H | H | Me | 5(rac) | cis-(rac) |
| I-735 | Cl | H | H | Me | 5(rac) | trans-(rac) |
| I-736 | Cl | H | H | Me | 5(rac) | 1R,3S (cis) |
| I-737 | Cl | H | H | Me | 5(rac) | 1S,3S (trans) |
| I-738 | Cl | H | H | Me | 5R | 1R,3S (cis) |
| I-739 | Cl | H | H | Me | 5R | 1S,3S (trans) |
| I-740 | Cl | H | H | Et | 5(rac) | cis-(rac) |
| I-741 | Cl | H | H | Et | 5(rac) | trans-(rac) |
| I-742 | Cl | H | H | Et | 5(rac) | 1R,3S (cis) |
| I-743 | Cl | H | H | Et | 5(rac) | 1S,3S (trans) |
| I-744 | Cl | H | H | Et | 5R | 1R,3S (cis) |
| I-745 | Cl | H | H | Et | 5R | 1S,3S (trans) |
| I-746 | Cl | H | H | nPr | 5(rac) | cis-(rac) |
| I-747 | Cl | H | H | nPr | 5(rac) | trans-(rac) |
| I-748 | Cl | H | H | nPr | 5(rac) | 1R,3S (cis) |
| I-749 | Cl | H | H | nPr | 5(rac) | 1S,3S (trans) |
| I-750 | Cl | H | H | nPr | 5R | 1R,3S (cis) |
| I-751 | Cl | H | H | nPr | 5R | 1S,3S (trans) |
| I-752 | Cl | H | H | iPr | 5R/S | cis-R/S |
| I-753 | Cl | H | H | iPr | 5(rac) | cis-(rac) |
| I-754 | Cl | H | H | iPr | 5(rac) | trans-(rac) |
| I-755 | Cl | H | H | iPr | 5(rac) | 1R,3S (cis) |
| I-756 | Cl | H | H | iPr | 5(rac) | 1S,3S (trans) |
| I-757 | Cl | H | H | iPr | 5R | 1R,3S (cis) |
| I-758 | Cl | H | H | nBu | 5(rac) | cis-(rac) |
| I-759 | Cl | H | H | nBu | 5(rac) | trans-(rac) |
| I-760 | Cl | H | H | nBu | 5(rac) | 1R,3S (cis) |
| I-761 | Cl | H | H | nBu | 5(rac) | 1S,3S (trans) |
| I-762 | Cl | H | H | nBu | 5R | 1R,3S (cis) |
| I-763 | Cl | H | H | nBu | 5R | 1S,3S (trans) |
| I-764 | Cl | H | H | tBu | 5(rac) | cis-(rac) |
| I-765 | Cl | H | H | tBu | 5(rac) | trans-(rac) |
| I-766 | Cl | H | H | tBu | 5(rac) | 1R,3S (cis) |
| I-767 | Cl | H | H | tBu | 5(rac) | 1S,3S (trans) |
| I-768 | Cl | H | H | tBu | 5R | 1R,3S (cis) |
| I-769 | Cl | H | H | tBu | 5R | 1S,3S (trans) |
| I-770 | Cl | H | H | CH₂CH₂Cl | 5(rac) | cis-(rac) |
| I-771 | Cl | H | H | CH₂CH₂Cl | 5(rac) | trans-(rac) |
| I-772 | Cl | H | H | CH₂CH₂Cl | 5(rac) | 1R,3S (cis) |
| I-773 | Cl | H | H | CH₂CH₂Cl | 5(rac) | 1S,3S (trans) |
| I-774 | Cl | H | H | CH₂CH₂Cl | 5R | 1R,3S (cis) |
| I-775 | Cl | H | H | CH₂CH₂Cl | 5R | 1S,3S (trans) |
| I-776 | Cl | H | H | CH₂CF₃ | 5(rac) | cis-(rac) |
| I-777 | Cl | H | H | CH₂CF₃ | 5(rac) | trans-(rac) |
| I-778 | Cl | H | H | CH₂CF₃ | 5(rac) | 1R,3S (cis) |
| I-779 | Cl | H | H | CH₂CF₃ | 5(rac) | 1S,3S (trans) |
| I-780 | Cl | H | H | CH₂CF₃ | 5R | 1R,3S (cis) |
| I-781 | Cl | H | H | CH₂CF₃ | 5R | 1S,3S (trans) |
| I-782 | Cl | H | H | CH₂CH₂OMe | 5(rac) | cis-(rac) |
| I-783 | Cl | H | H | CH₂CH₂OMe | 5(rac) | trans-(rac) |
| I-784 | Cl | H | H | CH₂CH₂OMe | 5(rac) | 1R,3S (cis) |
| I-785 | Cl | H | H | CH₂CH₂OMe | 5(rac) | 1S,3S (trans) |
| I-786 | Cl | H | H | CH₂CH₂OMe | 5R | 1R,3S (cis) |
| I-787 | Cl | H | H | CH₂CH₂OMe | 5R | 1S,3S (trans) |
| I-788 | Cl | H | H | CH₂CH₂OEt | 5(rac) | cis-(rac) |
| I-789 | Cl | H | H | CH₂CH₂OEt | 5(rac) | trans-(rac) |
| I-790 | Cl | H | H | CH₂CH₂OEt | 5(rac) | 1R,3S (cis) |
| I-791 | Cl | H | H | CH₂CH₂OEt | 5(rac) | 1S,3S (trans) |
| I-792 | Cl | H | H | CH₂CH₂OEt | 5R | 1R,3S (cis) |
| I-793 | Cl | H | H | CH₂CH₂OEt | 5R | 1S,3S (trans) |
| I-794 | Cl | H | Me | H | 5(rac) | cis-(rac) |
| I-795 | Cl | H | Me | H | 5(rac) | trans-(rac) |
| I-796 | Cl | H | Me | H | 5(rac) | 1R,3S (cis) |
| I-797 | Cl | H | Me | H | 5(rac) | 1S,3S (trans) |
| I-798 | Cl | H | Me | H | 5R | 1R,3S (cis) |
| I-799 | Cl | H | Me | H | 5R | 1S,3S (trans) |
| I-800 | Cl | H | Me | Me | 5(rac) | cis-(rac) |
| I-801 | Cl | H | Me | Me | 5(rac) | trans-(rac) |
| I-802 | Cl | H | Me | Me | 5(rac) | 1R,3S (cis) |
| I-803 | Cl | H | Me | Me | 5(rac) | 1S,3S (trans) |
| I-804 | Cl | H | Me | Me | 5R | 1R,3S (cis) |
| I-805 | Cl | H | Me | Me | 5R | 1S,3S (trans) |
| I-806 | Cl | H | Me | Et | 5(rac) | cis-(rac) |
| I-807 | Cl | H | Me | Et | 5(rac) | trans-(rac) |
| I-808 | Cl | H | Me | Et | 5(rac) | 1R,3S (cis) |
| I-809 | Cl | H | Me | Et | 5(rac) | 1S,3S (trans) |
| I-810 | Cl | H | Me | Et | 5R | 1R,3S (cis) |
| I-811 | Cl | H | Me | Et | 5R | 1S,3S (trans) |
| I-812 | Cl | H | Me | nPr | 5(rac) | cis-(rac) |
| I-813 | Cl | H | Me | nPr | 5(rac) | trans-(rac) |
| I-814 | Cl | H | Me | nPr | 5(rac) | 1R,3S (cis) |
| I-815 | Cl | H | Me | nPr | 5(rac) | 1S,3S (trans) |
| I-816 | Cl | H | Me | nPr | 5R | 1R,3S (cis) |
| I-817 | Cl | H | Me | nPr | 5R | 1S,3S (trans) |
| I-818 | Cl | H | Me | iPr | 5R/S | cis-R/S |
| I-819 | Cl | H | Me | iPr | 5(rac) | cis-(rac) |
| I-820 | Cl | H | Me | iPr | 5(rac) | trans-(rac) |
| I-821 | Cl | H | Me | iPr | 5(rac) | 1R,3S (cis) |
| I-822 | Cl | H | Me | iPr | 5(rac) | 1S,3S (trans) |
| I-823 | Cl | H | Me | iPr | 5R | 1R,3S (cis) |
| I-824 | Cl | H | Me | nBu | 5(rac) | cis-(rac) |
| I-825 | Cl | H | Me | nBu | 5(rac) | trans-(rac) |
| I-826 | Cl | H | Me | nBu | 5(rac) | 1R,3S (cis) |
| I-827 | Cl | H | Me | nBu | 5(rac) | 1S,3S (trans) |
| I-828 | Cl | H | Me | nBu | 5R | 1R,3S (cis) |
| I-829 | Cl | H | Me | nBu | 5R | 1S,3S (trans) |
| I-830 | Cl | H | Me | tBu | 5(rac) | cis-(rac) |
| I-831 | Cl | H | Me | tBu | 5(rac) | trans-(rac) |
| I-832 | Cl | H | Me | tBu | 5(rac) | 1R,3S (cis) |
| I-833 | Cl | H | Me | tBu | 5(rac) | 1S,3S (trans) |
| I-834 | Cl | H | Me | tBu | 5R | 1R,3S (cis) |
| I-835 | Cl | H | Me | tBu | 5R | 1S,3S (trans) |
| I-836 | Cl | H | Me | CH₂CH₂Cl | 5(rac) | cis-(rac) |
| I-837 | Cl | H | Me | CH₂CH₂Cl | 5(rac) | trans-(rac) |
| I-838 | Cl | H | Me | CH₂CH₂Cl | 5(rac) | 1R,3S (cis) |
| I-839 | Cl | H | Me | CH₂CH₂Cl | 5(rac) | 1S,3S (trans) |
| I-840 | Cl | H | Me | CH₂CH₂Cl | 5R | 1R,3S (cis) |
| I-841 | Cl | H | Me | CH₂CH₂Cl | 5R | 1S,3S (trans) |
| I-842 | Cl | H | Me | CH₂CF₃ | 5(rac) | cis-(rac) |
| I-843 | Cl | H | Me | CH₂CF₃ | 5(rac) | trans-(rac) |
| I-844 | Cl | H | Me | CH₂CF₃ | 5(rac) | 1R,3S (cis) |
| I-845 | Cl | H | Me | CH₂CF₃ | 5(rac) | 1S,3S (trans) |
| I-846 | Cl | H | Me | CH₂CF₃ | 5R | 1R,3S (cis) |
| I-847 | Cl | H | Me | CH₂CF₃ | 5R | 1S,3S (trans) |
| I-848 | Cl | H | Me | CH₂CH₂OMe | 5(rac) | cis-(rac) |
| I-849 | Cl | H | Me | CH₂CH₂OMe | 5(rac) | trans-(rac) |
| I-850 | Cl | H | Me | CH₂CH₂OMe | 5(rac) | 1R,3S (cis) |
| I-851 | Cl | H | Me | CH₂CH₂OMe | 5(rac) | 1S,3S (trans) |
| I-852 | Cl | H | Me | CH₂CH₂OMe | 5R | 1R,3S (cis) |
| I-853 | Cl | H | Me | CH₂CH₂OMe | 5R | 1S,3S (trans) |
| I-854 | Cl | H | Me | CH₂CH₂OEt | 5(rac) | cis-(rac) |
| I-855 | Cl | H | Me | CH₂CH₂OEt | 5(rac) | trans-(rac) |
| I-856 | Cl | H | Me | CH₂CH₂OEt | 5(rac) | 1R,3S (cis) |
| I-857 | Cl | H | Me | CH₂CH₂OEt | 5(rac) | 1S,3S (trans) |
| I-858 | Cl | H | Me | CH₂CH₂OEt | 5R | 1R,3S (cis) |
| I-859 | Cl | H | Me | CH₂CH₂OEt | 5R | 1S,3S (trans) |
| I-860 | CN | H | H | H | 5(rac) | cis-(rac) |
| I-861 | CN | H | H | H | 5(rac) | trans-(rac) |
| I-862 | CN | H | H | H | 5(rac) | 1R,3S (cis) |
| I-863 | CN | H | H | H | 5(rac) | 1S,3S (trans) |
| I-864 | CN | H | H | H | 5R | 1R,3S (cis) |
| I-865 | CN | H | H | H | 5R | 1S,3S (trans) |
| I-866 | CN | H | H | Me | 5(rac) | cis-(rac) |
| I-867 | CN | H | H | Me | 5(rac) | trans-(rac) |
| I-868 | CN | H | H | Me | 5(rac) | 1R,3S (cis) |
| I-869 | CN | H | H | Me | 5(rac) | 1S,3S (trans) |
| I-870 | CN | H | H | Me | 5R | 1R,3S (cis) |
| I-871 | CN | H | H | Me | 5R | 1S,3S (trans) |
| I-872 | CN | H | H | Et | 5(rac) | cis-(rac) |
| I-873 | CN | H | H | Et | 5(rac) | trans-(rac) |
| I-874 | CN | H | H | Et | 5(rac) | 1R,3S (cis) |
| I-875 | CN | H | H | Et | 5(rac) | 1S,3S (trans) |
| I-876 | CN | H | H | Et | 5R | 1R,3S (cis) |
| I-877 | CN | H | H | Et | 5R | 1S,3S (trans) |
| I-878 | CN | H | H | nPr | 5(rac) | cis-(rac) |
| I-879 | CN | H | H | nPr | 5(rac) | trans-(rac) |
| I-880 | CN | H | H | nPr | 5(rac) | 1R,3S (cis) |
| I-881 | CN | H | H | nPr | 5(rac) | 1S,3S (trans) |
| I-882 | CN | H | H | nPr | 5R | 1R,3S (cis) |
| I-883 | CN | H | H | nPr | 5R | 1S,3S (trans) |
| I-884 | CN | H | H | iPr | 5R/S | cis-R/S |
| I-885 | CN | H | H | iPr | 5(rac) | cis-(rac) |
| I-886 | CN | H | H | iPr | 5(rac) | trans-(rac) |
| I-887 | CN | H | H | iPr | 5(rac) | 1R,3S (cis) |
| I-888 | CN | H | H | iPr | 5(rac) | 1S,3S (trans) |
| I-889 | CN | H | H | iPr | 5R | 1R,3S (cis) |
| I-890 | CN | H | H | nBu | 5(rac) | cis-(rac) |
| I-891 | CN | H | H | nBu | 5(rac) | trans-(rac) |
| I-892 | CN | H | H | nBu | 5(rac) | 1R,3S (cis) |
| I-893 | CN | H | H | nBu | 5(rac) | 1S,3S (trans) |
| I-894 | CN | H | H | nBu | 5R | 1R,3S (cis) |
| I-895 | CN | H | H | nBu | 5R | 1S,3S (trans) |
| I-896 | CN | H | H | tBu | 5(rac) | cis-(rac) |
| I-897 | CN | H | H | tBu | 5(rac) | trans-(rac) |
| I-898 | CN | H | H | tBu | 5(rac) | 1R,3S (cis) |
| I-899 | CN | H | H | tBu | 5(rac) | 1S,3S (trans) |
| I-900 | CN | H | H | tBu | 5R | 1R,3S (cis) |
| I-901 | CN | H | H | tBu | 5R | 1S,3S (trans) |
| I-902 | CN | H | H | CH₂CH₂Cl | 5(rac) | cis-(rac) |
| I-903 | CN | H | H | CH₂CH₂Cl | 5(rac) | trans-(rac) |
| I-904 | CN | H | H | CH₂CH₂Cl | 5(rac) | 1R,3S (cis) |
| I-905 | CN | H | H | CH₂CH₂Cl | 5(rac) | 1S,3S (trans) |
| I-906 | CN | H | H | CH₂CH₂Cl | 5R | 1R,3S (cis) |
| I-907 | CN | H | H | CH₂CH₂Cl | 5R | 1S,3S (trans) |
| I-908 | CN | H | H | CH₂CF₃ | 5(rac) | cis-(rac) |
| I-909 | CN | H | H | CH₂CF₃ | 5(rac) | trans-(rac) |
| I-910 | CN | H | H | CH₂CF₃ | 5(rac) | 1R,3S (cis) |
| I-911 | CN | H | H | CH₂CF₃ | 5(rac) | 1S,3S (trans) |
| I-912 | CN | H | H | CH₂CF₃ | 5R | 1R,3S (cis) |
| I-913 | CN | H | H | CH₂CF₃ | 5R | 1S,3S (trans) |
| I-914 | CN | H | H | CH₂CH₂OMe | 5(rac) | cis-(rac) |
| I-915 | CN | H | H | CH₂CH₂OMe | 5(rac) | trans-(rac) |
| I-916 | CN | H | H | CH₂CH₂OMe | 5(rac) | 1R,3S (cis) |
| I-917 | CN | H | H | CH₂CH₂OMe | 5(rac) | 1S,3S (trans) |
| I-918 | CN | H | H | CH₂CH₂OMe | 5R | 1R,3S (cis) |
| I-919 | CN | H | H | CH₂CH₂OMe | 5R | 1S,3S (trans) |
| I-920 | CN | H | H | CH₂CH₂OEt | 5(rac) | cis-(rac) |
| I-921 | CN | H | H | CH₂CH₂OEt | 5(rac) | trans-(rac) |
| I-922 | CN | H | H | CH₂CH₂OEt | 5(rac) | 1R,3S (cis) |
| I-923 | CN | H | H | CH₂CH₂OEt | 5(rac) | 1S,3S (trans) |
| I-924 | CN | H | H | CH₂CH₂OEt | 5R | 1R,3S (cis) |
| I-925 | CN | H | H | CH₂CH₂OEt | 5R | 1S,3S (trans) |
| I-926 | Me | H | Me | H | 5(rac) | cis-(rac) |
| I-927 | Me | H | Me | H | 5(rac) | trans-(rac) |
| I-928 | Me | H | Me | H | 5(rac) | 1R,3S (cis) |
| I-929 | Me | H | Me | H | 5(rac) | 1S,3S (trans) |
| I-930 | Me | H | Me | H | 5R | 1R,3S (cis) |
| I-931 | Me | H | Me | H | 5R | 1S,3S (trans) |
| I-932 | Me | H | Me | Me | 5(rac) | cis-(rac) |
| I-933 | Me | H | Me | Me | 5(rac) | trans-(rac) |
| I-934 | Me | H | Me | Me | 5(rac) | 1R,3S (cis) |
| I-935 | Me | H | Me | Me | 5(rac) | 1S,3S (trans) |
| I-936 | Me | H | Me | Me | 5R | 1R,3S (cis) |
| I-937 | Me | H | Me | Me | 5R | 1S,3S (trans) |
| I-938 | Me | H | Me | Et | 5(rac) | cis-(rac) |
| I-939 | Me | H | Me | Et | 5(rac) | trans-(rac) |
| I-940 | Me | H | Me | Et | 5(rac) | 1R,3S (cis) |
| I-941 | Me | H | Me | Et | 5(rac) | 1S,3S (trans) |
| I-942 | Me | H | Me | Et | 5R | 1R,3S (cis) |
| I-943 | Me | H | Me | Et | 5R | 1S,3S (trans) |
| I-944 | Me | H | Me | nPr | 5(rac) | cis-(rac) |
| I-945 | Me | H | Me | nPr | 5(rac) | trans-(rac) |
| I-946 | Me | H | Me | nPr | 5(rac) | 1R,3S (cis) |
| I-947 | Me | H | Me | nPr | 5(rac) | 1S,3S (trans) |
| I-948 | Me | H | Me | nPr | 5R | 1R,3S (cis) |
| I-949 | Me | H | Me | nPr | 5R | 1S,3S (trans) |
| I-950 | Me | H | Me | iPr | 5R/S | cis-R/S |
| I-951 | Me | H | Me | iPr | 5(rac) | cis-(rac) |
| I-952 | Me | H | Me | iPr | 5(rac) | trans-(rac) |
| I-953 | Me | H | Me | iPr | 5(rac) | 1R,3S (cis) |
| I-954 | Me | H | Me | iPr | 5(rac) | 1S,3S (trans) |
| I-955 | Me | H | Me | iPr | 5R | 1R,3S (cis) |
| I-956 | Me | H | Me | nBu | 5(rac) | cis-(rac) |
| I-957 | Me | H | Me | nBu | 5(rac) | trans-(rac) |
| I-958 | Me | H | Me | nBu | 5(rac) | 1R,3S (cis) |
| I-959 | Me | H | Me | nBu | 5(rac) | 1S,3S (trans) |
| I-960 | Me | H | Me | nBu | 5R | 1R,3S (cis) |
| I-961 | Me | H | Me | nBu | 5R | 1S,3S (trans) |
| I-962 | Me | H | Me | tBu | 5(rac) | cis-(rac) |
| I-963 | Me | H | Me | tBu | 5(rac) | trans-(rac) |
| I-964 | Me | H | Me | tBu | 5(rac) | 1R,3S (cis) |
| I-965 | Me | H | Me | tBu | 5(rac) | 1S,3S (trans) |
| I-966 | Me | H | Me | tBu | 5R | 1R,3S (cis) |
| I-967 | Me | H | Me | tBu | 5R | 1S,3S (trans) |
| I-968 | Me | H | Me | CH₂CH₂Cl | 5(rac) | cis-(rac) |
| I-969 | Me | H | Me | CH₂CH₂Cl | 5(rac) | trans-(rac) |
| I-970 | Me | H | Me | CH₂CH₂Cl | 5(rac) | 1R,3S (cis) |
| I-971 | Me | H | Me | CH₂CH₂Cl | 5(rac) | 1S,3S (trans) |
| I-972 | Me | H | Me | CH₂CH₂Cl | 5R | 1R,3S (cis) |
| I-973 | Me | H | Me | CH₂CH₂Cl | 5R | 1S,3S (trans) |
| I-974 | Me | H | Me | CH₂CF₃ | 5(rac) | cis-(rac) |
| I-975 | Me | H | Me | CH₂CF₃ | 5(rac) | trans-(rac) |
| I-976 | Me | H | Me | CH₂CF₃ | 5(rac) | 1R,3S (cis) |
| I-977 | Me | H | Me | CH₂CF₃ | 5(rac) | 1S,3S (trans) |
| I-978 | Me | H | Me | CH₂CF₃ | 5R | 1R,3S (cis) |
| I-979 | Me | H | Me | CH₂CF₃ | 5R | 1S,3S (trans) |
| I-980 | Me | H | Me | CH₂CH₂OMe | 5(rac) | cis-(rac) |
| I-981 | Me | H | Me | CH₂CH₂OMe | 5(rac) | trans-(rac) |
| I-982 | Me | H | Me | CH₂CH₂OMe | 5(rac) | 1R,3S (cis) |
| I-983 | Me | H | Me | CH₂CH₂OMe | 5(rac) | 1S,3S (trans) |
| I-984 | Me | H | Me | CH₂CH₂OMe | 5R | 1R,3S (cis) |
| I-985 | Me | H | Me | CH₂CH₂OMe | 5R | 1S,3S (trans) |
| I-986 | Me | H | Me | CH₂CH₂OEt | 5(rac) | cis-(rac) |
| I-987 | Me | H | Me | CH₂CH₂OEt | 5(rac) | trans-(rac) |
| I-988 | Me | H | Me | CH₂CH₂OEt | 5(rac) | 1R,3S (cis) |
| I-989 | Me | H | Me | CH₂CH₂OEt | 5(rac) | 1S,3S (trans) |
| I-990 | Me | H | Me | CH₂CH₂OEt | 5R | 1R,3S (cis) |
| I-991 | Me | H | Me | CH₂CH₂OEt | 5R | 1S,3S (trans) |
| I-992 | Me | H | CF₃ | H | 5(rac) | cis-(rac) |
| I-993 | Me | H | CF₃ | H | 5(rac) | trans-(rac) |
| I-994 | Me | H | CF₃ | H | 5(rac) | 1R,3S (cis) |
| I-995 | Me | H | CF₃ | H | 5(rac) | 1S,3S (trans) |
| I-996 | Me | H | CF₃ | H | 5R | 1R,3S (cis) |
| I-997 | Me | H | CF₃ | H | 5R | 1S,3S (trans) |
| I-998 | Me | H | CF₃ | Me | 5(rac) | cis-(rac) |
| I-999 | Me | H | CF₃ | Me | 5(rac) | trans-(rac) |
| I-1000 | Me | H | CF₃ | Me | 5(rac) | 1R,3S (cis) |
| I-1OO1 | Me | H | CF₃ | Me | 5(rac) | 1S,3S (trans) |
| I-1002 | Me | H | CF₃ | Me | 5R | 1R,3S (cis) |
| I-1003 | Me | H | CF₃ | Me | 5R | 1S,3S (trans) |
| I-1004 | Me | H | CF₃ | Et | 5(rac) | cis-(rac) |
| I-1005 | Me | H | CF₃ | Et | 5(rac) | trans-(rac) |
| I-1006 | Me | H | CF₃ | Et | 5(rac) | 1R,3S (cis) |
| I-1007 | Me | H | CF₃ | Et | 5(rac) | 1S,3S (trans) |
| I-1008 | Me | H | CF₃ | Et | 5R | 1R,3S (cis) |
| I-1009 | Me | H | CF₃ | Et | 5R | 1S,3S (trans) |
| I-1010 | Me | H | CF₃ | nPr | 5(rac) | cis-(rac) |
| I-1011 | Me | H | CF₃ | nPr | 5(rac) | trans-(rac) |
| I-1012 | Me | H | CF₃ | nPr | 5(rac) | 1R,3S (cis) |
| I-1013 | Me | H | CF₃ | nPr | 5(rac) | 1S,3S (trans) |
| I-1014 | Me | H | CF₃ | nPr | 5R | 1R,3S (cis) |
| I-1015 | Me | H | CF₃ | nPr | 5R | 1S,3S (trans) |
| I-1016 | Me | H | CF₃ | iPr | 5R/S | cis-R/S |
| I-1017 | Me | H | CF₃ | iPr | 5(rac) | cis-(rac) |
| I-1018 | Me | H | CF₃ | iPr | 5(rac) | trans-(rac) |
| I-1019 | Me | H | CF₃ | iPr | 5(rac) | 1R,3S (cis) |
| I-1020 | Me | H | CF₃ | iPr | 5(rac) | 1S,3S (trans) |
| I-1021 | Me | H | CF₃ | iPr | 5R | 1R,3S (cis) |
| I-1022 | Me | H | CF₃ | nBu | 5(rac) | cis-(rac) |
| I-1023 | Me | H | CF₃ | nBu | 5(rac) | trans-(rac) |
| I-1024 | Me | H | CF₃ | nBu | 5(rac) | 1R,3S (cis) |
| I-1025 | Me | H | CF₃ | nBu | 5(rac) | 1S,3S (trans) |
| I-1026 | Me | H | CF₃ | nBu | 5R | 1R,3S (cis) |
| I-1027 | Me | H | CF₃ | nBu | 5R | 1S,3S (trans) |
| I-1028 | Me | H | CF₃ | tBu | 5(rac) | cis-(rac) |
| I-1029 | Me | H | CF₃ | tBu | 5(rac) | trans-(rac) |
| I-1030 | Me | H | CF₃ | tBu | 5(rac) | 1R,3S (cis) |
| I-1031 | Me | H | CF₃ | tBu | 5(rac) | 1S,3S (trans) |
| I-1032 | Me | H | CF₃ | tBu | 5R | 1R,3S (cis) |
| I-1033 | Me | H | CF₃ | tBu | 5R | 1S,3S (trans) |
| I-1034 | Me | H | CF₃ | CH₂CH₂Cl | 5(rac) | cis-(rac) |
| I-1035 | Me | H | CF₃ | CH₂CH₂Cl | 5(rac) | trans-(rac) |
| I-1036 | Me | H | CF₃ | CH₂CH₂Cl | 5(rac) | 1R,3S (cis) |
| I-1037 | Me | H | CF₃ | CH₂CH₂Cl | 5(rac) | 1S,3S (trans) |
| I-1038 | Me | H | CF₃ | CH₂CH₂Cl | 5R | 1R,3S (cis) |
| I-1039 | Me | H | CF₃ | CH₂CH₂Cl | 5R | 1S,3S (trans) |
| I-1040 | Me | H | CF₃ | CH₂CF₃ | 5(rac) | cis-(rac) |
| I-1041 | Me | H | CF₃ | CH₂CF₃ | 5(rac) | trans-(rac) |
| I-1042 | Me | H | CF₃ | CH₂CF₃ | 5(rac) | 1R,3S (cis) |
| I-1043 | Me | H | CF₃ | CH₂CF₃ | 5(rac) | 1S,3S (trans) |
| I-1044 | Me | H | CF₃ | CH₂CF₃ | 5R | 1R,3S (cis) |
| I-1045 | Me | H | CF₃ | CH₂CF₃ | 5R | 1S,3S (trans) |
| I-1046 | Me | H | CF₃ | CH₂CH₂OMe | 5(rac) | cis-(rac) |
| I-1047 | Me | H | CF₃ | CH₂CH₂OMe | 5(rac) | trans-(rac) |
| I-1048 | Me | H | CF₃ | CH₂CH₂OMe | 5(rac) | 1R,3S (cis) |
| I-1049 | Me | H | CF₃ | CH₂CH₂OMe | 5(rac) | 1S,3S (trans) |
| I-1050 | Me | H | CF₃ | CH₂CH₂OMe | 5R | 1R,3S (cis) |
| I-1051 | Me | H | CF₃ | CH₂CH₂OMe | 5R | 1S,3S (trans) |
| I-1052 | Me | H | CF₃ | CH₂CH₂OEt | 5(rac) | cis-(rac) |
| I-1053 | Me | H | CF₃ | CH₂CH₂OEt | 5(rac) | trans-(rac) |
| I-1054 | Me | H | CF₃ | CH₂CH₂OEt | 5(rac) | 1R,3S (cis) |
| I-1055 | Me | H | CF₃ | CH₂CH₂OEt | 5(rac) | 1S,3S (trans) |
| I-1056 | Me | H | CF₃ | CH₂CH₂OEt | 5R | 1R,3S (cis) |
| I-1057 | Me | H | CF₃ | CH₂CH₂OEt | 5R | 1S,3S (trans) |
| I-1058 | OMe | H | OMe | H | 5(rac) | cis-(rac) |
| I-1059 | OMe | H | OMe | H | 5(rac) | trans-(rac) |
| I-1060 | OMe | H | OMe | H | 5(rac) | 1R,3S (cis) |
| I-1061 | OMe | H | OMe | H | 5(rac) | 1S,3S (trans) |
| I-1062 | OMe | H | OMe | H | 5R | 1R,3S (cis) |
| I-1063 | OMe | H | OMe | H | 5R | 1S,3S (trans) |
| I-1064 | OMe | H | OMe | Me | 5(rac) | cis-(rac) |
| I-1065 | OMe | H | OMe | Me | 5(rac) | trans-(rac) |
| I-1066 | OMe | H | OMe | Me | 5(rac) | 1R,3S (cis) |
| I-1067 | OMe | H | OMe | Me | 5(rac) | 1S,3S (trans) |
| I-1068 | OMe | H | OMe | Me | 5R | 1R,3S (cis) |
| I-1069 | OMe | H | OMe | Me | 5R | 1S,3S (trans) |
| I-1070 | OMe | H | OMe | Et | 5(rac) | cis-(rac) |
| I-1071 | OMe | H | OMe | Et | 5(rac) | trans-(rac) |
| I-1072 | OMe | H | OMe | Et | 5(rac) | 1R,3S (cis) |
| I-1073 | OMe | H | OMe | Et | 5(rac) | 1S,3S (trans) |
| I-1074 | OMe | H | OMe | Et | 5R | 1R,3S (cis) |
| I-1075 | OMe | H | OMe | Et | 5R | 1S,3S (trans) |
| I-1076 | OMe | H | OMe | nPr | 5(rac) | cis-(rac) |
| I-1077 | OMe | H | OMe | nPr | 5(rac) | trans-(rac) |
| I-1078 | OMe | H | OMe | nPr | 5(rac) | 1R,3S (cis) |
| I-1079 | OMe | H | OMe | nPr | 5(rac) | 1S,3S (trans) |
| I-1080 | OMe | H | OMe | nPr | 5R | 1R,3S (cis) |
| I-1081 | OMe | H | OMe | nPr | 5R | 1S,3S (trans) |
| I-1082 | OMe | H | OMe | iPr | 5R/S | cis-R/S |
| I-1083 | OMe | H | OMe | iPr | 5(rac) | cis-(rac) |
| I-1084 | OMe | H | OMe | iPr | 5(rac) | trans-(rac) |
| I-1085 | OMe | H | OMe | iPr | 5(rac) | 1R,3S (cis) |
| I-1086 | OMe | H | OMe | iPr | 5(rac) | 1S,3S (trans) |
| I-1087 | OMe | H | OMe | iPr | 5R | 1R,3S (cis) |
| I-1088 | OMe | H | OMe | nBu | 5(rac) | cis-(rac) |
| I-1089 | OMe | H | OMe | nBu | 5(rac) | trans-(rac) |
| I-1090 | OMe | H | OMe | nBu | 5(rac) | 1R,3S (cis) |
| I-1091 | OMe | H | OMe | nBu | 5(rac) | 1S,3S (trans) |
| I-1092 | OMe | H | OMe | nBu | 5R | 1R,3S (cis) |
| I-1093 | OMe | H | OMe | nBu | 5R | 1S,3S (trans) |
| I-1094 | OMe | H | OMe | tBu | 5(rac) | cis-(rac) |
| I-1095 | OMe | H | OMe | tBu | 5(rac) | trans-(rac) |
| I-1096 | OMe | H | OMe | tBu | 5(rac) | 1R,3S (cis) |
| I-1097 | OMe | H | OMe | tBu | 5(rac) | 1S,3S (trans) |
| I-1098 | OMe | H | OMe | tBu | 5R | 1R,3S (cis) |
| I-1099 | OMe | H | OMe | tBu | 5R | 1S,3S (trans) |
| I-1100 | OMe | H | OMe | CH₂CH₂Cl | 5(rac) | cis-(rac) |
| I-1101 | OMe | H | OMe | CH₂CH₂Cl | 5(rac) | trans-(rac) |
| I-1102 | OMe | H | OMe | CH₂CH₂Cl | 5(rac) | 1R,3S (cis) |
| I-1103 | OMe | H | OMe | CH₂CH₂Cl | 5(rac) | 1S,3S (trans) |
| I-1104 | OMe | H | OMe | CH₂CH₂Cl | 5R | 1R,3S (cis) |
| I-1105 | OMe | H | OMe | CH₂CH₂Cl | 5R | 1S,3S (trans) |
| I-1106 | OMe | H | OMe | CH₂CF₃ | 5(rac) | cis-(rac) |
| I-1107 | OMe | H | OMe | CH₂CF₃ | 5(rac) | trans-(rac) |
| I-1108 | OMe | H | OMe | CH₂CF₃ | 5(rac) | 1R,3S (cis) |
| I-1109 | OMe | H | OMe | CH₂CF₃ | 5(rac) | 1S,3S (trans) |
| I-1110 | OMe | H | OMe | CH₂CF₃ | 5R | 1R,3S (cis) |
| I-1111 | OMe | H | OMe | CH₂CF₃ | 5R | 1S,3S (trans) |
| I-1112 | OMe | H | OMe | CH₂CH₂OMe | 5(rac) | cis-(rac) |
| I-1113 | OMe | H | OMe | CH₂CH₂OMe | 5(rac) | trans-(rac) |
| I-1114 | OMe | H | OMe | CH₂CH₂OMe | 5(rac) | 1R,3S (cis) |
| I-1115 | OMe | H | OMe | CH₂CH₂OMe | 5(rac) | 1S,3S (trans) |
| I-1116 | OMe | H | OMe | CH₂CH₂OMe | 5R | 1R,3S (cis) |
| I-1117 | OMe | H | OMe | CH₂CH₂OMe | 5R | 1S,3S (trans) |
| I-1118 | OMe | H | OMe | CH₂CH₂OEt | 5(rac) | cis-(rac) |
| I-1119 | OMe | H | OMe | CH₂CH₂OEt | 5(rac) | trans-(rac) |
| I-1120 | OMe | H | OMe | CH₂CH₂OEt | 5(rac) | 1R,3S (cis) |
| I-1121 | OMe | H | OMe | CH₂CH₂OEt | 5(rac) | 1S,3S (trans) |
| I-1122 | OMe | H | OMe | CH₂CH₂OEt | 5R | 1R,3S (cis) |
| I-1123 | OMe | H | OMe | CH₂CH₂OEt | 5R | 1S,3S (trans) |

In Table 1, the following abbreviations are used:
rac = racemic
Me = methyl
Et = ethyl
nPr = n-propyl
iPr = i-propyl
nBu = n-butyl
tBu = tert-butyl

The compounds according to the invention can be prepared by various processes listed below:

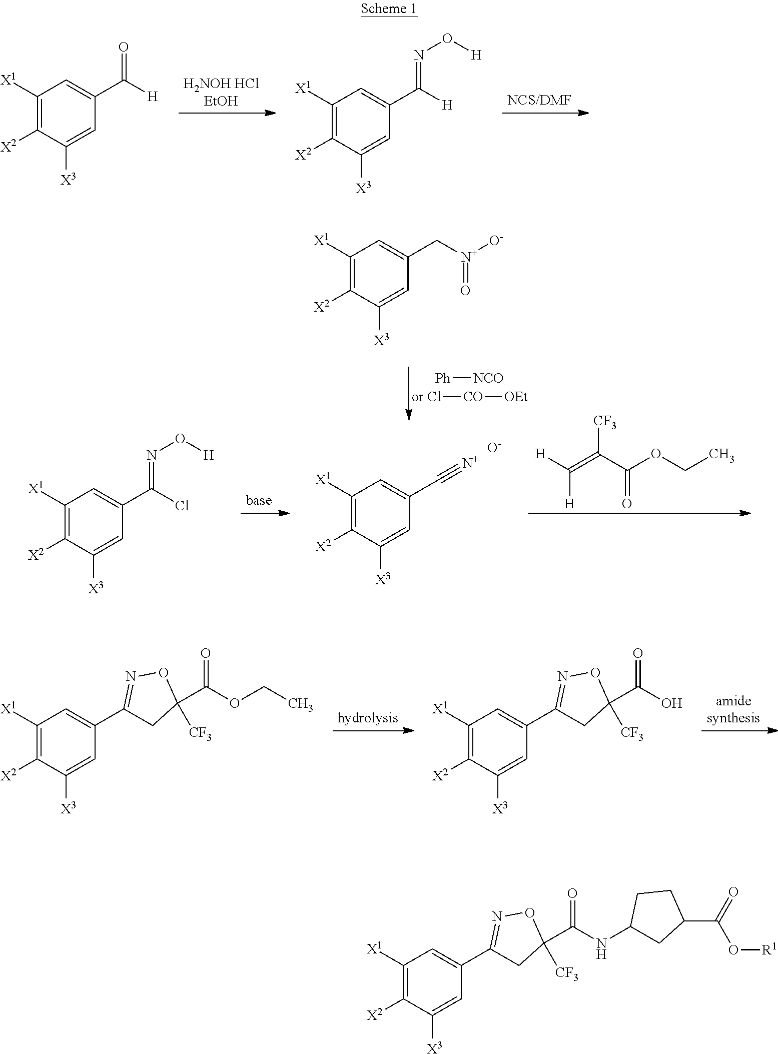

Such reactions shown in Scheme 1 of 1,3-dipolar cycloadditions of nitrile oxides with suitable dipolarophiles are described, for example, in Reviews: 1,3 dipolar Cycloaddition Chemistry, Padwa, ed. Wiley, New York, 1984; Kanemasa and Tsuge, Heterocycles 1990, 30, 719. For the preparation of chloroximes, see Kim, Jae N., Ryu, Eung K. J. Org. Chem. 1992, 57, 6649).

Any diastereoisomer mixtures formed can be separated by column chromatography. Optically active isoxazolines can be obtained by chiral HPLC of suitable precursors or end products, and likewise by enantioselective reactions, for example enzymatic ester or amide cleavage or through the use of chiral auxiliaries on the dipolarophile, as described by Olssen (J. Org. Chem. 1988, 53, 2468).

For preparation of the compounds according to the invention, it is also possible to use suitably substituted 2-alkoxyacrylamides (Scheme 3). These are obtainable from the acrylic esters described in scheme 2 after hydrolysis and amide formation.

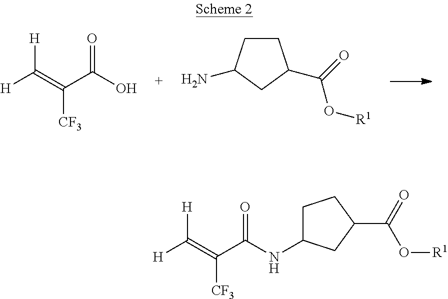

One option for activating the 2-(trifluoromethyl)acrylic acid is carbodiimides, for example EDAC (Chen, F. M. F.; Benoiton, N. L. Synthesis 1979, 709). For preparation of acrylamides, see U.S. Pat. No. 2,521,902, JP60112746, J. of Polymer Science 1979, 17 (6), 1655. Suitably substituted 2-(trifluoromethyl)acrylamides can be reacted in a 1,3-cycloaddition reaction with nitrile oxides to give the compounds according to the invention (Scheme 3).

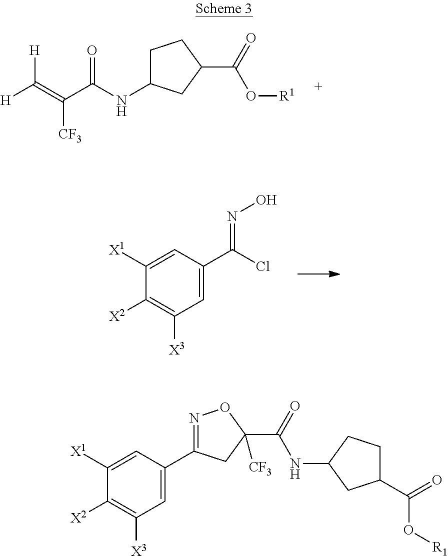

Transformations of the functional groups R¹ are possible either at the alkene stage or at the isoxazoline stage (Scheme 4).

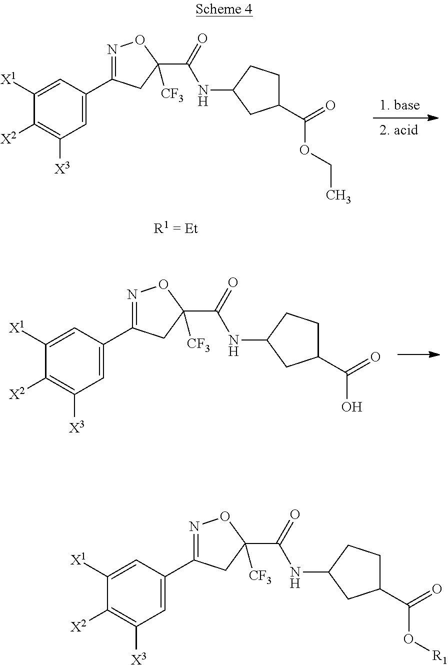

Here, the ethyl ester is cleaved in a known manner by suitable bases, for example aqueous sodium hydroxide solution, and the carboxylic acid is then released by a suitable acid, for example hydrochloric acid. Esterification is then carried out by known methods, for example via the corresponding acid chloride or via activation, for example with carbodiimides such as EDAC.

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the workup or the purification of the products and/or intermediates. Overall, this is understood to mean a procedure as described, for example, by D. Tiebes in Combinatorial Chemistry—Synthesis, Analysis, Screening (editor: Günther Jung), Wiley, 1999, on pages 1 to 34.

The compounds of the formula (I) according to the invention (and/or salts thereof), referred to collectively as “compounds according to the invention” hereinafter, have excellent herbicidal efficacy against a broad spectrum of economically important monocotyledonous and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds according to the invention are applied to the soil surface before germination, either the weed seedlings are prevented completely from emerging or the weeds grow until they have reached the cotyledon stage, but then stop growing.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner

The compounds according to the invention can be selective in crops of useful plants and can also be employed as non-selective herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain active compounds used in agroindustry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the compounds of the formula (I) according to the invention or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A).
genetically modified crop plants having novel constituents or secondary metabolites, for example novel phytoalexins, which cause an increase in disease resistance (EP 0309862 A, EP 0464461 A),
genetically modified plants having reduced photorespiration, which have higher yields and higher stress tolerance (EP 0305398 A),
transgenic crop plants which produce pharmaceutically or diagnostically important proteins (“molecular pharming”),
transgenic crop plants which feature higher yields or better quality,
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To join the DNA fragments with one another, adapters or linkers can be placed onto the fragments, see, for example, Sambrook et al., 1989, Molecular Cloning, A Laboratory Manual, 2nd ed. Cold Spring Harbor Laboratory Press, Cold Spring Harbor, N.Y.; or Winnacker “Gene and Klone” [Genes and Clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Thus, transgenic plants can be obtained whose properties are altered by overexpression, suppression or inhibition of homologous (=natural) genes or gene sequences or expression of heterologous (=foreign) genes or gene sequences.

The compounds (I) according to the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds, or to any desired combinations of these active compounds.

The compounds of the invention can be used with particular preference in transgenic crop plants which are resistant to a combination of glyphosates and glufosinates, glyphosates and sulfonylureas or imidazolinones. Most preferably, the compounds according to the invention can be used in transgenic crop plants such as corn or soya bean with the trade name or the designation Optimum™ GAT™ (glyphosate ALS tolerant), for example

When the active compounds of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds according to the invention of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Küchler, “Chemische Technologic [Chemical Technology]”, Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active Ethylene Oxide Adducts], Wiss. Verlagsgesell., Stuttgart 1976; Winnacker-Küchler, “Chemische Technologic”, volume 7, C. Hanser Verlag Munich, 4th Ed. 1986.

On the basis of these formulations, it is also possible to produce combinations with other active compounds, for example insecticides, acaricides, herbicides, fungicides, and also with safeners, fertilizers and/or growth regulators, for example in the form of a finished formulation or as a tank mix.

Active compounds which can be employed in combination with the compounds according to the invention in mixed formulations or in the tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as are described in, for example, Weed Research 26 (1986) 441-445 or “The Pesticide Manual”, 16th edition, The British Crop Protection Council and the Royal Soc. of Chemistry, 2006 and the literature cited therein. Known herbicides or plant growth regulators which can be combined with the compounds according to the invention are, for example, the following active compounds, where said compounds are designated either with their “common name” in accordance with the International Organization for Standardization (ISO) or with the chemical name or with the code number. They always encompass all of the application forms such as, for example, acids, salts, esters and also all isomeric forms such as stereoisomers and optical isomers, even if they are not explicitly mentioned.

Examples of such herbicidal mixing partners are:

Acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, -isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat dibromide, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]phenyl]ethansulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl) O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron esters, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorophenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and also the following compounds:

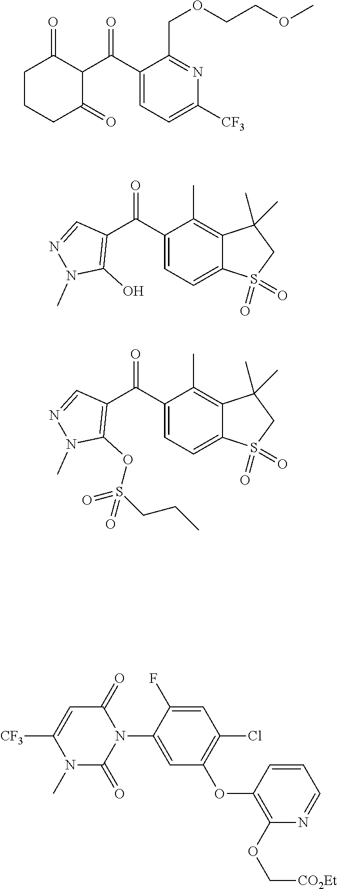 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Safeners which can be employed in combination with the compounds of the formula (I) according to the invention and optionally in combination with further active compounds such as insecticides, acaricides, herbicides, fungicides as listed above are preferably selected from the group consisting of:

S1) Compounds of the formula (S1)

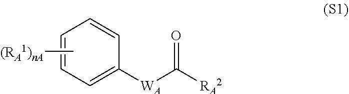

where the symbols and indices are defined as follows:

n_{A} represents a natural number from 0 to 5, preferably from 0 to 3;

R_{A}¹ represents halogen, (C₁-C₄)-alkyl, (C₁-C₄)-alkoxy, nitro or (C₁-C₄)-haloalkyl;

W_{A} represents an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),

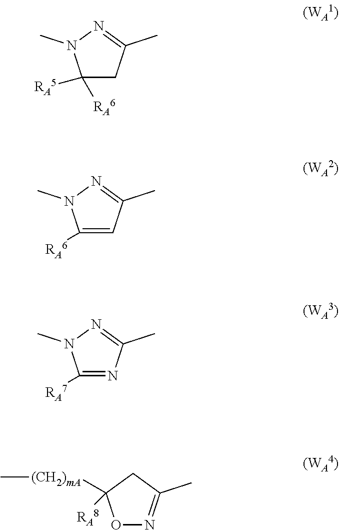

m_{A} represents 0 or 1;

R_{A}² represents OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;

R_{A}³ represents hydrogen or an unsubstituted or substituted aliphatic hydrocarbon radical preferably having a total of 1 to 18 carbon atoms;

R_{A}⁴ represents hydrogen, (C₁-C₆)-alkyl, (C₁-C₆)-alkoxy or substituted or unsubstituted phenyl;

R_{A}⁵ represents H, (C₁-C₈)-alkyl, (C₁-C₈)-haloalkyl, (C₁-C₄)-alkoxy-(C₁-C₈)-alkyl, cyano or COOR_{A}⁹, where R_{A}⁹ represents hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;

R_{A}⁶, R_{A}⁷, R_{A}⁸ are identical or different and represent hydrogen, (C₁-C₈)-alkyl, (C₁-C₈)-haloalkyl, (C₃-C₁₂)-cycloalkyl or substituted or unsubstituted phenyl;

preferably:

a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;

b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;

c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;

d) compounds of the triazolecarboxylic acid type (S1^{d}), preferably compounds such as fenchlorazole (-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;

e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.

S2) Quinoline derivatives of the formula (S2)

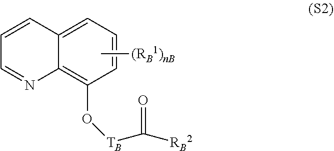

where the symbols and indices are defined as follows:

R_{B}¹ represents halogen, (C₁-C₄)-alkyl, (C₁-C₄)-alkoxy, nitro or (C₁-C₄)-haloalkyl;

n_{B} represents a natural number from 0 to 5, preferably from 0 to 3;

R_{B}² represents OR_{B}³, SR_{B}³ or NR_{B}³R_{B}⁴ or a saturated

or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;

R_{B}³ represents hydrogen or an unsubstituted or substituted aliphatic hydrocarbon radical preferably having a total of 1 to 18 carbon atoms;

R_{B}⁴ represents hydrogen, (C₁-C₆)-alkyl, (C₁-C₆)-alkoxy or substituted or unsubstituted phenyl;

TB represents a (C₁ or C₂)-alkanediyl chain which is unsubstituted or substituted by one or two (C₁-C₄)-alkyl radicals or by [(C₁-C₃)-alkoxy]carbonyl;

preferably:

a) compounds of the 8-quinolinoxyacetic acid type (S2^{a}), preferably

1-methylhexyl (5-chloro-8-quinolinoxy)acetate (“cloquintocet-methyl”) (S2-1), (1,3-dimethylbut-1-yl) (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;

b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.

S3) Compounds of the formula (S3)

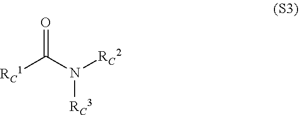

where the symbols and indices are defined as follows:

R_{C}¹ represents (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;

R_{C}², R_{C}³ are identical or different and represent hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;

preferably:
active compounds of the dichloroacetamide type, which are frequently used as pre-emergence safeners (soil-acting safeners), for example

“dichlormid” (N,N-diallyl-2,2-dichloroacetamide) (S3-1),

“R-29148” (3-dichloroacetyl-2,2,5-trimethyl-1,3-oxazolidine) from Stauffer (S3-2),

“R-28725” (3-dichloroacetyl-2,2-dimethyl-1,3-oxazolidine) from Stauffer (S3-3),

“benoxacor” (4-dichloroacetyl-3,4-dihydro-3-methyl-2H-1,4-benzoxazine) (S3-4),

“PPG-1292” (N-allyl-N-[(1,3-dioxolan-2-yl)methyl]dichloroacetamide) from PPG Industries (S3-5),

“DKA-24” (N-allyl-N-[(allylaminocarbonyl)methyl]dichloroacetamide) from Sagro-Chem (S3-6),

“AD-67” or “MON 4660” (3-dichloroacetyl-1-oxa-3-azaspiro[4.5]decane) from Nitrokemia or Monsanto

(S3-7),

“TI-35” (1-dichloroacetylazepane) from TRI-Chemical RT (S3-8),

“diclonon” (dicyclonon) or “BAS145138” or “LAB145138” (S3-9)

((RS)-1-dichloroacetyl-3,3,8a-trimethylperhydropyrrolo[1,2-a]pyrimidin-6-one) from BASF,

“furilazole” or “MON 13900” ((RS)-3-dichloroacetyl-5-(2-furyl)-2,2-dimethyloxazolidine) (S3-10); and the (R) isomer thereof (S3-11).

S4)N-acylsulfonamides of the formula (S4) and salts thereof,

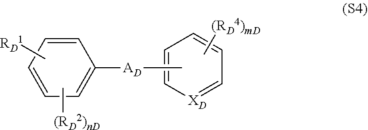

in which the symbols and indices are defined as follows:

A_{D} represents SO₂—NR_{D}²—CO or CO—NR_{D}³—SO₂

X_{D} represents CH or N;

R_{D}¹ represents CO—NR_{D}⁵R_{D}⁶ or NHCO—R_{D}⁷;

R_{D}² represents halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;

R_{D}³ represents hydrogen, (C₁-C₄)-alkyl, (C₂-C₄)-alkenyl or (C₂-C₄)-alkynyl;

R_{D}⁴ represents halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;

R_{D}⁵ represents hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven last-mentioned radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;

R_{D}⁶ represents hydrogen, (C₁-C₆)-alkyl, (C₂-C₆)-alkenyl or (C₂-C₆)-alkynyl, where the three last-mentioned radicals are substituted by v_{D} radicals from the group consisting of halogen, hydroxy, (C₁-C₄)-alkyl, (C₁-C₄)-alkoxy and (C₁-C₄)-alkylthio, or

R_{D}⁵ and R_{D}⁶ together with the nitrogen atom carrying them form a pyrrolidinyl or piperidinyl radical;

R_{D}⁷ represents hydrogen, (C₁-C₄)-alkylamino, (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;

n_{D} represents 0, 1 or 2;

m_{D} represents 1 or 2;

v_{D} represents 0, 1, 2 or 3;

among these, preference is given to compounds of the N-acylsulfonamide type, for example of the formula (S4^{a}) below, which are known, for example, from WO-A-97/45016

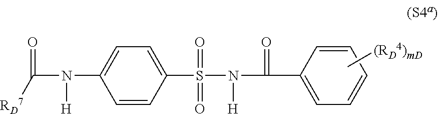

in which

R_{D}⁷ represents (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;

R_{D}⁴ represents halogen, (C₁-C₄)-alkyl, (C₁-C₄)-alkoxy, CF₃;

m_{D} represents 1 or 2;

v_{D} represents 0, 1, 2 or 3;

and also

acylsulfamoylbenzamides, for example of the formula (S4^{b}) below, which are known, for example, from WO-A-99/16744,

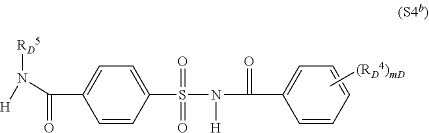

e.g. those in which

R_{D}⁵=cyclopropyl and (R_{D}⁴)=2-OMe (“cyprosulfamide”, S4-1),

R_{D}⁵=cyclopropyl and (R_{D}⁴)=5-Cl-2-OMe (S4-2),

R_{D}⁵=ethyl and (R_{D}⁴)=2-OMe (S4-3),

R_{D}⁵=isopropyl and (R_{D}⁴)=5-Cl-2-OMe (S4-4) and

R_{D}⁵=isopropyl and (R_{D}⁴)=2-OMe (S4-5)

and also

compounds of the N-acylsulfamoylphenylurea type, of the formula (S4^{c}), which are known, for example, from EP-A-365484,

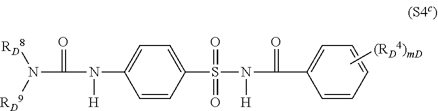

in which

R_{D}⁸ and R_{D}⁹ independently of one another represent hydrogen, (C₁-C₈)-alkyl, (C₃-C₈)-cycloalkyl, (C₃-C₆)-alkenyl, (C₃-C₆)-alkynyl,

R_{D}⁴ represents halogen, (C₁-C₄)-alkyl, (C₁-C₄)-alkoxy, CF₃,

m_{D} represents 1 or 2;

for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,

and also

N-phenylsulfonylterephthalamides of the formula (S4^{d}), which are known, for example, from CN 101838227,

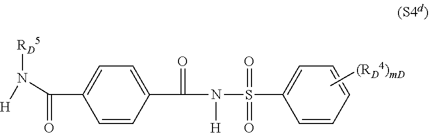

e.g. those in which

R_{D}⁴ represents halogen, (C₁-C₄)-alkyl, (C₁-C₄)-alkoxy, CF₃;

m_{D} represents 1 or 2;

R_{D}⁵ represents hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active compounds from the class of the hydroxyaromatics and the aromatic-aliphatic carboxylic acid derivatives (S5), for example

ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.

S6) Active compounds from the class of the 1,2-dihydroquinoxalin-2-ones (S6), for example

1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

S7) Compounds of the formula (S7), as described in WO-A-1998/38856,

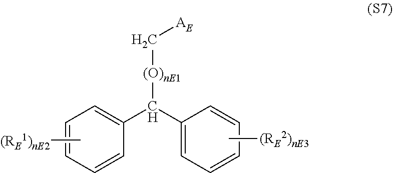

in which the symbols and indices are defined as follows:

R_{E}¹, R_{E}² independently of one another represent halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;

A_{E} represents COOR_{E}³ or COSR_{E}⁴

R_{E}³, R_{E}⁴ independently of one another represent hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,

n_{E}¹ represents 0 or 1

n_{E}², n_{E}³ independently of one another represent 0, 1 or 2,

preferably:

diphenylmethoxyacetic acid,

ethyl diphenylmethoxyacetate,

methyl diphenylmethoxyacetate (CAS reg. no. 41858-19-9) (S7-1).

S8) Compounds of the formula (S8), as described in WO-A-98/27049,

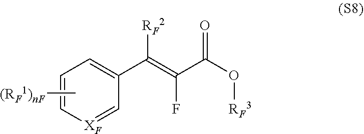

in which

X_{F} represents CH or N,

n_{F} in the case that X_{F}═N is an integer from 0 to 4 and
in the case that X_{F}═CH is an integer from 0 to 5,

R_{F}¹ represents halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,

R_{F}² represents hydrogen or (C₁-C₄)-alkyl,

R_{F}³ represents hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,

preferably compounds in which

X_{F} represents CH,

n_{F} represents an integer from 0 to 2,

R_{F}¹ represents halogen, (C₁-C₄)-alkyl, (C₁-C₄)-haloalkyl, (C₁-C₄)-alkoxy, (C₁-C₄)-haloalkoxy,

R_{F}² represents hydrogen or (C₁-C₄)-alkyl,

R_{F}³ represents hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy,

or salts thereof.

S9) Active compounds from the class of the 3-(5-tetrazolylcarbonyl)-2-quinolones (S9), for example

1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.

S10) Compounds of the formula (S10^{a}) or (S10^{b})
as described in WO-A-2007/023719 and WO-A-2007/023764

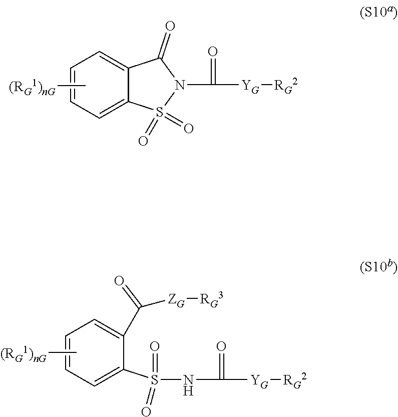

in which

R_{G}¹ represents halogen, (C₁-C₄)-alkyl, methoxy, nitro, cyano, CF₃, OCF₃,

Y_{G}, Z_{G} independently of one another represent O or S,

n_{G} represents an integer from 0 to 4,

R_{G}² represents (C₁-C₁₆)-alkyl, (C₂-C₆)-alkenyl, (C₃-C₆)-cycloalkyl, aryl; benzyl, halobenzyl,

R_{G}³ represents hydrogen or (C₁-C₆)-alkyl.

S11) Active compounds of the oxyimino compound type (S11), which are known as seed-dressing agents, for example

“oxabetrinil” ((Z)-1,3-dioxolan-2-ylmethoxyimino(phenyl)acetonitrile) (S11-1), which is known as a seed-dressing safener for millet/sorghum against metolachlor damage,

“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and

“cyometrinil” or “CGA-43089” ((Z)-cyanomethoxyimino(phenyl)acetonitrile) (S11-3), which is known as a seed-dressing safener for millet/sorghum against metolachlor damage.

S12) Active compounds from the class of the isothiochromanones (S12), for example methyl [(3-oxo-1H-2-benzothiopyran-4(3H)-ylidene)methoxy]acetate (CAS reg. no. 205121-04-6) (S12-1) and related compounds from WO-A-1998/13361.

S13) One or more compounds from group (S13):

“naphthalic anhydride” (1,8-naphthalenedicarboxylic anhydride) (S13-1), which is known as a seed-dressing safener for corn against thiocarbamate herbicide damage,

“fenclorim” (4,6-dichloro-2-phenylpyrimidine) (S13-2), which is known as a safener for pretilachlor in sown rice,

“flurazole” (benzyl 2-chloro-4-trifluoromethyl-1,3-thiazole-5-carboxylate) (S13-3), which is known as a seed-dressing safener for millet/sorghum against alachlor and metolachlor damage,

“CL 304415” (CAS Reg. No. 31541-57-8)

(4-carboxy-3,4-dihydro-2H-1-benzopyran-4-acetic acid) (S13-4) from American Cyanamid, which is known as a safener for corn against damage by imidazolinones,

“MG 191” (CAS Reg. No. 96420-72-3) (2-dichloromethyl-2-methyl-1,3-dioxolane) (S13-5) from Nitrokemia, which is known as a safener for corn,

“MG 838” (CAS Reg. No. 133993-74-5)

(2-propenyl 1-oxa-4-azaspiro[4.5]decane-4-carbodithioate) (S13-6) from Nitrokemia,

“disulfoton” (O,O-diethyl S-2-ethylthioethylphosphorodithioate) (S13-7),

“dietholate” (O,O-diethyl O-phenyl phosphorothioate) (S13-8),

“mephenate” (4-chlorophenyl methylcarbamate) (S13-9).

S14) Active compounds which, in addition to herbicidal action against harmful plants, also have safener action on crop plants such as rice, for example

“dimepiperate” or “MY 93” (S-1-methyl 1-phenylethylpiperidine-1-carbothioate), which is known as a safener for rice against damage by the herbicide molinate,

“daimuron” or “SK 23” (1-(1-methyl-1-phenylethyl)-3-p-tolylurea), which is known as a safener for rice against imazosulfuron herbicide damage,

“cumyluron”=“JC 940” (3-(2-chlorophenylmethyl)-1-(1-methyl-1-phenylethyl)urea, see JP-A-60087254), which is known as safener for rice against damage by some herbicides,

“methoxyphenone” or “NK 049” (3,3′-dimethyl-4-methoxybenzophenone), which is known as a safener for rice against damage by some herbicides,

“CSB” (1-bromo-4-(chloromethylsulfonyl)benzene) from Kumiai, (CAS Reg. No. 54091-06-4), which is known as a safener against damage by some herbicides in rice.

S15) Compounds of the formula (S15) or tautomers thereof

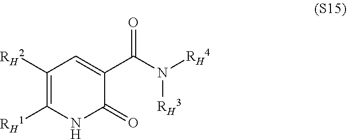

as described in WO-A-2008/131861 and WO-A-2008/131860 in which

R_{H}¹ represents a (C₁-C₆)-haloalkyl radical and

R_{H}² represents hydrogen or halogen and

R_{H}³, V independently of one another represent hydrogen, (C₁-C₁₆)-alkyl, (C₂-C₁₆)-alkenyl or (C₂-C₁₆)-alkynyl,

where each of the 3 last-mentioned radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,

or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,

where each of the 4 last-mentioned radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,

or

R_{H}³ represents (C₁-C₄)-alkoxy, (C₂-C₄)-alkenyloxy, (C₂-C₆)-alkynyloxy or (C₂-C₄)-haloalkoxy and

R_{H}⁴ represents hydrogen or (C₁-C₄)-alkyl or

R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.

S16) Active compounds which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichlor-ethyl).

Particularly preferred safeners are mefenpyr-diethyl, cyprosulfamide, isoxadifen-ethyl, cloquintocet-mexyl and dichlormid.

Wettable powders are preparations uniformly dispersible in water which, in addition to the active compound and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty esters.

Dusting products are obtained by grinding the active compound with finely distributed solids, for example talc, natural clays, such as kaolin, bentonite and pyrophillite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of compounds of the invention. In wettable powders, the active compound concentration is, for example, about 10 to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including, inter alia, temperature, humidity and the type of herbicide used. It can vary within wide limits, for example between 0.001 and 1.0 kg/ha or more of active substance, but it is preferably between 0.005 and 750 g/ha.

A carrier is a natural or synthetic, organic or inorganic substance with which the active compounds are mixed or combined for better applicability, in particular for application to plants or plant parts or seed. The carrier, which may be solid or liquid, is generally inert and should be suitable for use in agriculture. Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, corn cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics and chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions according to the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is necessary if one of the active compounds and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active compounds can be combined with any solid or liquid additive commonly used for formulation purposes. In general, the compositions and formulations according to the invention contain between 0.05 and 99% by weight, 0.01 and 98% by weight, preferably between 0.1 and 95% by weight and more preferably between 0.5 and 90% active compound, most preferably between 10 and 70 percent by weight. The active compounds or compositions according to the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be produced in a manner known per se, for example by mixing the active compounds with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions according to the invention include not only formulations which are already ready for use and can be deployed with a suitable apparatus onto the plant or the seed, but also commercial concentrates which have to be diluted with water prior to use.

The active compounds according to the invention may be present as such or in their (commercial standard) formulations, or else in the use forms prepared from these formulations as a mixture with other (known) active compounds, such as insecticides, attractants, sterilants, bactericides, acaricides, nematicides, fungicides, growth regulators, herbicides, fertilizers, safeners or semiochemicals.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc. It is furthermore possible to apply the active compounds by the ultra-low volume method or to inject the active compound preparation or the active compound itself into the soil.

As also described below, the treatment of transgenic seed with the active compounds according to the invention or compositions is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition according to the invention and/or further additives applied to the seed is chosen such that the germination of the seed is not impaired and the plant which arises therefrom is not damaged. This has to be ensured particularly in the case of active compounds which can exhibit phytotoxic effects at certain application rates.

The compositions according to the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417 A, 4,245,432 A, 4,808,430, 5,876,739, US 2003/0176428 A1, WO 2002/080675 A1, WO 2002/028186 A2.

The active compounds which can be used in accordance with the invention can be converted to the customary seed-dressing formulations, such as solutions, emulsions, suspensions, powders, foams, slurries or other coating compositions for seed, and also ULV formulations.

These formulations are produced in a known manner, by mixing the active compounds with customary additives, for example customary extenders and solvents or diluents, dyes, wetting agents, dispersants, emulsifiers, antifoams, preservatives, secondary thickeners, adhesives, gibberellins, and also water. Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active compounds. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active compounds. Preference is given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ether, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active compounds. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active compounds according to the invention, given good plant compatibility, favorable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

Plants which can be treated in accordance with the invention include the following main crop plants: corn, soya bean, cotton, Brassica oil seeds such as Brassica napus (e.g. Canola), Brassica rapa, B. juncea (e.g. (field) mustard) and Brassica carinata, rice, wheat, sugar beet, sugar cane, oats, rye, barley, millet and sorghum, triticale, flax, grapes and various fruit and vegetables from various botanic taxa, for example Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes, potatoes, peppers, eggplants), Liliaceae sp., Compositae sp. (for example lettuce, artichokes and chicory—including root chicory, endive or common chicory), Umbelliferae sp. (for example carrots, parsley, celery and celeriac), Cucurbitaceae sp. (for example cucumbers—including gherkins, pumpkins, watermelons, calabashes and melons), Alliaceae sp. (for example leeks and onions), Cruciferae sp. (for example white cabbage, red cabbage, broccoli, cauliflower, Brussels sprouts, pak choi, kohlrabi, radishes, horseradish, cress and chinese cabbage), Leguminosae sp. (for example peanuts, peas, and beans—for example common beans and broad beans), Chenopodiaceae sp. (for example Swiss chard, fodder beet, spinach, beetroot), Malvaceae (for example okra), Asparagaceae (for example asparagus); useful plants and ornamental plants in the garden and woods; and in each case genetically modified types of these plants.

As mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) which have been grown by conventional breeding, by mutagenesis or by recombinant DNA techniques. They may be cultivars, varieties, biotypes or genotypes.

The treatment method according to the invention can be used for the treatment of genetically modified organisms (GMOs), e.g. plants or seeds. Genetically modified plants (or transgenic plants) are plants in which a heterologous gene has been stably integrated into the genome. The term “heterologous gene” means essentially a gene which is provided or assembled outside the plant and which, upon introduction into the nuclear genome, the chloroplast genome or the mitochondrial genome, imparts to the transformed plant novel or improved agronomical or other traits because it expresses a protein or polypeptide of interest or another gene which is present in the plant, or other genes which are present in the plant are down-regulated or switched off (for example by means of antisense technology, cosuppression technologies or RNAi technologies [RNA interference]). A heterologous gene that is located in the genome is also called a transgene. A transgene that is defined by its specific presence in the plant genome is called a transformation or transgenic event.

Depending on the plant species or plant cultivars, their location and growth conditions (soils, climate, vegetation period, diet), the inventive treatment may also result in superadditive (“synergistic”) effects. For example, the following effects which exceed the effects actually to be expected are possible: reduced application rates and/or widened spectrum of activity and/or increased efficacy of the active ingredients and compositions which can be used in accordance with the invention, better plant growth, increased tolerance to high or low temperatures, increased tolerance to drought or to water or soil salinity, increased flowering performance, easier harvesting, accelerated maturation, higher harvest yields, bigger fruits, greater plant height, greener leaf color, earlier flowering, higher quality and/or a higher nutritional value of the harvested products, higher sugar concentration within the fruits, better storage stability and/or processability of the harvested products.

Plants and plant cultivars which are preferably treated in accordance with the invention include all plants which have genetic material which imparts particularly advantageous, useful traits to these plants (whether obtained by breeding and/or biotechnological means).

Examples of nematode-resistant plants are described, for example, in the following U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 and 12/497,221.

Plants that may be treated according to the invention are hybrid plants that already express the characteristics of heterosis, or hybrid effect, which results in generally higher yield, vigor, better health and resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate by various methods. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science, 221, 370-371), the CP4 gene of the bacterium Agrobacterium sp. (Barry et al., 1992, Curr. Topics Plant Physiol. 7, 139-145), the genes encoding a petunia EPSPS (Shah et al., 1986, Science 233, 478-481), a tomato EPSPS (Gasser et al., 1988, J. Biol. Chem. 263, 4280-4289) or an Eleusine EPSPS (WO 01/66704). It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes. Plants which express EPSPS genes which impart glyphosate tolerance have been described. Plants which express other genes which impart glyphosate tolerance, for example decarboxylase genes, have been described.

Other herbicide-resistant plants are for example plants made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyze the reaction in which para-hydroxyphenylpyruvate (HPP) is converted to homogentisate. Plants tolerant to HPPD inhibitors can be transformed with a gene encoding a naturally-occurring resistant HPPD enzyme, or a gene encoding a mutated or chimeric HPPD enzyme, as described in WO 96/38567, WO 99/24585, WO 99/24586, WO 2009/144079, WO 2002/046387 or U.S. Pat. No. 6,768,044. Tolerance to HPPD inhibitors can also be obtained by transforming plants with genes encoding certain enzymes enabling the formation of homogentisate despite inhibition of the native HPPD enzyme by the HPPD inhibitor. Such plants are described in WO 99/34008 and WO 02/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928. In addition, plants can be made more tolerant to HPPD inhibitors by inserting into the genome thereof a gene which encodes an enzyme which metabolizes or degrades HPPD inhibitors, for example CYP450 enzymes (see WO 2007/103567 and WO 2008/150473).

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright (Weed Science 2002, 50, 700-712). The production of sulfonylurea-tolerant plants and imidazolinone-tolerant plants has been described. Further sulfonylurea- and imidazolinone-tolerant plants have also been described.

Further plants tolerant to imidazolinones and/or sulfonylureas can be obtained by induced mutagenesis, by selection in cell cultures in the presence of the herbicide or by mutation breeding (cf., for example, for soya beans U.S. Pat. No. 5,084,082, for rice WO 97/41218, for sugar beet U.S. Pat. No. 5,773,702 and WO 99/057965, for lettuce U.S. Pat. No. 5,198,599 or for sunflower WO 01/065922).

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose) polymerase (PARP) gene in the plant cells or plants;

b. plants which contain a stress tolerance-enhancing transgene capable of reducing the expression and/or the activity of the PARG-encoding genes of the plants or plant cells;

c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase. Plants or plant cultivars (obtained by plant biotechnology methods such as genetic engineering) which may also be treated according to the invention show altered quantity, quality and/or storage stability of the harvested product and/or altered properties of specific components of the harvested product such as, for example:

1) Transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications.

2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.

3) Transgenic plants which produce hyaluronan.

4) Transgenic plants or hybrid plants such as onions with particular properties, such as “high soluble solids content”, “low pungency” (LP) and/or “long storage” (LS).

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:

a) plants, such as cotton plants, containing an altered form of cellulose synthase genes;

b) plants, such as cotton plants, which contain an altered form of rsw2 or rsw3 homologous nucleic acids, such as cotton plants with an increased expression of sucrose phosphate synthase;

c) plants, such as cotton plants, with increased expression of sucrose synthase;

d) plants, such as cotton plants, wherein the timing of the plasmodesmatal gating at the base of the fiber cell is altered, for example through downregulation of fiber-selective β-1,3-glucanase;

e) plants, such as cotton plants, which have fibers with altered reactivity, for example through expression of the N-acetylglucosaminetransferase gene, including nodC, and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content;

b) plants, such as oilseed rape plants, which produce oil having a low linolenic acid content;

c) plants, such as oilseed rape plants, which produce oil having a low level of saturated fatty acids.

Plants or plant cultivars (which can be obtained by plant biotechnology methods such as genetic engineering) which may also be treated according to the invention are plants such as potatoes which are virus-resistant, for example to the potato virus Y (SY230 and SY233 events from Tecnoplant, Argentina), or which are resistant to diseases such as potato late blight (e.g. RB gene), or which exhibit reduced cold-induced sweetness (which bear the genes Nt-Inh, II-INV) or which exhibit the dwarf phenotype (A-20 oxidase gene).

Plants or plant cultivars (obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants containing a mutation imparting such altered characteristics, and include plants such as oilseed rape with retarded or reduced seed shattering.

Particularly useful transgenic plants which can be treated according to the invention are plants with transformation events or combinations of transformation events which are the subject of granted or pending petitions for nonregulated status in the USA at the Animal and Plant Health Inspection Service (APHIS) of the United States Department of Agriculture (USDA). Information relating to this is available at any time from APHIS (4700 River Road Riverdale, Md. 20737, USA), for example via the website http://www.aphis.usda.gov/brs/not_reg.html. At the filing date of this application, the petitions with the following information were either granted or pending at the APHIS:
Petition: Identification number of the petition. The technical description of the transformation event can be found in the specific petition document available from APHIS on the website via the petition number. These descriptions are hereby disclosed by reference.
Extension of a petition: Reference to an earlier petition for which an extension of scope or term is being requested.
Institution: Name of the person submitting the petition.
Regulated article: The plant species in question.
Transgenic phenotype: The trait imparted to the plant by the transformation event.
Transformation event or line: The name of the event(s) (sometimes also referred to as line(s)) for which nonregulated status is being requested.
APHIS documents: Various documents which have been published by APHIS with regard to the petition or can be obtained from APHIS on request.

Particularly useful transgenic plants which can be treated in accordance with the invention are plants which comprise one or more genes which code for one or more toxins, for example the transgenic plants which are sold under the following trade names: YIELD GARD® (for example corn, cotton, soya beans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants include corn varieties, cotton varieties and soya bean varieties which are available under the following trade names: Roundup Ready® (tolerance to glyphosates, for example corn, cotton, soya beans), Liberty Link® (tolerance to phosphinothricin, for example oilseed rape), IMI® (tolerance to imidazolinone) and SCS® (tolerance to sulfonylurea), for example corn. Herbicide-resistant plants (plants bred in a conventional manner for herbicide tolerance) which may be mentioned include the varieties sold under the name Clearfield® (for example corn).

Particularly useful transgenic plants which may be treated according to the invention are plants containing transformation events, or a combination of transformation events, and that are listed for example in the databases for various national or regional regulatory agencies (see for example http://gmoinfajrcit/gmp_browse.aspx and http://cera-gmc.org/index.php?evidcode=&hstIDXCode=&gType=&AbbrCode=&atCode=&stCode=&coIDCode=&action=gm_crop_database&mode=Submit).

### A. CHEMICAL EXAMPLES

The NMR data of disclosed examples are listed either in conventional form (δ values, number of hydrogen atoms, multiplet splitting) or as so-called NMR peak lists. In the NMR peak list method, the NMR data of selected examples are recorded in the form of NMR peak lists, where for each signal peak first the δ value in ppm and then, separated by a space, the signal intensity are listed. The δ value/signal intensity number pairs for different signal peaks are listed with separation from one another by semicolons.

The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

Calibration of the chemical shift of ¹H NMR spectra is accomplished using tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of ¹H NMR peak lists can be found in the Research Disclosure Database Number 564025.

The examples which follow illustrate the invention in detail.

### Intermediate 1

### Preparation of 3,5-difluoro-N-hydroxybenzenecarboximidoyl Chloride

Analogously to the procedure in WO2012/130798 for 3,5-dichloro-N-hydroxybenzenecarboximidoyl chloride, 3,5-difluoro-N-hydroxybenzenecarboximidoyl chloride was prepared from 3,5-difluorobenzaldehyde in two steps.

### Intermediate 2

### Preparation of methyl 3-(3,5-difluorophenyl)-5-(trifluoromethyl)-4,5-dihydro-1,2-oxazole-5-carboxylate

Analogously to the procedure in WO2012/130798 for methyl 3-(3,5-difluorophenyl)-5-methyl-4,5-dihydro-1,2-oxazole-5-carboxylate, methyl 3-(3,5-difluorophenyl)-5-(trifluoromethyl)-4,5-dihydro-1,2-oxazole-5-carboxylate was prepared from 3,5-difluorobenzaldehyde in three steps.

### Intermediate 3

### Preparation of 3-(3,5-difluorophenyl)-5-(trifluoromethyl)-4,5-dihydro-1,2-oxazole-5-carboxylic Acid

Analogously to the procedure in WO2012/130798 for 3-(3,5-dichlorophenyl)-5-methyl-4,5-dihydro-1,2-oxazole-5-carboxylic acid, 3-(3,5-difluorophenyl)-5-(trifluoromethyl)-4,5-dihydro-1,2-oxazole-5-carboxylic acid was prepared by hydrolysis of methyl 3-(3,5-difluorophenyl)-5-(trifluoromethyl)-4,5-dihydro-1,2-oxazole-5-carboxylate.

### Intermediate 4

### Preparation of 3-(3,5-difluorophenyl)-5-(trifluoromethyl)-4,5-dihydro-1,2-oxazole-5-carbonyl Chloride

Analogously to the procedure in WO2012/130798 for N-tert-butyl-3-(3,5-dichlorophenyl)-5-methyl-4,5-dihydro-1,2-oxazole-5-carboxamide, 3-(3,5-difluorophenyl)-5-(trifluoromethyl)-4,5-dihydro-1,2-oxazole-5-carbonyl chloride was prepared from 3-(3,5-difluorophenyl)-5-(trifluoromethyl)-4,5-dihydro-1,2-oxazole-5-carboxylic acid by reaction with oxalyl chloride and used as crude product without further purification.

### Example I-11

### Preparation of (1R,3S)-3-({[(5R)-3-(3,5-difluorophenyl)-5-(trifluoromethyl)-4,5-dihydro-1,2-oxazol-5-yl]carbonyl}amino)cyclopentanecarboxylic Acid

1.0 g (2.30 mmol) of ethyl (1R,3S)-3-({[(5R)-3-(3,5-difluorophenyl)-5-(trifluoromethyl)-4,5-dihydro-1,2-oxazol-5-yl]carbonyl}amino)cyclopentanecarboxylate (1-39) were dissolved in 10 ml of tetrahydrofuran and 2 ml of water. 144.9 mg (3.45 mmol) of LiOH×H₂O were then added and the mixture was stirred at 50° C. for 5 h. For work-up, the mixture was concentrated on a rotary evaporator and 50 ml of dichloromethane and 20 ml of 2 M HCl were added to the residue. The organic phase was separated off and the aqueous phase was extracted two more times, in each case with 20 ml of dichloromethane. The combined organic phases were dried over sodium sulfate and concentrated under reduced pressure. Chromatography of the evaporation residue on silica gel (mobile phase n-heptane/ethyl acetate) gave 0.92 g (98%) of (1R,3S)-3-({[(5R)-3-(3,5-difluorophenyl)-5-(trifluoromethyl)-4,5-dihydro-1,2-oxazol-5-yl]carbonyl}amino)cyclopentanecarboxylic acid.

¹H-NMR (CDCl₃): δ 1.78-2.23 (m, 6H), 2.99 (m, 1H), 3.72 (d, 1H), 3.96 (d, 1H), 4.38 (m, 1H), 6.93 (m, 1H), 7.26 (m, 2H), 7.54 (d, br, 1H)

### Example I-20

### Preparation of methyl (1S,3S)-3-({[3-(3,5-difluorophenyl)-5-(trifluoromethyl)-4,5-dihydro-1,2-oxazol-5-yl]carbonyl}amino)cyclopentanecarboxylate

133.6 mg (1.32 ml) of triethylamine were added to 65.2 mg (0.36 mmol) of methyl (1R,3S)-3-aminocyclopentanecarboxylate hydrochloride in 10 ml of dichloromethane, 65.2 mg (0.36 mmol) of carbonyl chloride (intermediate 4) in 10 ml of dichloromethane were added at 0° C., the mixture was stirred for 18 h warming to room temperature and finally water was added. The organic phase was removed, dried with sodium sulfate and concentrated under reduced pressure. Chromatography of the evaporation residue on silica gel (mobile phase n-heptane/ethyl acetate) gave 84 mg (61%) of methyl (1S,3S)-3-({[3-(3,5-difluorophenyl)-5-(trifluoromethyl)-4,5-dihydro-1,2-oxazol-5-yl]carbonyl}amino)cyclopentanecarboxylate as a diastereomer mixture.

¹H-NMR (CDCl₃): δ 1.55 (m, 1H), 1.78 (m, 1H), 1.88 (m, 1H), 2.08 (m, 1H), 2.20 (m, 1H), 2.30 (m, 1H), 2.95 (m, 1H), 3.68 and 3.70 (s, 3H), 3.72 (d, 1H), 3.97 (d, 1H), 4.34 (m, 1H), 6.72 (d, br, 1H), 6.94 (m, 1H), 7.19 (m, 2H)

### Example I-85

### Preparation of Tert-Butyl cis-3-({[3-(3,5-difluorophenyl)-5-(trifluoromethyl)-4,5-dihydro-1,2-oxazol-5-yl]carbonyl}amino)cyclopentanecarboxylate

115.1 mg (0.28 mmol) of cis-3-({[3-(3,5-difluorophenyl)-5-(trifluoromethyl)-4,5-dihydro-1,2-oxazol-5-yl]carbonyl}amino)cyclopentanecarboxylic acid and 25.2 mg (0.34 mmol) of tert-butanol were dissolved in 20 ml of dichloromethane, and 87.8 mg (0.43 mmol) of 1,3-dicyclohexylcarbodiimide and 3.5 mg (0.03 mmol) of 4-dimethylaminopyridine were added. The mixture was left to stir at room temperature overnight. 50 ml of tert-butyl methyl ether were added and the mixture was filtered. The filtrate was concentrated under reduced pressure and the residue was purified by chromatography on silica gel (mobile phase n-heptane/ethyl acetate). This gave 61 mg (40%) of tert-butyl cis-3-({[3-(3,5-difluorophenyl)-5-(trifluoromethyl)-4,5-dihydro-1,2-oxazol-5-yl]carbonyl}amino)cyclopentanecarboxylate as a diastereomer mixture.

¹H-NMR (CDCl₃): δ 1.45 and 1.47 (s, 9H), 1.58-2.26 (m, 6H), 2.82 (m, 1H), 3.71 and 3.72 (d, 1H), 3.96 and 4.00 (d, 1H), 4.35 (m, 1H), 6.92 (m, 1H), 7.20 (m, 2H), 7.49 and 7.51 (d, br, 1H)

### Example I-109

### Preparation of 2-chloroethyl-(1R,3S)-3-({[(5R)-3-(3,5-difluorophenyl)-5-(trifluoromethyl)-4,5-dihydro-1,2-oxazol-5-yl]carbonyl}amino)cyclopentanecarboxylate

130 mg (0.32 mmol) of (1R,3S)-3-({[(5R)-3-(3,5-difluorophenyl)-5-(trifluoromethyl)-4,5-dihydro-1,2-oxazol-5-yl]carbonyl}amino)cyclopentanecarboxylic acid were dissolved in 20 ml of dichloromethane, and 60.9 mg (0.48 mmol) of oxalyl chloride were added. The mixture was left to stir at room temperature for 3 h and then concentrated under reduced pressure, and the residue was re-dissolved in 20 ml of dichloromethane. 51.5 mg (0.64 mmol) of 2-chloroethanol together with 129.5 mg (1.28 mmol) of triethylamine were initially charged in 20 ml of dichloromethane, and the acid chloride, dissolved in dichloromethane, was then added dropwise with stirring. The mixture was left to stir overnight, 20 ml of 2 M HCl were added and the organic phase was then separated off. The aqueous phase was extracted twice more with 20 ml of dichloromethane each time. The combined organic phases were dried over sodium sulfate and concentrated under reduced pressure. Chromatography of the evaporation residue on silica gel (mobile phase n-heptane/ethyl acetate) gave 106.0 mg (71%) of 2-chloroethyl-(1R,3S)-3-({[(5R)-3-(3,5-difluorophenyl)-5-(trifluoromethyl)-4,5-dihydro-1,2-oxazol-5-yl]carbonyl}amino)cyclopentanecarboxylate.

¹H-NMR (CDCl₃): δ 1.78-2.22 (m, 6H), 2.99 (m, 1H), 3.70 (m, 2H), 3.72 (d, 1H), 3.97 (d, 1H), 4.38 (m, 3H), 6.92 (m, 1H), 7.20 (m, 2H), 7.50 (d, br, 1H)

In analogy to the preparation examples cited above and cited at the appropriate point, and taking account of the general details relating to the preparation of substituted isoxazolinecarboxamides, the other compounds listed in Table 1 are obtained:

Analytical data of further examples:

I-28: 1H-NMR (400.0 MHz, CDCl3):

δ=7.2703 (0.5); 7.2687 (0.6); 7.2596 (80.8); 7.2021 (1.2); 7.1990 (0.8); 7.1964 (1.4); 7.1932 (0.8); 7.1860 (0.8); 7.1829 (1.4); 7.1771 (1.2); 6.9670 (0.5); 6.9512 (0.6); 6.9455 (1.1); 6.9397 (0.5); 6.9239 (0.5); 3.9915 (0.8); 3.9454 (1.1); 3.7497 (2.3); 3.7045 (1.6); 3.6774 (16.0); 2.9479 (0.6); 2.3181 (0.5); 2.3010 (0.5); 2.2836 (0.6); 1.9204 (0.6); 1.9012 (0.6); 1.5352 (4.1); 0.0079 (1.1); -0.0002 (35.2); -0.0085 (1.0)

I-29: 1H-NMR (400.0 MHz, CDCl3):

δ=7.6250 (0.8); 7.6061 (0.8); 7.5025 (0.7); 7.4839 (0.7); 7.2622 (34.7); 7.2271 (0.6); 7.2209 (0.7); 7.2145 (2.6); 7.2084 (4.8); 7.2050 (3.7); 7.2026 (3.7); 7.1987 (3.5); 7.1947 (3.7); 7.1928 (3.7); 7.1890 (4.8); 7.1830 (2.7); 7.1766 (0.8); 7.1704 (0.6); 6.9562 (0.8); 6.9506 (1.4); 6.9469 (0.8); 6.9346 (1.6); 6.9291 (2.7); 6.9255 (1.5); 6.9130 (0.8); 6.9074 (1.4); 6.9039 (0.7); 5.2995 (3.6); 4.4089 (0.8); 4.3950 (0.9); 4.3840 (0.8); 4.3761 (0.9); 4.3651 (1.0); 4.3520 (0.8); 4.3434 (0.5); 4.2209 (2.1); 4.2032 (7.6); 4.1855 (10.4); 4.1683 (6.6); 4.1509 (1.8); 4.0057 (2.0); 3.9930 (2.0); 3.9607 (2.8); 3.9479 (2.8); 3.7378 (4.7); 3.7344 (5.2); 3.6927 (3.4); 3.6894 (3.8); 2.9418 (0.7); 2.9308 (1.3); 2.9191 (1.7); 2.9083 (1.6); 2.8973 (1.3); 2.8880 (0.6); 2.2770 (0.7); 2.2592 (0.8); 2.2547 (0.8); 2.2423 (1.0); 2.2370 (0.8); 2.2245 (1.0); 2.2201 (1.0); 2.2022 (0.8); 2.1695 (0.7); 2.1523 (0.8); 2.1466 (0.8); 2.1346 (1.0); 2.1295 (0.9); 2.1174 (1.0); 2.1117 (1.0); 2.0948 (1.2); 2.0841 (0.9); 2.0726 (0.7); 2.0615 (0.8); 2.0547 (1.1); 2.0308 (1.0); 2.0042 (1.0); 1.9897 (2.5); 1.9794 (1.5); 1.9715 (3.9); 1.9563 (2.4); 1.9532 (2.5); 1.9471 (2.2); 1.9402 (2.0); 1.9320 (1.9); 1.9196 (0.9); 1.9111 (0.9); 1.8990 (1.6); 1.8882 (1.3); 1.8761 (1.7); 1.8637 (1.5); 1.8540 (1.0); 1.8418 (1.0); 1.8290 (0.6); 1.8224 (0.6); 1.8104 (0.6); 1.7920 (0.8); 1.7793 (0.9); 1.7707 (0.5); 1.6962 (0.5); 1.6826 (1.0); 1.6666 (0.7); 1.3074 (6.8); 1.2897 (16.0); 1.2734 (15.4); 1.2558 (6.8); -0.0002 (13.0); -0.0083 (0.5)

I-42: 1H-NMR (400.0 MHz, CDCl3):

δ=7.2599 (59.0); 7.1970 (0.6); 7.1835 (0.6); 7.1778 (0.5); 4.1369 (1.6); 4.1190 (1.6); 3.7495 (0.9); 3.7043 (0.7); 1.5392 (16.0); 1.2652 (1.8); 1.2474 (3.7); 1.2296 (1.7); 0.0080 (0.7); -0.0002 (24.0); -0.0085 (0.7)

I-39: 1H-NMR (400.0 MHz, CDCl3):

δ=7.6224 (1.3); 7.6048 (1.2); 7.2605 (42.0); 7.2259 (0.6); 7.2127 (3.7); 7.2077 (4.2); 7.1935 (4.3); 7.1757 (0.5); 6.9557 (0.8); 6.9502 (1.3); 6.9445 (0.7); 6.9340 (1.6); 6.9286 (2.5); 6.9229 (1.3); 6.9126 (0.8); 6.9071 (1.3); 6.9014 (0.6); 4.3829 (0.7); 4.3652 (1.3); 4.3525 (1.3); 4.2046 (2.2); 4.1867 (6.7); 4.1689 (6.8); 4.1510 (2.3); 4.0048 (3.0); 3.9598 (4.1); 3.7322 (5.5); 3.6872 (4.0); 3.4907 (1.1); 2.9436 (0.8); 2.9326 (1.2); 2.9206 (1.5); 2.9074 (1.3); 2.8978 (0.8); 2.1695 (1.0); 2.1518 (1.1); 2.1466 (1.0); 2.1342 (1.5); 2.1293 (1.2); 2.1169 (1.5); 2.1117 (1.4); 2.0951 (1.6); 2.0837 (1.4); 2.0728 (1.0); 2.0612 (1.2); 2.0544 (1.5); 2.0307 (1.2); 2.0049 (1.7); 1.9881 (0.9); 1.9698 (1.2); 1.9561 (2.2); 1.9463 (2.4); 1.9322 (2.0); 1.9105 (1.4); 1.8996 (2.5); 1.8875 (1.2); 1.8746 (0.8); 1.8638 (1.4); 1.8536 (0.8); 1.8219 (0.8); 1.8098 (1.0); 1.7917 (1.2); 1.7800 (1.4); 1.5527 (3.6); 1.2913 (8.1); 1.2734 (16.0); 1.2556 (7.8); -0.0002 (17.1)

I-53: 1H-NMR (400.0 MHz, CDCl3):

δ=7.2598 (68.4); 7.2123 (1.3); 7.2095 (1.1); 7.2066 (1.6); 7.2032 (1.1); 7.1967 (1.0); 7.1930 (1.6); 7.1872 (1.4); 6.9498 (0.6); 6.9340 (0.7); 6.9282 (1.1); 6.9225 (0.7); 6.9066 (0.6); 4.0950 (1.0); 4.0870 (1.1); 4.0781 (2.0); 4.0700 (2.1); 4.0611 (1.2); 4.0532 (1.1); 4.0327 (0.8); 4.0003 (0.9); 3.9552 (1.3); 3.7316 (2.3); 3.6865 (1.8); 2.1388 (0.5); 2.1216 (0.5); 2.0985 (0.6); 2.0548 (0.6); 1.9647 (0.6); 1.9612 (0.6); 1.9539 (0.8); 1.9471 (0.7); 1.9387 (0.6); 1.9091 (0.6); 1.8986 (0.6); 1.6912 (1.0); 1.6740 (1.9); 1.6556 (2.0); 1.6384 (1.1); 1.5403 (16.0); 0.9579 (3.6); 0.9515 (0.8); 0.9394 (7.3); 0.9329 (1.4); 0.9208 (3.2); 0.9143 (0.6); 0.0080 (0.8); -0.0002 (25.5); -0.0085 (0.9)

I-67: 1H-NMR (400.0 MHz, CDCl3):

δ=7.6150 (0.9); 7.5974 (0.9); 7.2614 (47.3); 7.2555 (1.2); 7.2273 (0.6); 7.2214 (0.5); 7.2145 (3.4); 7.2113 (2.8); 7.2088 (4.0); 7.2053 (2.6); 7.1983 (2.9); 7.1951 (4.3); 7.1894 (3.7); 7.1852 (1.1); 7.1825 (1.0); 7.1793 (0.8); 7.1768 (0.7); 6.9550 (0.9); 6.9492 (1.6); 6.9438 (1.0); 6.9334 (1.7); 6.9276 (3.0); 6.9218 (1.6); 6.9118 (0.9); 6.9059 (1.5); 6.9002 (0.7); 5.2991 (2.5); 5.0658 (1.0); 5.0501 (2.6); 5.0345 (3.6); 5.0188 (2.8); 5.0033 (1.4); 4.9880 (0.7); 4.3750 (0.6); 4.3653 (0.7); 4.3571 (1.1); 4.3417 (1.1); 4.3337 (0.8); 4.3240 (0.6); 4.0107 (2.4); 3.9655 (3.3); 3.9503 (0.6); 3.7506 (1.0); 3.7316 (6.0); 3.7054 (0.7); 3.6865 (4.5); 2.9109 (0.6); 2.9076 (0.6); 2.8996 (1.0); 2.8964 (1.1); 2.8885 (1.1); 2.8850 (1.2); 2.8737 (1.2); 2.8656 (0.6); 2.1506 (0.9); 2.1333 (1.0); 2.1276 (1.0); 2.1155 (1.4); 2.1104 (1.0); 2.0982 (1.4); 2.0927 (1.7); 2.0862 (0.8); 2.0748 (2.1); 2.0625 (1.0); 2.0514 (1.2); 2.0428 (1.3); 2.0199 (0.8); 1.9935 (0.5); 1.9757 (0.8); 1.9604 (0.9); 1.9560 (0.7); 1.9486 (0.7); 1.9449 (1.1); 1.9411 (0.9); 1.9351 (1.0); 1.9297 (2.3); 1.9205 (2.3); 1.9146 (0.9); 1.9055 (1.7); 1.9021 (1.7); 1.8936 (2.1); 1.8914 (2.0); 1.8818 (1.1); 1.8706 (1.1); 1.8568 (1.5); 1.8489 (0.8); 1.8194 (0.6); 1.8066 (0.7); 1.7887 (1.0); 1.7777 (1.0); 1.7667 (0.7); 1.7595 (0.7); 1.5652 (8.9); 1.2595 (15.7); 1.2523 (16.0); 1.2439 (15.6); 1.2366 (15.5); 1.2274 (4.9); 1.2118 (4.6); 0.0080 (0.8); -0.0002 (22.4); -0.0062 (0.6); -0.0085 (0.8)

I-95: 1H-NMR (400.0 MHz, CDCl3):

δ=7.2595 (33.4); 7.2094 (0.6); 7.2037 (0.7); 7.1897 (0.6); 7.1843 (0.6); 3.9601 (0.6); 3.7247 (0.9); 3.6797 (0.7); 1.5368 (7.8); 1.4506 (16.0); 1.4324 (2.1); -0.0002 (12.3)

I-99: 1H-NMR (400.0 MHz, CDCl3):

δ=7.5199 (0.9); 7.4979 (1.1); 7.4804 (1.2); 7.4054 (0.9); 7.3846 (0.9); 7.2611 (134.6); 7.2321 (0.9); 7.2263 (1.2); 7.2201 (1.4); 7.2134 (5.1); 7.2074 (8.0); 7.2042 (5.4); 7.2013 (5.4); 7.1973 (5.3); 7.1941 (6.6); 7.1916 (5.4); 7.1881 (8.0); 7.1820 (4.4); 7.1755 (1.5); 7.1694 (1.1); 6.9970 (0.7); 6.9600 (1.5); 6.9543 (2.4); 6.9484 (1.4); 6.9384 (3.0); 6.9326 (4.9); 6.9269 (2.6); 6.9168 (1.6); 6.9111 (2.5); 6.9053 (1.2); 5.2992 (2.4); 4.4460 (0.6); 4.4316 (0.9); 4.4209 (1.0); 4.4161 (3.4); 4.4050 (4.2); 4.4019 (5.7); 4.3909 (11.5); 4.3877 (5.9); 4.3858 (6.1); 4.3773 (12.9); 4.3710 (8.4); 4.3627 (6.5); 4.3579 (5.7); 4.3473 (1.8); 4.3415 (1.4); 4.3369 (1.1); 4.3339 (1.2); 4.3280 (0.8); 4.3227 (0.8); 4.0008 (2.9); 3.9886 (2.2); 3.9557 (4.2); 3.9429 (3.1); 3.7544 (0.6); 3.7418 (6.6); 3.7386 (9.5); 3.7353 (8.1); 3.7214 (10.6); 3.7147 (10.4); 3.7070 (6.6); 3.7006 (16.0); 3.6968 (6.1); 3.6936 (7.4); 3.6866 (9.4); 3.6792 (1.0); 3.6764 (1.0); 3.6742 (1.1); 3.6625 (0.8); 3.0233 (0.6); 3.0091 (0.9); 3.0007 (1.8); 2.9865 (2.2); 2.9799 (1.9); 2.9745 (1.8); 2.9660 (1.6); 2.9514 (0.8); 2.9443 (0.6); 2.3210 (0.9); 2.3030 (1.0); 2.2987 (1.0); 2.2863 (1.3); 2.2807 (1.0); 2.2682 (1.2); 2.2639 (1.2); 2.2460 (1.1); 2.2170 (1.3); 2.1998 (1.4); 2.1942 (1.3); 2.1821 (1.8); 2.1769 (1.5); 2.1647 (1.8); 2.1592 (1.6); 2.1418 (1.6); 2.1221 (0.9); 2.1091 (1.2); 2.0962 (1.1); 2.0864 (1.3); 2.0812 (1.2); 2.0748 (0.8); 2.0656 (0.6); 2.0581 (1.8); 2.0476 (0.7); 2.0364 (1.3); 2.0201 (2.7); 2.0095 (4.0); 2.0029 (5.1); 1.9951 (5.2); 1.9881 (3.7); 1.9813 (3.6); 1.9729 (2.6); 1.9650 (2.0); 1.9522 (1.3); 1.9377 (1.6); 1.9248 (1.9); 1.9170 (1.5); 1.9047 (1.8); 1.8918 (2.1); 1.8804 (1.2); 1.8715 (1.3); 1.8592 (0.8); 1.8276 (1.1); 1.8100 (0.7); 1.7985 (1.5); 1.7859 (1.3); 1.7118 (0.6); 1.6986 (1.2); 1.6812 (0.8); 1.5560 (10.4); 1.2556 (0.8); 0.0079 (2.0); -0.0002 (67.0); -0.0085 (2.4)

I-137: 1H-NMR (400.0 MHz, CDCl3):

δ=7.2604 (30.4); 7.2128 (1.2); 7.2070 (1.6); 7.2037 (1.0); 7.1967 (1.1); 7.1934 (1.6); 7.1877 (1.4); 7.1810 (0.6); 6.9503 (0.6); 6.9344 (0.6); 6.9286 (1.1); 6.9229 (0.6); 6.9070 (0.5); 4.2798 (1.8); 4.2722 (1.4); 4.2682 (1.9); 4.2640 (1.4); 4.2564 (2.1); 4.0029 (0.9); 3.9577 (1.2); 3.7316 (2.2); 3.6865 (1.7); 3.6091 (2.0); 3.6035 (0.9); 3.5997 (1.3); 3.5972 (2.0); 3.5914 (0.9); 3.5855 (1.8); 3.3980 (1.5); 3.3944 (2.7); 3.3886 (1.2); 3.3826 (16.0); 3.3749 (2.0); 2.9794 (0.5); 2.1336 (0.5); 2.0002 (0.5); 1.9814 (0.8); 1.9687 (1.0); 1.9573 (1.0); 1.9450 (0.6); 1.9075 (0.6); 1.5521 (0.6); 0.0080 (0.6); -0.0002 (18.0); -0.0085 (0.6)

I-151: 1H-NMR (400.0 MHz, CDCl3):

δ=7.5933 (0.7); 7.5755 (0.7); 7.2602 (81.8); 7.2253 (0.5); 7.2189 (0.5); 7.2124 (2.7); 7.2066 (3.3); 7.2031 (2.2); 7.1963 (2.6); 7.1931 (3.4); 7.1873 (3.1); 7.1805 (1.3); 7.1748 (0.6); 6.9554 (0.7); 6.9496 (1.4); 6.9439 (0.9); 6.9337 (1.5); 6.9280 (2.5); 6.9223 (1.3); 6.9122 (0.8); 6.9064 (1.2); 6.9007 (0.6); 4.3874 (0.5); 4.3671 (0.9); 4.3621 (0.8); 4.3562 (0.9); 4.2978 (0.7); 4.2858 (0.8); 4.2756 (2.8); 4.2660 (2.9); 4.2627 (2.8); 4.2612 (2.7); 4.2518 (3.0); 4.2382 (0.7); 4.2262 (0.7); 4.0025 (1.8); 3.9906 (0.6); 3.9572 (2.5); 3.9455 (0.8); 3.7505 (0.6); 3.7314 (4.9); 3.7060 (0.6); 3.6864 (3.5); 3.6650 (0.7); 3.6528 (0.8); 3.6462 (3.9); 3.6395 (1.7); 3.6355 (3.2); 3.6326 (3.3); 3.6290 (1.7); 3.6221 (3.4); 3.5583 (0.8); 3.5547 (2.0); 3.5480 (1.4); 3.5407 (1.3); 3.5371 (6.3); 3.5305 (1.6); 3.5284 (1.2); 3.5196 (6.7); 3.5131 (0.8); 3.5108 (1.0); 3.5021 (2.3); 3.0006 (0.6); 2.9892 (0.9); 2.9779 (1.1); 2.9664 (1.0); 2.9564 (0.7); 2.1835 (0.8); 2.1659 (0.9); 2.1606 (0.8); 2.1484 (1.1); 2.1432 (0.8); 2.1310 (1.1); 2.1257 (1.0); 2.1084 (1.0); 2.0994 (0.7); 2.0866 (0.8); 2.0754 (0.9); 2.0639 (0.8); 2.0598 (0.7); 2.0351 (1.0); 2.0167 (0.5); 1.9998 (1.1); 1.9809 (1.8); 1.9681 (2.2); 1.9567 (2.0); 1.9444 (1.2); 1.9263 (0.8); 1.9157 (0.8); 1.9036 (1.2); 1.8942 (0.8); 1.8809 (0.8); 1.8681 (0.9); 1.8592 (0.6); 1.8153 (0.6); 1.8022 (0.6); 1.7865 (0.9); 1.7740 (0.9); 1.2300 (1.9); 1.2238 (8.0); 1.2182 (1.4); 1.2125 (3.6); 1.2063 (16.0); 1.2007 (2.4); 1.1950 (1.9); 1.1888 (7.7); 1.1832 (1.1); 0.0079 (1.6); -0.0002 (48.8); -0.0085 (1.7)

I-171: 1H-NMR (400.0 MHz, CDCl3):

δ=7.3392 (1.5); 7.3322 (1.0); 7.3230 (1.9); 7.3195 (1.9); 7.3162 (1.4); 7.3126 (1.3); 7.3035 (1.5); 7.2966 (0.9); 7.2607 (36.2); 3.9878 (1.0); 3.9791 (0.7); 3.9429 (1.3); 3.9339 (1.0); 3.7382 (9.0); 3.7250 (16.0); 3.7091 (2.8); 3.6900 (1.3); 3.6782 (1.6); 3.6642 (2.0); 2.9540 (0.5); 2.9458 (0.6); 2.9427 (0.7); 2.9314 (0.7); 2.1401 (0.6); 2.1229 (0.6); 2.1171 (0.5); 2.1002 (0.6); 2.0899 (0.5); 2.0603 (0.6); 1.9950 (0.6); 1.9879 (0.7); 1.9833 (0.6); 1.9772 (1.0); 1.9728 (1.1); 1.9571 (1.0); 1.9490 (0.9); 1.9427 (0.9); 1.9345 (0.7); 1.9052 (0.6); 1.8985 (0.6); 1.8871 (0.6); 1.8634 (0.6); 1.5512 (4.8); 0.8819 (0.7); -0.0002 (14.1); -0.0085 (0.5)

I-185: 1H-NMR (400.0 MHz, CDCl3):

δ=7.6384 (0.7); 7.6190 (0.7); 7.5204 (0.7); 7.5034 (0.8); 7.4900 (0.7); 7.3448 (0.6); 7.3386 (3.0); 7.3322 (3.1); 7.3225 (3.7); 7.3188 (4.0); 7.3161 (4.1); 7.3125 (4.0); 7.3029 (3.0); 7.2965 (3.0); 7.2617 (65.0); 4.4100 (0.7); 4.3976 (0.7); 4.3828 (0.7); 4.3775 (0.7); 4.3649 (0.9); 4.3509 (0.8); 4.3416 (0.6); 4.2198 (2.2); 4.2096 (0.8); 4.2020 (7.2); 4.1884 (4.4); 4.1866 (5.0); 4.1842 (7.6); 4.1705 (4.4); 4.1689 (4.4); 4.1664 (3.1); 4.1526 (1.5); 4.1511 (1.5); 4.1377 (0.7); 4.1311 (0.7); 4.1198 (0.7); 3.9916 (1.7); 3.9807 (1.9); 3.9467 (2.3); 3.9358 (2.6); 3.7240 (0.7); 3.7094 (4.4); 3.7066 (4.4); 3.6644 (3.2); 3.6617 (3.2); 2.9492 (0.7); 2.9457 (0.7); 2.9343 (1.2); 2.9264 (1.3); 2.9227 (1.5); 2.9119 (1.7); 2.9005 (1.1); 2.8911 (0.7); 2.2720 (0.7); 2.2542 (0.8); 2.2496 (0.7); 2.2373 (0.9); 2.2319 (0.8); 2.2195 (1.0); 2.2150 (0.9); 2.1972 (0.8); 2.1636 (0.7); 2.1464 (0.8); 2.1405 (0.8); 2.1287 (1.0); 2.1235 (0.8); 2.1114 (1.0); 2.1056 (1.2); 2.0886 (1.7); 2.0778 (0.7); 2.0660 (0.8); 2.0589 (1.0); 2.0542 (0.6); 2.0352 (0.8); 2.0049 (0.6); 2.0006 (0.8); 1.9911 (1.7); 1.9794 (1.4); 1.9717 (3.6); 1.9657 (2.2); 1.9620 (2.1); 1.9509 (2.6); 1.9409 (2.0); 1.9370 (2.0); 1.9271 (1.7); 1.9113 (1.3); 1.8989 (1.3); 1.8898 (1.2); 1.8767 (1.7); 1.8645 (1.4); 1.8529 (0.9); 1.8418 (0.9); 1.8277 (0.6); 1.8235 (0.6); 1.8103 (0.6); 1.7922 (0.7); 1.7797 (0.8); 1.7661 (0.5); 1.6950 (0.5); 1.6811 (0.9); 1.6647 (0.6); 1.5698 (16.0); 1.5286 (1.1); 1.3173 (0.6); 1.3068 (7.4); 1.2995 (1.5); 1.2932 (8.1); 1.2890 (15.5); 1.2815 (1.4); 1.2754 (15.6); 1.2711 (8.1); 1.2659 (1.7); 1.2603 (2.6); 1.2576 (7.6); 1.2479 (1.8); 1.2427 (0.8); 1.2300 (0.8); 0.0080 (0.6); -0.0002 (23.4); -0.0085 (1.0)

I-255: 1H-NMR (400.0 MHz, CDCl3):

δ=7.5192 (0.6); 7.5002 (0.6); 7.4806 (0.6); 7.4031 (0.6); 7.3849 (0.6); 7.3370 (2.6); 7.3308 (2.8); 7.3208 (3.2); 7.3172 (3.5); 7.3148 (3.8); 7.3114 (3.4); 7.3014 (2.5); 7.2954 (2.7); 7.2810 (0.5); 7.2604 (79.7); 4.4318 (0.6); 4.4221 (0.6); 4.4163 (2.0); 4.4025 (3.8); 4.3901 (4.7); 4.3877 (4.2); 4.3788 (4.7); 4.3766 (3.5); 4.3727 (3.9); 4.3642 (2.8); 4.3594 (2.7); 4.3517 (0.9); 4.3465 (1.0); 4.3379 (0.5); 4.3342 (0.6); 3.9865 (1.6); 3.9749 (1.6); 3.9413 (2.2); 3.9300 (2.2); 3.7346 (3.8); 3.7208 (6.4); 3.7158 (5.4); 3.7134 (4.9); 3.7107 (4.7); 3.7067 (4.0); 3.7020 (7.6); 3.6878 (4.0); 3.6800 (0.7); 3.6741 (0.8); 3.6682 (3.0); 3.6658 (3.0); 3.0127 (0.5); 3.0039 (1.1); 2.9907 (1.3); 2.9826 (1.2); 2.9693 (1.0); 2.9611 (0.6); 2.3162 (0.6); 2.2981 (0.7); 2.2940 (0.7); 2.2813 (0.8); 2.2760 (0.7); 2.2633 (0.8); 2.2593 (0.8); 2.2411 (0.7); 2.2126 (0.6); 2.1952 (0.7); 2.1897 (0.6); 2.1776 (0.9); 2.1724 (0.7); 2.1601 (0.9); 2.1547 (0.9); 2.1372 (0.8); 2.1127 (0.7); 2.1001 (0.6); 2.0899 (0.8); 2.0847 (0.6); 2.0614 (0.8); 2.0299 (0.8); 2.0235 (1.5); 2.0156 (2.0); 2.0103 (2.4); 2.0017 (3.2); 1.9947 (2.3); 1.9900 (2.1); 1.9773 (1.9); 1.9718 (1.1); 1.9676 (1.1); 1.9617 (0.9); 1.9509 (0.7); 1.9350 (0.8); 1.9250 (1.0); 1.9168 (1.0); 1.9047 (1.2); 1.8922 (1.2); 1.8818 (0.8); 1.8716 (0.9); 1.8276 (0.5); 1.7985 (0.8); 1.7876 (0.6); 1.7108 (0.5); 1.6963 (0.8); 1.5457 (16.0); 0.0079 (1.1); -0.0002 (29.6); -0.0084 (1.1)

I-269: 1H-NMR (400.0 MHz, CDCl3):

δ=7.5183 (0.6); 7.3334 (1.3); 7.3280 (1.0); 7.3172 (1.6); 7.3135 (1.8); 7.2977 (1.2); 7.2923 (0.9); 7.2594 (108.2); 6.9954 (0.6); 4.5321 (0.9); 4.5106 (1.8); 4.5081 (1.4); 4.4896 (1.7); 4.4870 (1.5); 4.4685 (0.6); 4.4662 (0.5); 3.9762 (0.7); 3.9309 (1.0); 3.7132 (1.6); 3.6683 (1.2); 2.0440 (0.8); 2.0276 (0.8); 2.0122 (0.8); 2.0020 (0.8); 1.5323 (16.0); 0.0080 (1.4); -0.0002 (40.0); -0.0085 (1.3)

I-339: 1H-NMR (400.0 MHz, CDCl3):

δ=7.5874 (0.5); 7.4866 (0.6); 7.4672 (0.6); 7.4462 (0.8); 7.4422 (1.0); 7.4351 (1.9); 7.4319 (2.2); 7.4267 (2.9); 7.4137 (3.5); 7.4081 (5.3); 7.4046 (5.0); 7.4013 (4.7); 7.3974 (5.4); 7.3853 (0.9); 7.3817 (0.8); 7.2613 (55.7); 7.2053 (0.7); 7.2015 (1.0); 7.1957 (1.1); 7.1915 (0.8); 7.1844 (1.3); 7.1817 (1.3); 7.1780 (1.8); 7.1752 (1.2); 7.1728 (1.4); 7.1675 (1.2); 7.1603 (1.1); 7.1548 (0.6); 4.4012 (0.6); 4.3879 (0.7); 4.3795 (0.6); 4.3677 (0.6); 4.3589 (0.7); 4.3475 (0.6); 4.2199 (2.0); 4.2020 (7.2); 4.1958 (0.7); 4.1842 (9.8); 4.1780 (0.8); 4.1662 (5.9); 4.1481 (2.0); 4.1344 (0.7); 4.1302 (0.7); 4.1166 (0.7); 4.0269 (1.2); 4.0139 (1.5); 3.9812 (1.7); 3.9688 (2.2); 3.7866 (0.6); 3.7735 (4.1); 3.7696 (3.7); 3.7285 (2.9); 3.7246 (2.6); 2.9318 (0.6); 2.9241 (0.9); 2.9093 (1.3); 2.9016 (1.0); 2.8881 (0.9); 2.2875 (0.6); 2.2697 (0.7); 2.2652 (0.6); 2.2529 (0.8); 2.2474 (0.6); 2.2350 (0.8); 2.2307 (0.8); 2.2129 (0.7); 2.1801 (0.6); 2.1626 (0.6); 2.1571 (0.6); 2.1452 (0.7); 2.1399 (0.6); 2.1278 (0.7); 2.1223 (0.7); 2.1051 (0.6); 2.0737 (0.6); 2.0629 (0.5); 2.0504 (0.5); 2.0448 (0.8); 2.0211 (0.7); 2.0092 (0.5); 2.0026 (0.5); 1.9917 (1.1); 1.9832 (1.4); 1.9798 (1.9); 1.9772 (2.0); 1.9720 (2.1); 1.9683 (2.5); 1.9643 (3.3); 1.9565 (1.5); 1.9534 (1.5); 1.9500 (1.6); 1.9467 (1.8); 1.9376 (1.3); 1.9282 (0.6); 1.9244 (0.6); 1.9210 (0.7); 1.8994 (0.6); 1.8861 (0.9); 1.8768 (0.6); 1.8707 (1.0); 1.8565 (0.9); 1.8369 (0.8); 1.7854 (0.5); 1.7736 (0.6); 1.6768 (0.7); 1.6612 (0.5); 1.5662 (12.5); 1.3067 (6.8); 1.2888 (16.0); 1.2783 (1.1); 1.2700 (14.3); 1.2633 (1.3); 1.2605 (1.7); 1.2520 (5.7); 1.2454 (1.7); 1.2427 (0.8); 1.2276 (0.7); 0.0080 (0.9); -0.0002 (26.1); -0.0085 (0.8)

I-409: 1H-NMR (400.0 MHz, CDCl3):

δ=7.5216 (0.8); 7.4983 (0.6); 7.4519 (0.8); 7.4440 (1.0); 7.4373 (2.0); 7.4334 (3.8); 7.4273 (1.8); 7.4185 (3.2); 7.4127 (4.3); 7.4062 (4.4); 7.4030 (6.6); 7.3922 (0.9); 7.3868 (0.9); 7.3831 (0.9); 7.2627 (80.3); 7.2119 (0.8); 7.2080 (0.8); 7.2050 (1.1); 7.2028 (1.0); 7.1988 (0.7); 7.1907 (1.3); 7.1886 (1.2); 7.1853 (1.6); 7.1818 (1.5); 7.1765 (0.7); 7.1730 (0.9); 7.1661 (0.9); 5.3022 (3.9); 4.5725 (0.8); 4.5581 (1.0); 4.5562 (0.6); 4.5434 (0.9); 4.4166 (1.8); 4.4064 (2.3); 4.4026 (3.4); 4.3924 (4.0); 4.3885 (3.9); 4.3839 (2.4); 4.3786 (2.7); 4.3751 (3.9); 4.3693 (3.2); 4.3605 (2.5); 4.3561 (2.2); 4.3493 (1.3); 4.3396 (0.5); 4.3338 (0.6); 4.0258 (1.2); 4.0117 (1.2); 3.9805 (1.7); 3.9664 (1.8); 3.7942 (1.4); 3.7821 (4.0); 3.7786 (4.1); 3.7650 (0.9); 3.7392 (4.3); 3.7374 (3.3); 3.7336 (2.7); 3.7253 (5.6); 3.7147 (3.8); 3.7112 (3.2); 3.7057 (1.0); 3.7005 (5.2); 3.6950 (0.8); 3.6915 (1.0); 3.6867 (3.1); 3.6775 (0.8); 3.0044 (0.5); 2.9961 (0.8); 2.9922 (0.7); 2.9825 (1.1); 2.9759 (1.0); 2.9692 (0.7); 2.9618 (0.8); 2.3289 (0.5); 2.3109 (0.6); 2.3066 (0.6); 2.2942 (0.7); 2.2887 (0.6); 2.2761 (0.7); 2.2722 (0.7); 2.2541 (0.6); 2.2053 (0.6); 2.2000 (0.6); 2.1878 (0.7); 2.1827 (0.6); 2.1704 (0.7); 2.1651 (0.6); 2.1477 (0.6); 2.0779 (0.5); 2.0548 (0.8); 2.0376 (0.5); 2.0307 (0.8); 2.0212 (1.5); 2.0166 (1.8); 2.0105 (2.2); 2.0014 (2.5); 1.9979 (2.4); 1.9939 (2.1); 1.9904 (1.6); 1.9847 (1.8); 1.9763 (1.0); 1.9669 (0.7); 1.9488 (0.5); 1.9304 (0.6); 1.9176 (1.0); 1.9025 (1.0); 1.8832 (0.8); 1.8688 (0.8); 1.7971 (0.6); 1.7847 (0.5); 1.6954 (0.7); 1.5658 (16.0); 0.0080 (1.0); -0.0002 (30.2); -0.0085 (1.1)

I-471: 1H-NMR (400.0 MHz, CDCl3):

δ=7.5991 (0.7); 7.5919 (0.9); 7.5863 (1.0); 7.5804 (0.8); 7.5727 (1.1); 7.5671 (1.2); 7.5594 (0.7); 7.5535 (0.6); 7.5478 (0.7); 7.5407 (0.6); 7.3734 (0.6); 7.3684 (0.7); 7.3632 (0.8); 7.3593 (0.8); 7.3575 (0.8); 7.3520 (1.0); 7.3483 (1.0); 7.3430 (1.0); 7.3380 (0.9); 7.2698 (1.1); 7.2617 (29.8); 7.2497 (1.4); 7.2468 (1.2); 7.2255 (1.2); 7.2047 (0.6); 4.3833 (0.5); 4.3655 (0.6); 4.0070 (0.9); 3.9975 (1.0); 3.9622 (1.3); 3.9526 (1.5); 3.8066 (0.6); 3.7634 (0.7); 3.7440 (3.2); 3.7372 (16.0); 3.7219 (15.0); 3.6995 (2.2); 3.6892 (1.5); 3.6760 (1.5); 3.1912 (0.6); 3.1479 (0.5); 2.9483 (0.6); 2.9340 (0.8); 2.9259 (0.8); 2.9125 (0.6); 2.2563 (0.5); 2.2385 (0.5); 2.2341 (0.5); 2.1498 (0.5); 2.1325 (0.5); 2.0522 (0.6); 2.0283 (0.5); 1.9917 (0.9); 1.9862 (1.5); 1.9800 (1.2); 1.9755 (1.7); 1.9728 (1.9); 1.9653 (1.4); 1.9555 (1.1); 1.9528 (1.0); 1.9483 (0.9); 1.9445 (0.7); 1.9406 (0.8); 1.8883 (0.8); 1.8736 (0.7); 1.8535 (0.6); 1.8399 (0.6); 1.7057 (3.6); 1.5672 (3.0); -0.0002 (11.8)

I-477: 1H-NMR (400.0 MHz, CDCl3):

δ=7.5976 (1.1); 7.5908 (1.4); 7.5852 (1.2); 7.5790 (0.9); 7.5715 (1.6); 7.5659 (1.8); 7.5583 (1.2); 7.5521 (0.9); 7.5453 (1.2); 7.5396 (1.1); 7.4904 (0.6); 7.4713 (0.6); 7.3714 (0.9); 7.3677 (1.1); 7.3662 (1.1); 7.3610 (1.3); 7.3573 (1.2); 7.3554 (1.3); 7.3517 (1.3); 7.3499 (1.5); 7.3463 (1.7); 7.3448 (1.6); 7.3410 (1.6); 7.3396 (1.6); 7.3359 (1.5); 7.3308 (0.8); 7.2677 (1.7); 7.2613 (53.0); 7.2480 (2.3); 7.2454 (2.0); 7.2256 (1.9); 7.2238 (2.0); 7.2038 (0.9); 4.4030 (0.6); 4.3898 (0.8); 4.3780 (0.6); 4.3751 (0.6); 4.3700 (0.7); 4.3648 (0.5); 4.3595 (0.7); 4.3533 (0.5); 4.3476 (0.6); 4.2191 (2.2); 4.2013 (7.4); 4.1835 (8.7); 4.1661 (4.5); 4.1486 (1.4); 4.1355 (0.6); 4.1303 (0.5); 4.1177 (0.5); 4.0097 (1.2); 3.9981 (1.8); 3.9649 (1.7); 3.9531 (2.5); 3.7429 (4.2); 3.7396 (3.3); 3.6979 (3.0); 3.6947 (2.4); 2.9280 (1.0); 2.9142 (1.2); 2.9057 (1.3); 2.8922 (1.0); 2.8837 (0.5); 2.2822 (0.7); 2.2644 (0.8); 2.2599 (0.8); 2.2476 (0.9); 2.2422 (0.7); 2.2298 (0.9); 2.2254 (0.9); 2.2075 (0.8); 2.1560 (0.6); 2.1504 (0.6); 2.1383 (0.7); 2.1332 (0.6); 2.1212 (0.8); 2.1156 (0.7); 2.0982 (0.8); 2.0796 (0.6); 2.0685 (0.5); 2.0568 (0.5); 2.0499 (0.8); 2.0266 (0.6); 2.0050 (0.9); 1.9866 (1.9); 1.9803 (1.5); 1.9767 (1.9); 1.9718 (2.6); 1.9691 (2.8); 1.9647 (3.0); 1.9565 (2.0); 1.9478 (2.3); 1.9411 (1.4); 1.9359 (1.2); 1.9327 (1.5); 1.9211 (0.8); 1.9022 (0.9); 1.8939 (0.8); 1.8835 (1.2); 1.8703 (1.4); 1.8575 (1.2); 1.8481 (0.9); 1.8360 (1.0); 1.8228 (0.5); 1.7870 (0.5); 1.7742 (0.6); 1.6771 (0.9); 1.6632 (0.6); 1.5640 (8.4); 1.3062 (7.2); 1.2885 (16.0); 1.2780 (1.1); 1.2721 (12.5); 1.2709 (9.8); 1.2641 (1.3); 1.2602 (1.6); 1.2543 (5.3); 1.2462 (1.3); 1.2424 (0.7); 1.2283 (0.6); 0.0079 (0.6); -0.0002 (20.1); -0.0084 (0.9)

I-507: 1H-NMR (400.0 MHz, CDCl3):

δ=7.5968 (1.5); 7.5908 (2.2); 7.5846 (1.9); 7.5782 (1.8); 7.5707 (3.0); 7.5648 (3.3); 7.5577 (1.9); 7.5512 (1.7); 7.5452 (2.3); 7.5390 (1.7); 7.5203 (0.8); 7.4962 (1.2); 7.4772 (1.2); 7.4020 (1.1); 7.3780 (1.8); 7.3728 (2.4); 7.3677 (2.5); 7.3624 (2.5); 7.3587 (2.4); 7.3568 (2.6); 7.3514 (3.1); 7.3476 (3.3); 7.3424 (3.2); 7.3373 (2.8); 7.3322 (1.4); 7.2712 (3.9); 7.2614 (116.7); 7.2515 (5.0); 7.2472 (3.9); 7.2270 (4.0); 7.2115 (0.8); 7.2059 (2.1); 6.9975 (0.6); 5.2993 (0.6); 4.5692 (10.8); 4.5548 (14.0); 4.5400 (12.0); 4.5285 (0.8); 4.4443 (0.6); 4.4302 (0.9); 4.4145 (3.6); 4.4034 (5.3); 4.4004 (6.7); 4.3892 (10.3); 4.3861 (6.3); 4.3832 (5.5); 4.3749 (10.2); 4.3688 (7.4); 4.3599 (6.5); 4.3553 (4.9); 4.3489 (2.4); 4.3390 (1.4); 4.3353 (1.3); 4.3326 (1.5); 4.3253 (0.6); 4.3213 (0.9); 4.0064 (2.9); 3.9938 (2.7); 3.9616 (4.1); 3.9487 (3.9); 3.9304 (3.8); 3.7899 (11.8); 3.7836 (1.4); 3.7750 (14.1); 3.7607 (12.0); 3.7544 (1.6); 3.7482 (7.4); 3.7453 (7.4); 3.7347 (7.2); 3.7208 (11.4); 3.7130 (8.8); 3.7064 (7.0); 3.7031 (7.1); 3.6991 (16.0); 3.6849 (7.2); 3.6735 (1.4); 3.6615 (0.8); 3.0186 (0.6); 3.0046 (1.1); 2.9962 (1.9); 2.9822 (2.5); 2.9759 (2.1); 2.9618 (1.8); 2.9543 (1.0); 2.9466 (0.7); 2.9404 (0.5); 2.3247 (1.2); 2.3067 (1.4); 2.3027 (1.3); 2.2900 (1.5); 2.2847 (1.2); 2.2720 (1.5); 2.2680 (1.4); 2.2499 (1.3); 2.2200 (1.2); 2.2025 (1.4); 2.1973 (1.3); 2.1850 (1.7); 2.1799 (1.4); 2.1676 (1.7); 2.1624 (1.5); 2.1448 (1.4); 2.1186 (0.8); 2.1048 (1.1); 2.0954 (1.0); 2.0926 (1.0); 2.0832 (1.2); 2.0773 (1.2); 2.0615 (0.5); 2.0543 (1.8); 2.0438 (0.7); 2.0367 (1.2); 2.0301 (1.5); 2.0205 (3.2); 2.0168 (3.6); 2.0105 (4.9); 2.0073 (4.6); 2.0032 (5.8); 1.9955 (6.0); 1.9879 (3.4); 1.9822 (3.7); 1.9730 (2.7); 1.9654 (1.8); 1.9513 (1.0); 1.9382 (0.7); 1.9309 (1.3); 1.9192 (1.9); 1.9011 (2.2); 1.8873 (2.0); 1.8674 (1.7); 1.8551 (0.9); 1.8238 (1.1); 1.8053 (0.8); 1.7943 (1.4); 1.7821 (1.2); 1.7696 (0.7); 1.7058 (0.7); 1.6937 (1.4); 1.6772 (1.0); 1.5567 (9.7); 1.2562 (1.0); 0.0080 (1.4); -0.0002 (42.9); -0.0084 (1.6)

I-513: 1H-NMR (400.0 MHz, CDCl3):

δ=7.5934 (3.0); 7.5878 (5.5); 7.5823 (4.0); 7.5746 (3.4); 7.5690 (6.2); 7.5610 (6.4); 7.5554 (4.1); 7.5478 (3.1); 7.5422 (5.3); 7.5368 (3.5); 7.5197 (1.1); 7.3778 (1.8); 7.3729 (3.6); 7.3677 (4.3); 7.3636 (4.7); 7.3569 (4.8); 7.3515 (6.0); 7.3464 (7.0); 7.3422 (7.7); 7.3374 (6.7); 7.3091 (0.6); 7.2728 (7.3); 7.2608 (201.6); 7.2533 (11.9); 7.2491 (9.8); 7.2288 (9.2); 7.2076 (4.2); 6.9967 (1.1); 5.2990 (1.3); 4.6039 (1.0); 4.5828 (3.0); 4.5722 (2.4); 4.5618 (3.1); 4.5511 (7.2); 4.5407 (1.3); 4.5302 (8.2); 4.5245 (3.1); 4.5217 (2.9); 4.5109 (8.4); 4.5035 (8.5); 4.5006 (9.1); 4.4900 (8.0); 4.4824 (9.0); 4.4796 (10.9); 4.4690 (3.1); 4.4613 (3.7); 4.4585 (5.7); 4.4480 (0.6); 4.4374 (1.1); 4.4115 (1.5); 4.3945 (2.8); 4.3799 (3.7); 4.3594 (3.0); 4.3236 (0.5); 3.9985 (5.2); 3.9926 (6.1); 3.9536 (7.5); 3.9476 (8.7); 3.8985 (0.6); 3.7516 (16.0); 3.7434 (3.1); 3.7373 (3.5); 3.7220 (10.4); 3.7066 (11.2); 3.6987 (2.2); 3.6893 (1.3); 3.6759 (0.5); 3.0554 (1.3); 3.0457 (2.2); 3.0386 (3.0); 3.0329 (3.2); 3.0236 (4.6); 3.0184 (3.9); 3.0101 (3.3); 3.0022 (3.3); 2.9876 (1.2); 2.3851 (2.3); 2.3670 (2.8); 2.3632 (2.7); 2.3506 (3.0); 2.3453 (2.7); 2.3324 (3.1); 2.3287 (3.0); 2.3107 (2.6); 2.2707 (1.7); 2.2531 (1.9); 2.2488 (1.7); 2.2359 (2.3); 2.2310 (2.0); 2.2184 (2.3); 2.2136 (2.1); 2.1959 (1.9); 2.1676 (0.6); 2.1365 (1.2); 2.1220 (1.8); 2.1136 (1.6); 2.0990 (1.7); 2.0919 (2.6); 2.0697 (3.4); 2.0634 (3.8); 2.0483 (5.0); 2.0407 (6.9); 2.0349 (9.0); 2.0279 (10.0); 2.0216 (9.2); 2.0161 (10.6); 2.0056 (7.1); 1.9995 (3.7); 1.9897 (3.7); 1.9749 (2.4); 1.9593 (1.9); 1.9479 (1.2); 1.9236 (1.8); 1.9100 (3.0); 1.8996 (3.2); 1.8867 (5.5); 1.8732 (4.1); 1.8653 (2.7); 1.8518 (3.9); 1.8380 (2.0); 1.7954 (1.7); 1.7804 (2.0); 1.7663 (1.7); 1.7125 (1.1); 1.7017 (1.2); 1.6900 (1.6); 1.6794 (2.8); 1.6646 (2.8); 1.6505 (1.4); 1.6390 (0.7); 1.6220 (0.6); 1.5582 (1.4); 1.2561 (0.7); 0.0079 (2.8); -0.0002 (73.3); -0.0084 (2.4)

I-537: 1H-NMR (400.0 MHz, CDCl3):

δ=7.2614 (29.4); 7.2248 (2.6); 7.2219 (2.8); 7.2202 (3.1); 7.1973 (1.1); 7.1924 (1.1); 7.0069 (1.2); 7.0048 (1.1); 6.9838 (1.1); 6.9817 (1.1); 5.2989 (1.1); 4.3781 (0.6); 4.3583 (0.6); 4.3466 (0.5); 4.0044 (1.0); 3.9949 (1.0); 3.9594 (1.4); 3.9499 (1.5); 3.7619 (2.8); 3.7596 (2.8); 3.7367 (16.0); 3.7217 (15.8); 3.7170 (2.7); 3.7147 (2.3); 3.6882 (1.5); 3.6748 (1.5); 2.9404 (0.6); 2.9356 (0.6); 2.9261 (0.9); 2.9186 (0.7); 2.9133 (0.6); 2.9045 (0.6); 2.3856 (15.0); 2.2778 (0.5); 2.2611 (0.5); 2.2433 (0.5); 2.2389 (0.5); 2.1577 (0.5); 2.1404 (0.5); 2.1350 (0.5); 2.0430 (0.6); 2.0201 (0.6); 1.9976 (0.7); 1.9843 (2.0); 1.9786 (1.4); 1.9715 (2.2); 1.9689 (2.1); 1.9623 (1.6); 1.9519 (1.0); 1.9429 (0.8); 1.8810 (0.8); 1.8691 (0.9); 1.8554 (0.6); 1.8465 (0.6); 1.8345 (0.8); 1.5696 (2.8); -0.0002 (11.0)

I-598: 1H-NMR (400.0 MHz, CDCl3):

δ=7.7621 (1.5); 7.7588 (2.8); 7.7554 (1.8); 7.7501 (2.0); 7.7467 (3.2); 7.7433 (1.9); 7.6568 (1.0); 7.6527 (1.7); 7.6507 (1.5); 7.6487 (1.4); 7.6467 (1.8); 7.6427 (1.1); 7.6349 (1.1); 7.6308 (1.8); 7.6288 (1.4); 7.6268 (1.5); 7.6248 (1.8); 7.6208 (1.0); 7.5878 (0.6); 7.5682 (0.6); 7.4809 (1.2); 7.4778 (1.6); 7.4755 (1.8); 7.4729 (1.6); 7.4619 (1.5); 7.4588 (2.0); 7.4564 (2.3); 7.4537 (2.1); 7.4345 (0.7); 7.2622 (28.6); 5.2994 (0.7); 4.4175 (0.8); 4.4043 (1.0); 4.3926 (0.8); 4.1506 (1.1); 4.1328 (3.3); 4.1149 (3.3); 4.0971 (1.1); 4.0440 (1.6); 3.9987 (2.2); 3.7691 (3.3); 3.7665 (3.3); 3.7239 (2.4); 3.7212 (2.5); 3.0289 (0.8); 3.0162 (1.0); 3.0066 (1.0); 2.9939 (0.8); 2.3323 (0.5); 2.3145 (0.6); 2.3102 (0.5); 2.2974 (0.7); 2.2924 (0.6); 2.2796 (0.6); 2.2752 (0.6); 2.2575 (0.5); 2.2097 (0.5); 2.1975 (0.8); 2.1925 (0.6); 2.1800 (0.8); 2.1747 (0.8); 2.1669 (0.6); 2.1570 (0.9); 2.1440 (0.6); 2.1263 (0.5); 2.0698 (0.9); 2.0544 (2.0); 2.0462 (16.0); 2.0402 (2.6); 2.0352 (2.0); 2.0247 (1.8); 2.0154 (1.2); 2.0134 (1.2); 2.0079 (1.2); 2.0011 (1.1); 1.9862 (0.8); 1.9654 (0.6); 1.9520 (0.8); 1.9388 (0.5); 1.9219 (0.8); 1.9181 (0.9); 1.8883 (0.5); 1.8247 (0.6); 1.8120 (0.5); 1.7272 (0.7); 1.2775 (4.2); 1.2596 (8.4); 1.2417 (4.1); 0.0080 (0.5); -0.0002 (16.2); -0.0085 (0.6)

I-604: 1H-NMR (400.6 MHz, CDCl3):

δ=7.7545 (0.9); 7.7509 (2.0); 7.7480 (1.8); 7.7459 (2.2); 7.7423 (1.2); 7.6766 (0.7); 7.6728 (0.8); 7.6692 (1.1); 7.6665 (1.0); 7.6628 (0.9); 7.6590 (0.8); 7.6546 (0.9); 7.6509 (1.0); 7.6481 (1.2); 7.6475 (1.2); 7.6446 (1.0); 7.6410 (0.8); 7.6372 (0.6); 7.4870 (0.7); 7.4851 (0.8); 7.4839 (0.9); 7.4815 (1.0); 7.4790 (0.9); 7.4778 (0.8); 7.4759 (0.7); 7.4680 (0.7); 7.4662 (0.9); 7.4649 (0.9); 7.4627 (1.0); 7.4600 (0.8); 7.4588 (0.8); 7.4570 (0.6); 7.2636 (25.6); 4.0488 (0.8); 4.0419 (0.9); 4.0037 (1.1); 3.9967 (1.2); 3.9577 (0.6); 3.7615 (3.2); 3.7444 (16.0); 3.7290 (16.0); 3.7165 (2.5); 3.6928 (1.1); 3.6800 (1.0); 2.9654 (0.5); 2.9566 (0.5); 2.9536 (0.7); 2.9430 (0.6); 2.9320 (0.5); 2.1164 (0.6); 2.0932 (0.6); 2.0020 (0.8); 1.9878 (0.8); 1.9819 (1.7); 1.9730 (0.8); 1.9705 (0.8); 1.9642 (0.8); 1.9579 (1.0); 1.9474 (0.9); 1.9431 (0.8); 1.9343 (0.7); 1.9142 (0.5); 1.9026 (0.8); 1.8892 (0.6); 1.5813 (2.1); -0.0002 (10.3)

I-610: 1H-NMR (400.0 MHz, CDCl3):

δ=7.7474 (2.0); 7.7437 (4.1); 7.7390 (4.3); 7.7352 (2.4); 7.6684 (1.5); 7.6646 (1.8); 7.6619 (2.2); 7.6584 (2.0); 7.6546 (1.8); 7.6507 (1.6); 7.6465 (1.9); 7.6428 (2.0); 7.6401 (2.1); 7.6365 (1.9); 7.6326 (1.5); 7.6289 (1.2); 7.5387 (0.6); 7.5197 (1.0); 7.4788 (1.7); 7.4764 (2.0); 7.4739 (1.8); 7.4628 (1.4); 7.4598 (1.8); 7.4575 (2.0); 7.4549 (1.6); 7.2609 (70.0); 5.2989 (6.1); 4.4221 (0.6); 4.4101 (0.6); 4.4012 (0.5); 4.3905 (0.7); 4.3730 (0.7); 4.3588 (0.7); 4.3506 (0.5); 4.2244 (1.9); 4.2065 (7.0); 4.1887 (9.3); 4.1709 (5.4); 4.1692 (4.0); 4.1531 (1.3); 4.1512 (1.4); 4.1306 (0.6); 4.1127 (0.5); 4.0478 (1.6); 4.0387 (1.6); 4.0028 (2.1); 3.9934 (2.2); 3.7529 (4.2); 3.7508 (4.3); 3.7078 (3.2); 3.7058 (3.3); 2.9536 (0.6); 2.9431 (1.0); 2.9312 (1.4); 2.9209 (1.4); 2.9096 (1.0); 2.8990 (0.5); 2.2670 (0.6); 2.2491 (0.7); 2.2446 (0.6); 2.2322 (0.8); 2.2269 (0.7); 2.2144 (0.8); 2.2098 (0.8); 2.1920 (0.7); 2.1557 (0.6); 2.1386 (0.7); 2.1326 (0.6); 2.1204 (1.0); 2.1156 (0.9); 2.1033 (1.0); 2.0985 (1.4); 2.0879 (0.7); 2.0804 (0.9); 2.0770 (0.9); 2.0693 (1.0); 2.0432 (2.5); 1.9964 (1.7); 1.9885 (0.8); 1.9754 (2.6); 1.9718 (1.7); 1.9652 (1.7); 1.9617 (1.5); 1.9545 (1.3); 1.9499 (2.5); 1.9400 (1.8); 1.9357 (1.8); 1.9268 (1.5); 1.9202 (1.0); 1.9067 (1.5); 1.8956 (1.3); 1.8850 (1.3); 1.8743 (1.3); 1.8619 (0.8); 1.8509 (0.8); 1.8026 (0.6); 1.7897 (0.7); 1.7003 (0.5); 1.6879 (0.7); 1.6738 (0.5); 1.5511 (16.0); 1.3106 (6.4); 1.3043 (0.5); 1.2955 (7.6); 1.2928 (13.7); 1.2777 (14.2); 1.2750 (7.6); 1.2659 (0.9); 1.2598 (6.8); 1.2482 (0.9); 1.2440 (0.5); 1.2407 (0.6); 0.0080 (1.3); -0.0002 (40.1); -0.0084 (1.5)

I-634: 1H-NMR (400.0 MHz, CDCl3):

δ=7.7421 (0.9); 7.7383 (0.9); 7.7341 (0.9); 7.7307 (0.5); 7.6446 (0.5); 7.4732 (0.6); 7.4542 (0.5); 7.2614 (10.2); 3.7527 (0.8); 3.7450 (0.8); 3.7076 (0.6); 3.6999 (0.6); 1.5620 (0.7); 1.4743 (16.0); 1.4610 (0.6); 1.4519 (15.8); 1.4460 (2.1); 1.4329 (0.9); -0.0002 (5.9)

I-640: 1H-NMR (400.0 MHz, CDCl3):

δ=7.7461 (10.1); 7.7418 (10.4); 7.6702 (2.4); 7.6639 (4.6); 7.6602 (4.4); 7.6576 (3.3); 7.6536 (2.8); 7.6483 (2.9); 7.6420 (4.7); 7.6382 (4.4); 7.6318 (2.5); 7.5206 (2.0); 7.5023 (1.7); 7.4849 (4.4); 7.4816 (4.8); 7.4790 (4.8); 7.4759 (4.2); 7.4658 (4.4); 7.4627 (4.7); 7.4601 (4.4); 7.4569 (3.7); 7.4297 (1.6); 7.4104 (1.6); 7.2616 (57.3); 5.2993 (4.0); 4.4520 (0.9); 4.4381 (1.9); 4.4221 (5.1); 4.4084 (10.5); 4.3945 (14.8); 4.3890 (6.7); 4.3809 (12.8); 4.3747 (8.5); 4.3662 (6.0); 4.3612 (5.4); 4.3514 (2.2); 4.3447 (1.3); 4.3391 (1.1); 4.3359 (1.2); 4.3250 (0.7); 4.0455 (4.0); 4.0357 (3.7); 4.0003 (5.4); 3.9906 (4.9); 3.7724 (1.3); 3.7614 (9.6); 3.7593 (9.4); 3.7386 (8.0); 3.7249 (13.6); 3.7184 (11.8); 3.7141 (9.3); 3.7111 (8.6); 3.7046 (16.0); 3.6906 (8.3); 3.6758 (1.2); 3.6645 (0.6); 3.0201 (1.3); 3.0116 (2.4); 3.0001 (2.8); 2.9907 (2.8); 2.9781 (2.2); 2.9697 (1.3); 2.9567 (0.6); 2.3139 (1.5); 2.2957 (1.6); 2.2918 (1.5); 2.2790 (1.8); 2.2737 (1.6); 2.2609 (1.9); 2.2570 (1.8); 2.2387 (1.5); 2.2085 (1.4); 2.1911 (1.6); 2.1856 (1.5); 2.1733 (2.0); 2.1684 (1.7); 2.1560 (2.1); 2.1505 (2.0); 2.1334 (2.1); 2.1220 (1.5); 2.1094 (1.5); 2.0989 (1.7); 2.0938 (1.5); 2.0872 (1.1); 2.0704 (1.8); 2.0546 (0.9); 2.0486 (0.9); 2.0435 (1.4); 2.0325 (2.4); 2.0260 (3.3); 2.0087 (6.2); 2.0057 (5.8); 2.0029 (6.0); 1.9955 (5.1); 1.9921 (5.8); 1.9795 (4.8); 1.9686 (2.7); 1.9522 (1.4); 1.9445 (1.8); 1.9314 (3.1); 1.9179 (2.9); 1.9059 (2.2); 1.8958 (2.5); 1.8831 (2.4); 1.8693 (1.1); 1.8377 (1.2); 1.8198 (1.0); 1.8082 (1.8); 1.7968 (1.5); 1.7159 (1.1); 1.7011 (1.8); 1.6853 (1.1); 1.5581 (15.0); 1.2587 (1.0); 0.0079 (1.8); -0.0002 (50.0); -0.0085 (1.9)

I-646: 1H-NMR (400.0 MHz, CDCl3):

δ=7.7488 (15.9); 7.7455 (16.0); 7.7422 (6.9); 7.6763 (3.2); 7.6706 (5.9); 7.6667 (5.7); 7.6612 (3.5); 7.6544 (3.7); 7.6487 (6.0); 7.6449 (5.7); 7.6393 (3.2); 7.6306 (0.5); 7.4894 (5.6); 7.4861 (5.9); 7.4833 (5.7); 7.4801 (5.3); 7.4704 (5.8); 7.4671 (6.0); 7.4643 (5.5); 7.4611 (4.8); 7.3541 (2.0); 7.3356 (2.0); 7.2635 (65.0); 7.2469 (2.1); 5.3000 (2.0); 4.6119 (0.8); 4.5909 (2.5); 4.5802 (1.9); 4.5699 (2.6); 4.5592 (5.8); 4.5489 (1.1); 4.5381 (8.2); 4.5340 (3.7); 4.5323 (3.6); 4.5237 (0.8); 4.5170 (8.9); 4.5129 (10.1); 4.5113 (10.0); 4.5064 (2.3); 4.4960 (7.2); 4.4919 (10.3); 4.4903 (10.4); 4.4853 (4.4); 4.4795 (0.9); 4.4750 (2.5); 4.4709 (3.7); 4.4693 (3.9); 4.4643 (3.8); 4.4586 (0.6); 4.4482 (0.5); 4.4432 (1.4); 4.4334 (1.0); 4.4159 (2.1); 4.4010 (2.8); 4.3866 (2.8); 4.3709 (2.2); 4.3588 (1.3); 4.3530 (1.2); 4.3411 (0.5); 4.0411 (6.5); 3.9959 (9.0); 3.7816 (1.2); 3.7722 (12.4); 3.7702 (12.3); 3.7362 (0.9); 3.7270 (9.1); 3.7250 (9.1); 3.0760 (0.8); 3.0696 (1.0); 3.0627 (1.8); 3.0541 (2.8); 3.0487 (2.8); 3.0400 (3.9); 3.0324 (3.1); 3.0266 (3.0); 3.0185 (2.7); 3.0048 (1.1); 2.9975 (0.6); 2.3795 (1.7); 2.3614 (2.0); 2.3575 (2.0); 2.3448 (2.3); 2.3395 (1.9); 2.3267 (2.3); 2.3230 (2.2); 2.3049 (1.8); 2.2647 (1.7); 2.2472 (1.9); 2.2423 (1.8); 2.2297 (2.5); 2.2248 (1.9); 2.2123 (2.3); 2.2074 (2.2); 2.1898 (2.0); 2.1579 (0.9); 2.1533 (1.3); 2.1398 (2.0); 2.1299 (1.7); 2.1164 (1.4); 2.1076 (2.5); 2.0845 (2.4); 2.0735 (1.8); 2.0671 (3.2); 2.0593 (1.9); 2.0561 (2.3); 2.0492 (8.1); 2.0436 (3.5); 2.0359 (6.8); 2.0303 (9.6); 2.0216 (4.8); 2.0169 (5.5); 2.0066 (5.4); 2.0016 (2.8); 1.9921 (3.0); 1.9875 (2.2); 1.9771 (1.7); 1.9729 (2.0); 1.9573 (1.5); 1.9424 (0.9); 1.9336 (1.8); 1.9194 (3.2); 1.9014 (3.6); 1.8869 (3.3); 1.8674 (2.7); 1.8540 (1.4); 1.8270 (1.0); 1.8092 (1.4); 1.7946 (2.0); 1.7828 (1.8); 1.7699 (1.0); 1.7668 (1.1); 1.7192 (0.8); 1.7027 (1.0); 1.6904 (2.0); 1.6739 (1.8); 1.6594 (0.9); 1.6484 (0.5); 1.5795 (3.4); 1.2588 (1.1); 1.2409 (0.5); 0.0079 (1.1); -0.0002 (36.4); -0.0085 (1.4)

I-652: 1H-NMR (400.0 MHz, CDCl3):

δ=7.7460 (2.9); 7.7411 (3.1); 7.6690 (0.7); 7.6617 (1.2); 7.6583 (1.2); 7.6551 (1.0); 7.6515 (1.0); 7.6473 (0.9); 7.6399 (1.3); 7.6363 (1.2); 7.6333 (1.0); 7.6296 (1.0); 7.6199 (0.6); 7.5149 (0.5); 7.4943 (0.7); 7.4768 (1.5); 7.4579 (1.4); 7.2616 (19.9); 4.3780 (0.6); 4.3634 (0.6); 4.3047 (1.6); 4.2949 (1.7); 4.2922 (1.7); 4.2808 (3.4); 4.2728 (1.5); 4.2687 (2.2); 4.2646 (1.4); 4.2570 (2.3); 4.2315 (0.5); 4.0463 (1.2); 4.0385 (1.3); 4.0012 (1.6); 3.9933 (1.8); 3.7524 (2.9); 3.7073 (2.2); 3.6317 (2.4); 3.6267 (0.8); 3.6199 (2.6); 3.6136 (2.8); 3.6082 (3.0); 3.6049 (1.8); 3.6017 (2.4); 3.5962 (1.0); 3.5902 (2.4); 3.3971 (15.8); 3.3867 (16.0); 3.3748 (2.0); 3.0036 (0.8); 2.9929 (0.9); 2.9809 (0.9); 2.9716 (0.7); 2.2676 (0.5); 2.2509 (0.6); 2.2327 (0.6); 2.2288 (0.6); 2.1391 (0.6); 2.1217 (0.7); 2.1163 (0.8); 2.1037 (0.6); 2.0989 (0.7); 2.0930 (0.5); 2.0808 (0.5); 2.0510 (0.6); 2.0048 (1.1); 1.9967 (0.9); 1.9854 (1.8); 1.9762 (1.7); 1.9656 (1.5); 1.9525 (1.4); 1.9403 (0.8); 1.9219 (0.8); 1.9166 (0.8); 1.9048 (0.9); 1.8933 (0.8); 1.8815 (0.8); 1.8705 (0.6); 1.8594 (0.6); 1.7987 (0.5); 1.6878 (0.6); 1.5641 (7.0); 1.2588 (0.5); 0.0078 (0.6); -0.0002 (16.5); -0.0084 (0.6)

I-670: 1H-NMR (400.0 MHz, CDCl3):

δ=7.5595 (3.3); 7.5548 (4.0); 7.5523 (4.1); 7.5476 (4.4); 7.5409 (0.6); 7.5397 (0.6); 7.4668 (1.4); 7.4646 (1.4); 7.4622 (2.3); 7.4600 (2.1); 7.4575 (1.4); 7.4554 (1.1); 7.2608 (30.4); 5.2989 (16.0); 4.0042 (0.5); 3.9958 (0.7); 3.9591 (0.7); 3.9508 (1.0); 3.7397 (13.6); 3.7253 (9.7); 3.6943 (1.6); 3.6895 (1.1); 3.6776 (0.9); 2.9294 (0.5); 1.9930 (0.7); 1.9885 (0.5); 1.9821 (0.7); 1.9759 (1.0); 1.9730 (1.0); 1.9694 (0.9); 1.9626 (0.7); 1.9555 (0.7); 1.9521 (0.7); 1.9441 (0.5); 1.9382 (0.5); 1.8904 (0.6); 1.5544 (4.3); -0.0002 (11.0)

I-706: 1H-NMR (400.0 MHz, CDCl3):

δ=7.5575 (6.2); 7.5528 (7.5); 7.5505 (6.7); 7.5457 (7.1); 7.5406 (1.1); 7.5185 (0.8); 7.4685 (2.6); 7.4672 (2.7); 7.4638 (3.7); 7.4627 (3.9); 7.4593 (2.0); 7.4030 (0.5); 7.2596 (138.6); 7.2096 (0.6); 6.9956 (0.8); 5.2985 (2.6); 4.5688 (2.1); 4.5545 (2.7); 4.5396 (2.3); 4.4165 (1.4); 4.4051 (2.2); 4.4024 (2.6); 4.3910 (3.6); 4.3890 (3.0); 4.3846 (2.2); 4.3756 (3.7); 4.3699 (2.8); 4.3610 (2.6); 4.3567 (1.9); 4.3476 (0.8); 4.3402 (0.5); 4.0030 (1.2); 3.9909 (1.1); 3.9577 (1.6); 3.9457 (1.5); 3.7892 (2.3); 3.7743 (2.7); 3.7600 (2.2); 3.7527 (0.5); 3.7421 (3.1); 3.7395 (3.3); 3.7355 (3.2); 3.7215 (4.6); 3.7149 (3.2); 3.7073 (2.9); 3.7008 (5.2); 3.6971 (2.7); 3.6946 (2.6); 3.6869 (3.2); 3.6734 (0.6); 3.0001 (0.8); 2.9869 (0.8); 2.9790 (0.8); 2.9657 (0.7); 2.2817 (0.6); 2.2636 (0.6); 2.2595 (0.5); 2.1945 (0.5); 2.1769 (0.6); 2.1715 (0.5); 2.1594 (0.6); 2.1539 (0.6); 2.1367 (0.6); 2.1091 (0.5); 2.0865 (0.5); 2.0577 (0.7); 2.0336 (0.5); 2.0263 (0.7); 2.0195 (1.1); 2.0127 (1.2); 2.0078 (1.8); 2.0040 (1.7); 1.9995 (2.3); 1.9924 (2.0); 1.9851 (1.6); 1.9782 (1.2); 1.9710 (1.0); 1.9623 (0.6); 1.9344 (0.6); 1.9217 (0.8); 1.9130 (0.6); 1.9050 (0.8); 1.8897 (0.7); 1.8705 (0.6); 1.7976 (0.5); 1.6942 (0.6); 1.5367 (16.0); 1.2557 (1.0); 1.0792 (0.8); 0.0690 (9.7); 0.0079 (1.8); -0.0002 (52.4); -0.0085 (1.9)

I-712: 1H-NMR (400.0 MHz, CDCl3):

δ=7.5553 (1.9); 7.5505 (2.5); 7.5493 (2.6); 7.5446 (2.6); 7.5183 (0.6); 7.4717 (0.8); 7.4671 (1.3); 7.4625 (0.7); 7.2682 (0.7); 7.2595 (106.4); 7.2497 (1.3); 7.2489 (1.2); 7.2481 (1.2); 7.2465 (1.0); 7.2456 (1.0); 7.2449 (0.9); 7.2441 (0.9); 7.2433 (0.9); 7.2425 (0.8); 7.2417 (0.8); 7.2409 (0.8); 7.2401 (0.8); 7.2385 (0.7); 7.2345 (0.6); 7.2297 (0.6); 7.2281 (0.6); 7.2226 (0.5); 6.9954 (0.6); 4.5507 (0.5); 4.5296 (0.6); 4.5142 (0.6); 4.5079 (0.7); 4.5038 (0.7); 4.4932 (0.6); 4.4868 (0.7); 4.4827 (0.8); 3.9443 (0.5); 3.7454 (1.1); 3.7439 (1.0); 3.7003 (0.8); 2.0420 (0.5); 2.0312 (0.5); 2.0261 (0.7); 2.0229 (0.6); 2.0020 (0.5); 1.5325 (16.0); 0.0079 (1.2); -0.0002 (41.7); -0.0085 (1.6)

I-736: 1H-NMR (400.0 MHz, CDCl3):

δ=7.6954 (1.0); 7.6910 (2.0); 7.6871 (1.9); 7.6835 (2.1); 7.6790 (1.4); 7.5416 (0.7); 7.5382 (1.1); 7.5330 (1.1); 7.5289 (0.8); 7.5224 (1.0); 7.5192 (1.5); 7.5138 (1.4); 7.5098 (1.0); 7.4663 (0.9); 7.4635 (1.4); 7.4609 (1.4); 7.4584 (1.4); 7.4555 (1.0); 7.4462 (1.4); 7.4434 (2.1); 7.4409 (2.0); 7.4383 (2.0); 7.4355 (1.1); 7.3958 (2.1); 7.3762 (2.6); 7.3565 (1.0); 7.2607 (39.3); 4.3782 (0.6); 4.3600 (0.6); 4.3479 (0.5); 4.0175 (0.9); 4.0074 (1.0); 3.9725 (1.3); 3.9624 (1.4); 3.7701 (2.8); 3.7685 (2.8); 3.7380 (15.6); 3.7214 (16.0); 3.6883 (1.1); 3.6748 (1.1); 2.9441 (0.6); 2.9298 (0.9); 2.9225 (0.7); 2.9076 (0.6); 2.2398 (0.5); 2.1527 (0.5); 2.1357 (0.5); 2.1301 (0.5); 2.0476 (0.5); 2.0241 (0.6); 1.9965 (0.8); 1.9841 (1.6); 1.9780 (1.2); 1.9734 (1.6); 1.9700 (2.1); 1.9632 (1.5); 1.9577 (1.2); 1.9551 (1.1); 1.9505 (1.2); 1.9396 (0.8); 1.8843 (0.9); 1.8723 (0.9); 1.8599 (0.6); 1.8500 (0.7); 1.8381 (0.7); 1.5610 (2.2); 0.0080 (0.6); -0.0002 (19.5); -0.0085 (0.8)

I-742: 1H-NMR (400.0 MHz, CDCl3):

δ=7.6944 (1.4); 7.6899 (2.9); 7.6861 (2.6); 7.6829 (3.1); 7.6783 (2.0); 7.6021 (0.5); 7.5829 (0.5); 7.5392 (1.0); 7.5343 (1.5); 7.5309 (1.7); 7.5268 (1.2); 7.5198 (1.7); 7.5165 (2.0); 7.5116 (2.1); 7.5077 (1.5); 7.4851 (0.5); 7.4650 (1.4); 7.4621 (2.1); 7.4596 (2.0); 7.4570 (1.9); 7.4542 (1.2); 7.4448 (1.8); 7.4420 (2.8); 7.4395 (2.7); 7.4368 (2.7); 7.4340 (1.4); 7.3945 (2.6); 7.3888 (0.6); 7.3748 (3.3); 7.3552 (1.3); 7.2605 (76.9); 5.2988 (15.9); 4.4001 (0.5); 4.3874 (0.6); 4.3776 (0.6); 4.3667 (0.6); 4.3580 (0.6); 4.3465 (0.6); 4.2201 (1.6); 4.2022 (6.0); 4.1975 (1.1); 4.1842 (8.0); 4.1798 (1.3); 4.1658 (5.2); 4.1477 (1.8); 4.0237 (1.3); 4.0112 (1.3); 3.9787 (1.8); 3.9662 (1.9); 3.7688 (3.6); 3.7652 (3.7); 3.7237 (2.6); 3.7202 (2.6); 2.9319 (0.5); 2.9238 (0.9); 2.9202 (0.8); 2.9096 (1.2); 2.9018 (1.0); 2.8975 (0.9); 2.8885 (0.9); 2.2849 (0.5); 2.2668 (0.6); 2.2624 (0.6); 2.2502 (0.7); 2.2447 (0.6); 2.2323 (0.7); 2.2280 (0.7); 2.2101 (0.6); 2.1768 (0.5); 2.1595 (0.6); 2.1538 (0.6); 2.1420 (0.7); 2.1367 (0.6); 2.1247 (0.8); 2.1191 (0.8); 2.1019 (0.7); 2.0742 (0.6); 2.0515 (0.5); 2.0453 (0.8); 2.0216 (0.7); 2.0028 (0.5); 1.9902 (1.0); 1.9844 (1.3); 1.9753 (1.8); 1.9705 (2.1); 1.9673 (2.2); 1.9626 (3.0); 1.9580 (1.9); 1.9517 (1.6); 1.9462 (1.8); 1.9355 (1.3); 1.9260 (0.7); 1.9194 (0.7); 1.9070 (0.5); 1.8983 (0.6); 1.8895 (1.0); 1.8701 (1.0); 1.8557 (0.8); 1.8358 (0.8); 1.7853 (0.5); 1.7731 (0.6); 1.6746 (0.7); 1.5526 (11.3); 1.3070 (6.2); 1.2890 (16.0); 1.2781 (0.9); 1.2708 (15.2); 1.2637 (1.4); 1.2603 (1.6); 1.2527 (5.7); 1.2457 (1.1); 1.2425 (0.5); 0.0079 (0.9); -0.0002 (28.8); -0.0085 (1.1)

I-772: 1H-NMR (400.0 MHz, CDCl3):

δ=7.6928 (3.8); 7.6886 (7.4); 7.6847 (7.2); 7.6811 (8.2); 7.6769 (5.3); 7.5403 (2.6); 7.5353 (4.0); 7.5322 (4.6); 7.5280 (3.4); 7.5211 (3.7); 7.5171 (5.4); 7.5131 (6.0); 7.5089 (4.4); 7.4882 (1.6); 7.4675 (3.7); 7.4650 (5.0); 7.4625 (5.5); 7.4598 (4.4); 7.4575 (3.6); 7.4474 (4.9); 7.4449 (6.7); 7.4423 (8.2); 7.4399 (6.8); 7.4373 (4.6); 7.4051 (2.1); 7.3970 (10.8); 7.3853 (2.5); 7.3778 (12.1); 7.3577 (4.9); 7.2610 (140.8); 6.9970 (0.7); 5.2988 (4.2); 4.5688 (3.7); 4.5544 (4.5); 4.5525 (2.9); 4.5396 (4.0); 4.4442 (0.7); 4.4299 (1.2); 4.4143 (5.2); 4.4040 (5.8); 4.4002 (9.9); 4.3899 (10.7); 4.3858 (8.7); 4.3846 (8.5); 4.3798 (7.2); 4.3762 (7.3); 4.3707 (11.4); 4.3651 (9.6); 4.3600 (4.5); 4.3561 (7.0); 4.3519 (6.5); 4.3477 (3.2); 4.3357 (1.8); 4.3316 (1.6); 4.3197 (1.0); 4.0211 (3.3); 4.0070 (3.3); 3.9759 (4.8); 3.9620 (4.9); 3.8979 (0.6); 3.7894 (4.1); 3.7744 (13.5); 3.7710 (10.7); 3.7602 (4.2); 3.7350 (10.3); 3.7293 (7.5); 3.7260 (8.4); 3.7211 (16.0); 3.7103 (11.6); 3.7069 (9.3); 3.7016 (2.8); 3.6960 (15.6); 3.6909 (2.6); 3.6881 (2.2); 3.6822 (9.6); 3.6733 (1.7); 3.6599 (0.9); 3.0000 (1.4); 2.9917 (2.1); 2.9874 (2.0); 2.9777 (2.9); 2.9715 (2.6); 2.9646 (2.2); 2.9574 (2.1); 2.9416 (1.0); 2.3511 (0.8); 2.3268 (1.4); 2.3086 (1.5); 2.3044 (1.7); 2.2922 (1.9); 2.2866 (1.6); 2.2741 (1.8); 2.2699 (1.8); 2.2519 (1.6); 2.2214 (1.4); 2.2039 (1.6); 2.1985 (1.5); 2.1864 (2.0); 2.1812 (1.8); 2.1690 (1.9); 2.1637 (1.9); 2.1462 (1.8); 2.1300 (0.5); 2.1138 (0.9); 2.1007 (1.3); 2.0878 (1.3); 2.0772 (1.4); 2.0735 (1.3); 2.0661 (0.9); 2.0580 (0.8); 2.0509 (2.2); 2.0283 (2.0); 2.0144 (5.2); 2.0085 (6.0); 2.0053 (6.4); 1.9992 (7.8); 1.9937 (6.8); 1.9844 (5.6); 1.9765 (3.8); 1.9703 (2.6); 1.9594 (1.7); 1.9482 (1.4); 1.9283 (1.5); 1.9157 (3.1); 1.9024 (2.8); 1.8811 (2.7); 1.8674 (2.4); 1.8545 (1.2); 1.8218 (1.1); 1.8046 (0.9); 1.7926 (1.8); 1.7810 (1.5); 1.7502 (0.5); 1.7046 (1.0); 1.6927 (2.5); 1.6756 (1.2); 1.6606 (0.6); 1.6463 (0.6); 1.5638 (1.9); 1.2557 (0.7); 0.0080 (2.1); -0.0002 (73.8); -0.0085 (3.1)

I-778: 1H-NMR (400.0 MHz, CDCl3):

δ=7.6898 (0.9); 7.6846 (2.1); 7.6795 (2.5); 7.6746 (1.3); 7.5400 (0.6); 7.5356 (1.2); 7.5325 (1.3); 7.5281 (0.8); 7.5208 (0.9); 7.5181 (1.8); 7.5133 (1.7); 7.5090 (1.1); 7.4679 (1.0); 7.4657 (1.1); 7.4626 (1.0); 7.4504 (1.3); 7.4481 (1.6); 7.4456 (1.9); 7.4433 (1.6); 7.3988 (2.4); 7.3794 (2.8); 7.3594 (1.2); 7.2594 (123.7); 6.9954 (0.7); 4.5798 (0.5); 4.5588 (0.5); 4.5481 (1.4); 4.5336 (0.6); 4.5271 (1.4); 4.5191 (0.6); 4.5127 (1.6); 4.5062 (0.6); 4.4981 (1.5); 4.4932 (1.9); 4.4810 (0.8); 4.4771 (1.6); 4.4724 (1.7); 4.4600 (0.7); 4.4561 (0.6); 4.4514 (0.5); 4.3765 (0.6); 4.3567 (0.6); 4.0125 (0.9); 4.0057 (1.0); 3.9680 (1.3); 3.9606 (1.5); 3.7762 (2.9); 3.7742 (2.6); 3.7311 (2.0); 3.7292 (1.8); 3.0188 (0.8); 3.0046 (0.6); 2.9970 (0.6); 2.3344 (0.5); 2.0665 (0.7); 2.0484 (0.8); 2.0329 (1.8); 2.0241 (1.6); 2.0187 (1.8); 2.0136 (1.4); 2.0066 (1.2); 1.9964 (0.7); 1.9890 (0.7); 1.9059 (0.6); 1.8838 (0.9); 1.8707 (0.8); 1.8492 (0.7); 1.5335 (16.0); 0.0080 (1.5); -0.0002 (46.6); -0.0085 (2.0)

I-802: 1H-NMR (400.0 MHz, CDCl3):

δ=7.4721 (1.5); 7.4681 (1.6); 7.3475 (1.5); 7.3444 (1.5); 7.2710 (2.3); 7.2678 (2.1); 7.2598 (63.5); 5.2984 (2.3); 4.0022 (0.7); 3.9932 (0.7); 3.9572 (1.0); 3.9481 (1.0); 3.7559 (2.1); 3.7372 (9.1); 3.7226 (10.4); 3.7110 (1.6); 3.6879 (0.8); 3.6750 (0.9); 2.9259 (0.6); 2.3692 (10.4); 1.9949 (0.5); 1.9825 (1.2); 1.9765 (0.9); 1.9683 (1.4); 1.9641 (1.1); 1.9488 (0.8); 1.9407 (0.5); 1.8786 (0.5); 1.8672 (0.6); 1.5402 (16.0); 0.0078 (0.9); -0.0002 (24.2); -0.0085 (1.0)

I-862: 1H-NMR (400.0 MHz, CDCl3):

δ=7.9933 (3.7); 7.9894 (6.5); 7.9862 (4.4); 7.9796 (4.7); 7.9766 (7.1); 7.9728 (4.7); 7.8705 (6.2); 7.8506 (7.2); 7.7786 (0.6); 7.7671 (3.1); 7.7624 (5.4); 7.7585 (3.6); 7.7477 (3.9); 7.7449 (6.5); 7.7417 (6.6); 7.7390 (3.7); 7.6103 (0.6); 7.5967 (7.3); 7.5908 (1.6); 7.5770 (13.1); 7.5573 (7.0); 7.5380 (0.6); 7.5189 (1.6); 7.4445 (1.6); 7.4252 (1.7); 7.3101 (1.1); 7.2601 (269.3); 6.9960 (1.5); 5.2985 (16.0); 4.4251 (1.3); 4.4082 (2.3); 4.3953 (2.8); 4.3886 (2.4); 4.3818 (2.2); 4.1494 (0.5); 4.1316 (1.6); 4.1137 (1.7); 4.0959 (0.6); 4.0445 (4.6); 4.0016 (6.2); 3.9992 (6.2); 3.8343 (0.6); 3.7982 (0.7); 3.7842 (13.1); 3.7392 (9.4); 3.0402 (0.7); 3.0260 (1.5); 3.0173 (2.4); 3.0041 (3.0); 2.9952 (2.6); 2.9822 (2.3); 2.9607 (0.6); 2.3368 (1.3); 2.3189 (1.5); 2.3148 (1.4); 2.3021 (1.8); 2.2970 (1.5); 2.2843 (1.7); 2.2800 (1.7); 2.2624 (1.5); 2.2373 (1.4); 2.2196 (1.5); 2.2146 (1.6); 2.2023 (2.0); 2.1973 (1.7); 2.1849 (2.0); 2.1797 (2.1); 2.1625 (2.3); 2.1500 (1.8); 2.1371 (1.4); 2.1278 (1.7); 2.1163 (2.0); 2.1094 (1.7); 2.0861 (0.8); 2.0803 (1.4); 2.0705 (1.5); 2.0557 (4.6); 2.0445 (12.3); 2.0387 (9.1); 2.0322 (5.4); 2.0266 (7.0); 2.0169 (3.6); 2.0085 (3.5); 2.0032 (2.7); 1.9876 (2.2); 1.9697 (1.1); 1.9569 (1.6); 1.9449 (2.5); 1.9324 (2.4); 1.9225 (3.1); 1.9103 (2.9); 1.8982 (1.9); 1.8863 (1.7); 1.8762 (0.9); 1.8418 (0.9); 1.8276 (1.2); 1.8155 (1.6); 1.8027 (1.6); 1.7900 (0.8); 1.7304 (1.1); 1.7181 (1.7); 1.7023 (1.2); 1.6863 (0.8); 1.2768 (2.1); 1.2589 (4.4); 1.2411 (2.1); 0.1462 (0.5); 0.0498 (0.7); 0.0079 (5.0); -0.0002 (147.6); -0.0085 (5.1)

I-868: 1H-NMR (400.0 MHz, CDCl3):

δ=7.9858 (1.0); 7.9814 (2.1); 7.9790 (2.0); 7.9767 (2.3); 7.9723 (1.2); 7.8983 (0.7); 7.8953 (0.9); 7.8940 (0.8); 7.8912 (1.3); 7.8885 (1.0); 7.8841 (0.7); 7.8784 (0.9); 7.8754 (1.1); 7.8741 (1.1); 7.8714 (1.5); 7.8686 (1.1); 7.8673 (0.9); 7.8642 (0.8); 7.7715 (0.8); 7.7670 (1.3); 7.7629 (0.8); 7.7521 (1.0); 7.7481 (1.6); 7.7435 (0.9); 7.5995 (1.9); 7.5798 (2.9); 7.5601 (1.3); 7.2607 (17.9); 4.0564 (0.9); 4.0477 (1.0); 4.0111 (1.2); 4.0027 (1.4); 3.7795 (3.1); 3.7421 (16.0); 3.7344 (2.7); 3.7240 (15.5); 3.6910 (1.0); 3.6768 (1.1); 2.9560 (0.6); 2.9422 (0.7); 2.9337 (0.7); 2.9210 (0.6); 2.1433 (0.5); 2.1261 (0.5); 2.1033 (0.6); 2.0615 (0.5); 1.9947 (1.0); 1.9860 (1.1); 1.9812 (1.3); 1.9780 (1.6); 1.9735 (1.7); 1.9659 (1.1); 1.9562 (1.2); 1.9486 (0.8); 1.9415 (0.8); 1.9100 (0.5); 1.9071 (0.5); 1.8969 (0.8); 1.8848 (0.8); 1.8723 (0.5); 1.8621 (0.6); 1.8500 (0.6); 1.5507 (7.2); 0.0080 (0.8); -0.0002 (22.8); -0.0085 (0.8)

I-874: 1H-NMR (400.0 MHz, CDCl3):

δ=7.9850 (1.4); 7.9806 (3.1); 7.9783 (2.6); 7.9762 (3.3); 7.9719 (1.6); 7.8953 (0.9); 7.8923 (1.2); 7.8881 (1.7); 7.8853 (1.3); 7.8809 (1.0); 7.8754 (1.3); 7.8724 (1.4); 7.8711 (1.4); 7.8683 (2.0); 7.8654 (1.4); 7.8611 (1.1); 7.7701 (1.1); 7.7673 (1.8); 7.7650 (1.8); 7.7614 (1.2); 7.7506 (1.4); 7.7466 (2.2); 7.7420 (1.2); 7.6238 (0.5); 7.5979 (2.3); 7.5782 (3.7); 7.5585 (1.7); 7.5193 (0.9); 7.4994 (0.5); 7.2604 (80.3); 4.4020 (0.5); 4.3868 (0.5); 4.3688 (0.6); 4.3549 (0.5); 4.2240 (1.6); 4.2061 (5.2); 4.1882 (5.9); 4.1849 (3.2); 4.1688 (3.3); 4.1672 (3.0); 4.1496 (1.2); 4.0591 (1.2); 4.0479 (1.3); 4.0136 (1.6); 4.0029 (1.8); 3.7774 (3.4); 3.7745 (3.3); 3.7323 (2.5); 3.7295 (2.4); 2.9339 (0.8); 2.9224 (1.0); 2.9132 (1.0); 2.9018 (0.8); 2.2774 (0.5); 2.2598 (0.6); 2.2553 (0.5); 2.2430 (0.6); 2.2374 (0.5); 2.2251 (0.7); 2.2206 (0.6); 2.2026 (0.5); 2.1307 (0.7); 2.1255 (0.5); 2.1134 (0.7); 2.1078 (0.8); 2.0903 (1.1); 2.0673 (0.5); 2.0602 (0.7); 2.0368 (0.6); 2.0052 (0.7); 1.9908 (1.5); 1.9864 (1.0); 1.9796 (1.0); 1.9723 (2.4); 1.9671 (1.4); 1.9639 (1.4); 1.9568 (1.6); 1.9538 (1.5); 1.9468 (1.4); 1.9407 (1.2); 1.9323 (1.2); 1.9202 (0.6); 1.9131 (0.7); 1.9015 (1.1); 1.8924 (0.8); 1.8826 (1.0); 1.8678 (0.9); 1.8569 (0.6); 1.8473 (0.7); 1.7848 (0.5); 1.6827 (0.6); 1.5462 (2.0); 1.3103 (5.3); 1.2924 (16.0); 1.2746 (15.8); 1.2642 (0.6); 1.2615 (1.0); 1.2567 (5.2); 1.2464 (0.7); 0.0079 (1.5); -0.0002 (46.9); -0.0085 (1.6)

I-904: 1H-NMR (400.0 MHz, CDCl3):

δ=7.9844 (4.0); 7.9800 (8.8); 7.9777 (7.7); 7.9756 (9.3); 7.9713 (4.6); 7.8970 (2.7); 7.8940 (3.5); 7.8926 (3.4); 7.8901 (4.2); 7.8877 (3.8); 7.8864 (3.2); 7.8833 (3.0); 7.8771 (3.6); 7.8740 (4.1); 7.8727 (4.2); 7.8704 (4.8); 7.8678 (4.1); 7.8665 (3.7); 7.8634 (3.1); 7.7715 (3.9); 7.7689 (6.0); 7.7661 (3.9); 7.7521 (4.9); 7.7494 (7.3); 7.7468 (4.3); 7.6105 (0.8); 7.6007 (6.9); 7.5908 (1.4); 7.5811 (11.3); 7.5711 (0.9); 7.5614 (5.0); 7.5198 (0.9); 7.5115 (1.4); 7.4921 (1.4); 7.4192 (1.4); 7.3998 (1.4); 7.2609 (56.7); 4.4499 (0.8); 4.4353 (1.4); 4.4200 (5.1); 4.4061 (8.2); 4.3938 (10.4); 4.3917 (8.2); 4.3901 (7.1); 4.3848 (6.6); 4.3801 (7.2); 4.3763 (10.0); 4.3701 (8.2); 4.3617 (6.3); 4.3569 (5.7); 4.3502 (3.2); 4.3404 (1.2); 4.3385 (1.2); 4.3362 (1.3); 4.3340 (1.3); 4.3270 (0.5); 4.3222 (0.9); 4.0555 (3.4); 4.0441 (3.3); 4.0102 (4.6); 3.9987 (4.6); 3.7945 (1.2); 3.7842 (9.5); 3.7814 (9.1); 3.7493 (1.0); 3.7383 (13.5); 3.7243 (14.1); 3.7147 (10.2); 3.7104 (7.0); 3.7006 (16.0); 3.6949 (1.6); 3.6866 (8.7); 3.6783 (0.9); 3.6750 (1.5); 3.6616 (0.8); 3.0258 (0.8); 3.0204 (0.7); 3.0034 (2.2); 2.9912 (2.6); 2.9832 (2.5); 2.9769 (2.0); 2.9700 (2.0); 2.9537 (0.8); 2.9488 (0.6); 2.3220 (1.4); 2.3039 (1.6); 2.2997 (1.4); 2.2871 (1.8); 2.2817 (1.5); 2.2691 (1.8); 2.2649 (1.7); 2.2469 (1.5); 2.2153 (1.3); 2.1979 (1.5); 2.1924 (1.3); 2.1802 (1.9); 2.1750 (1.5); 2.1629 (1.9); 2.1574 (1.8); 2.1402 (1.8); 2.1327 (0.6); 2.1266 (1.0); 2.1135 (1.4); 2.1039 (1.3); 2.1009 (1.3); 2.0911 (1.6); 2.0856 (1.2); 2.0797 (1.0); 2.0701 (0.7); 2.0626 (1.7); 2.0485 (0.9); 2.0389 (1.4); 2.0311 (1.8); 2.0239 (3.5); 2.0213 (3.5); 2.0175 (3.3); 2.0109 (6.0); 2.0066 (6.1); 2.0031 (6.8); 1.9961 (6.6); 1.9888 (4.0); 1.9831 (3.9); 1.9741 (2.9); 1.9672 (2.2); 1.9553 (1.2); 1.9389 (1.6); 1.9261 (3.0); 1.9139 (2.9); 1.9014 (2.2); 1.8913 (2.4); 1.8789 (2.4); 1.8642 (1.0); 1.8331 (1.1); 1.8197 (0.8); 1.8045 (1.7); 1.7917 (1.4); 1.7847 (0.8); 1.7131 (1.1); 1.6991 (1.8); 1.6823 (1.1); 1.5517 (16.0); 0.0079 (2.4); -0.0002 (74.3); -0.0085 (2.5)

I-916: 1H-NMR (400.0 MHz, CDCl3):

δ=7.9872 (1.0); 7.9830 (2.3); 7.9787 (2.6); 7.9746 (1.4); 7.8958 (0.6); 7.8927 (0.8); 7.8885 (1.2); 7.8854 (1.0); 7.8811 (0.8); 7.8760 (0.8); 7.8729 (1.0); 7.8686 (1.4); 7.8655 (1.0); 7.8612 (0.9); 7.7660 (1.4); 7.7619 (0.9); 7.7508 (1.0); 7.7468 (1.6); 7.5985 (1.9); 7.5919 (0.6); 7.5883 (0.6); 7.5788 (2.9); 7.5591 (1.2); 7.2622 (10.9); 4.3051 (1.4); 4.3039 (1.4); 4.2959 (1.2); 4.2940 (1.4); 4.2916 (1.4); 4.2893 (1.2); 4.2792 (2.1); 4.2708 (1.2); 4.2668 (1.8); 4.2627 (1.2); 4.2551 (2.2); 4.0578 (0.8); 4.0481 (1.0); 4.0127 (1.1); 4.0030 (1.4); 3.7791 (2.4); 3.7765 (2.2); 3.7340 (1.8); 3.7315 (1.6); 3.6319 (2.2); 3.6264 (0.7); 3.6227 (1.3); 3.6201 (2.1); 3.6177 (1.3); 3.6107 (2.2); 3.6084 (2.5); 3.6018 (1.2); 3.5989 (1.8); 3.5936 (0.9); 3.5874 (1.7); 3.3967 (16.0); 3.3891 (2.6); 3.3840 (14.6); 3.3733 (2.0); 2.9969 (0.6); 2.9835 (0.7); 2.9758 (0.6); 2.9714 (0.6); 2.9629 (0.5); 2.1084 (0.6); 2.0011 (0.9); 1.9942 (1.2); 1.9889 (1.3); 1.9828 (1.8); 1.9776 (1.3); 1.9711 (1.4); 1.9637 (0.8); 1.9586 (1.1); 1.9461 (0.6); 1.9091 (0.6); 1.9011 (0.5); 1.8900 (0.6); 1.8759 (0.5); 1.5719 (5.0); -0.0002 (14.1)

I-934: 1H-NMR (400.0 MHz, CDCl3):

δ=7.2800 (2.8); 7.2635 (14.7); 7.1069 (1.4); 5.3021 (3.2); 4.0133 (0.5); 4.0049 (0.6); 3.9683 (0.8); 3.9599 (1.0); 3.7907 (1.4); 3.7879 (1.4); 3.7457 (1.0); 3.7429 (1.2); 3.7366 (8.9); 3.7339 (2.8); 3.7304 (2.2); 3.7228 (8.7); 3.6877 (1.4); 3.6735 (1.2); 2.9169 (0.6); 2.4029 (2.0); 2.3718 (1.5); 2.3405 (16.0); 2.3086 (1.8); 1.9799 (1.0); 1.9741 (1.2); 1.9660 (1.3); 1.9633 (1.3); 1.9575 (1.1); 1.9439 (0.6); 1.8605 (0.8); 1.8266 (0.6); 1.6008 (2.4); -0.0002 (7.9)

I-940: 1H-NMR (400.0 MHz, CDCl3):

δ=7.2774 (3.0); 7.2603 (18.0); 7.1023 (1.4); 4.2161 (0.7); 4.1984 (2.2); 4.1809 (2.8); 4.1641 (2.2); 4.1462 (0.9); 4.1272 (0.5); 4.0137 (0.5); 4.0034 (0.6); 3.9689 (0.8); 3.9581 (0.9); 3.7838 (1.2); 3.7797 (1.3); 3.7389 (0.8); 3.7348 (0.9); 2.8916 (0.6); 2.4020 (2.1); 2.3701 (1.8); 2.3379 (16.0); 2.3056 (1.7); 1.9976 (0.6); 1.9826 (0.8); 1.9712 (1.2); 1.9668 (1.2); 1.9564 (1.5); 1.9504 (1.2); 1.9408 (0.6); 1.8702 (0.5); 1.8572 (0.7); 1.8227 (0.5); 1.5663 (1.3); 1.3048 (2.3); 1.2994 (0.8); 1.2949 (0.8); 1.2867 (5.9); 1.2816 (1.5); 1.2767 (1.6); 1.2686 (6.5); 1.2639 (1.7); 1.2586 (1.6); 1.2506 (2.2); 1.2437 (0.9); 1.2405 (0.5); 0.8818 (1.3); 0.8640 (0.5); -0.0002 (10.0)

I-976: 1H-NMR (400.0 MHz, CDCl3):

δ=7.2774 (3.1); 7.2626 (17.9); 7.1117 (1.4); 5.3019 (0.8); 4.5466 (0.7); 4.5255 (0.8); 4.5100 (1.1); 4.5040 (0.6); 4.4994 (0.5); 4.4888 (1.6); 4.4825 (1.0); 4.4785 (0.7); 4.4676 (1.1); 4.4615 (1.0); 4.4574 (0.6); 4.0077 (0.6); 3.9997 (0.6); 3.9626 (0.9); 3.9547 (0.9); 3.7967 (1.4); 3.7941 (1.4); 3.7517 (1.0); 3.7491 (1.0); 3.0052 (0.6); 2.4034 (2.0); 2.3725 (1.9); 2.3406 (16.0); 2.3087 (1.7); 2.0495 (0.6); 2.0338 (0.8); 2.0256 (1.0); 2.0183 (1.2); 2.0104 (1.0); 2.0042 (0.9); 1.8896 (0.6); 1.8751 (0.6); 1.8554 (0.5); 1.5772 (3.4); -0.0002 (10.0)

I-970: 1H-NMR (400.0 MHz, CDCl3):

δ=7.2796 (2.8); 7.2630 (19.4); 7.1085 (1.3); 5.3022 (1.0); 4.4130 (0.7); 4.4020 (1.0); 4.3988 (1.5); 4.3880 (1.8); 4.3839 (1.8); 4.3798 (1.1); 4.3741 (1.2); 4.3697 (1.8); 4.3653 (1.6); 4.3583 (0.8); 4.3547 (1.1); 4.3518 (1.1); 4.3465 (0.8); 4.0149 (0.5); 4.0017 (0.5); 3.9699 (0.8); 3.9567 (0.8); 3.7939 (1.3); 3.7897 (1.3); 3.7488 (0.9); 3.7448 (0.9); 3.7383 (1.4); 3.7244 (2.5); 3.7169 (0.6); 3.7107 (2.5); 3.7033 (0.6); 3.6971 (2.1); 3.6899 (0.6); 3.6828 (1.2); 2.4036 (1.9); 2.3721 (1.5); 2.3413 (16.0); 2.3095 (1.8); 2.0235 (0.6); 2.0149 (0.7); 2.0081 (1.1); 2.0003 (1.2); 1.9929 (1.4); 1.9858 (1.2); 1.9785 (0.7); 1.8918 (0.7); 1.8572 (0.6); 1.5807 (3.9); -0.0002 (10.4)

I-1000: 1H-NMR (400.0 MHz, CDCl3):

δ=7.7179 (1.1); 7.6640 (1.0); 7.5334 (1.3); 7.2632 (8.1); 5.2994 (16.0); 4.0554 (0.6); 4.0183 (0.7); 4.0103 (0.8); 3.8035 (1.6); 3.7585 (1.2); 3.7399 (7.5); 3.7213 (7.0); 3.6894 (1.0); 3.6740 (0.9); 2.4590 (7.1); 1.9857 (0.9); 1.9808 (0.7); 1.9746 (0.9); 1.9711 (1.0); 1.9646 (0.7); 1.9598 (0.5); 1.9565 (0.5); 1.9522 (0.6); 1.5920 (0.7); -0.0002 (3.0)

I-1102: 1H-NMR (400.0 MHz, CDCl3):

δ=7.2610 (9.6); 6.7980 (1.4); 6.7919 (2.5); 6.7857 (1.6); 6.5583 (0.5); 6.5539 (0.9); 4.3948 (0.8); 4.3861 (1.0); 4.3800 (0.8); 4.3712 (0.9); 4.3654 (0.8); 4.3564 (0.5); 3.8131 (16.0); 3.7729 (0.7); 3.7690 (0.7); 3.7318 (0.8); 3.7279 (0.6); 3.7240 (0.6); 3.7178 (1.3); 3.7084 (0.8); 3.7036 (0.7); 3.6943 (1.2); 3.6801 (0.6); 2.0156 (0.5); 2.0100 (0.5); 1.9998 (0.7); 1.9914 (0.5); 1.5596 (1.3); -0.0002 (4.9)

I-1006: 1H-NMR (400.0 MHz, CDCl3):

δ=7.7172 (2.4); 7.6594 (2.2); 7.5314 (3.0); 7.5188 (0.8); 7.2599 (104.8); 6.9959 (0.6); 4.3907 (0.5); 4.3714 (0.5); 4.3600 (0.6); 4.2214 (1.3); 4.2035 (4.2); 4.1976 (1.8); 4.1857 (4.4); 4.1805 (3.5); 4.1678 (1.7); 4.1631 (3.3); 4.1455 (1.2); 4.0634 (1.1); 4.0526 (1.1); 4.0185 (1.5); 4.0074 (1.6); 3.7993 (2.9); 3.7965 (2.9); 3.7543 (2.1); 3.7516 (2.1); 2.9256 (0.8); 2.9120 (0.9); 2.9035 (0.8); 2.8905 (0.7); 2.4690 (2.7); 2.4573 (16.0); 2.2662 (0.5); 2.2494 (0.5); 2.2315 (0.6); 2.2268 (0.5); 2.1554 (0.6); 2.1497 (0.5); 2.1375 (0.9); 2.1324 (0.6); 2.1203 (0.7); 2.1151 (0.7); 2.0975 (0.6); 2.0485 (0.6); 2.0249 (0.6); 1.9898 (0.9); 1.9845 (1.2); 1.9749 (1.8); 1.9703 (1.8); 1.9677 (1.9); 1.9626 (2.3); 1.9525 (1.6); 1.9450 (1.5); 1.9039 (0.6); 1.8895 (1.1); 1.8762 (0.8); 1.8549 (0.7); 1.8415 (0.6); 1.6761 (0.6); 1.5462 (14.3); 1.3074 (4.6); 1.3045 (1.7); 1.2896 (9.6); 1.2864 (7.4); 1.2717 (5.3); 1.2685 (10.4); 1.2605 (1.6); 1.2556 (1.6); 1.2506 (4.6); 1.2442 (1.0); 0.0079 (1.2); -0.0002 (38.0); -0.0085 (1.4)

I-1108: 1H-NMR (400.0 MHz, CDCl3):

δ=7.2598 (30.6); 6.7958 (1.3); 6.7899 (3.0); 6.7841 (1.9); 6.5615 (0.6); 6.5559 (1.0); 6.5503 (0.5); 4.5084 (0.6); 4.4982 (0.5); 4.4912 (0.6); 4.4874 (0.6); 4.4771 (0.6); 4.4702 (0.5); 3.8931 (0.9); 3.8222 (1.2); 3.8125 (16.0); 3.7751 (0.9); 3.7723 (0.8); 3.7301 (0.6); 3.7272 (0.5); 2.0344 (0.6); 2.0235 (0.6); 2.0185 (0.7); 1.5388 (1.4); -0.0002 (11.9)

I-1036: 1H-NMR (400.0 MHz, CDCl3):

δ=7.7148 (2.4); 7.6629 (2.3); 7.5349 (2.9); 7.2613 (42.6); 5.2989 (9.3); 4.5689 (3.7); 4.5545 (4.6); 4.5527 (2.9); 4.5397 (4.0); 4.4162 (1.4); 4.4052 (1.9); 4.4021 (2.6); 4.3912 (3.2); 4.3878 (2.2); 4.3834 (2.0); 4.3784 (2.6); 4.3697 (3.3); 4.3641 (2.8); 4.3615 (1.6); 4.3550 (1.9); 4.3508 (1.9); 4.3475 (1.4); 4.3330 (0.9); 4.0617 (1.1); 4.0501 (1.0); 4.0166 (1.5); 4.0048 (1.5); 3.8159 (0.6); 3.8110 (0.7); 3.8059 (2.9); 3.8032 (3.0); 3.7896 (4.0); 3.7765 (3.1); 3.7747 (4.8); 3.7604 (5.6); 3.7359 (2.8); 3.7220 (4.5); 3.7083 (4.8); 3.7017 (0.8); 3.6943 (4.5); 3.6873 (1.2); 3.6839 (0.9); 3.6804 (2.8); 3.6732 (1.0); 2.9940 (0.7); 2.9902 (0.6); 2.9815 (0.9); 2.9670 (0.6); 2.9611 (0.6); 2.4595 (16.0); 2.3043 (0.5); 2.2918 (0.6); 2.2863 (0.5); 2.2737 (0.5); 2.2696 (0.5); 2.2015 (0.5); 2.1840 (0.7); 2.1788 (0.5); 2.1666 (0.6); 2.1613 (0.6); 2.1439 (0.5); 2.0541 (0.7); 2.0150 (1.6); 2.0084 (1.8); 2.0000 (2.4); 1.9941 (2.0); 1.9923 (2.0); 1.9852 (1.5); 1.9777 (1.0); 1.9704 (0.8); 1.9601 (0.5); 1.9310 (0.5); 1.9183 (1.1); 1.9060 (1.0); 1.8946 (0.7); 1.8837 (0.9); 1.8715 (0.8); 1.7981 (0.5); 1.6939 (0.5); 1.5588 (3.3); 0.0080 (0.5); -0.0002 (15.6); -0.0085 (0.6)

I-1042: 1H-NMR (400.0 MHz, CDCl3):

δ=7.7142 (2.7); 7.6616 (2.4); 7.5374 (3.0); 7.3241 (0.5); 7.2606 (30.5); 5.2984 (7.4); 4.5839 (0.5); 4.5629 (0.6); 4.5522 (1.3); 4.5313 (1.4); 4.5126 (2.0); 4.5016 (0.6); 4.4918 (3.2); 4.4806 (1.0); 4.4710 (2.4); 4.4684 (2.1); 4.4595 (0.9); 4.4500 (0.7); 4.4473 (0.7); 4.3959 (0.5); 4.3785 (0.7); 4.3615 (0.7); 4.0541 (1.2); 4.0474 (1.1); 4.0090 (1.7); 4.0022 (1.6); 3.8090 (3.4); 3.7640 (2.4); 3.0421 (0.6); 3.0338 (0.6); 3.0292 (0.6); 3.0203 (1.0); 3.0068 (0.7); 3.0002 (0.6); 2.4592 (16.0); 2.3677 (0.5); 2.3640 (0.5); 2.3513 (0.6); 2.3460 (0.6); 2.3331 (0.6); 2.3294 (0.6); 2.2326 (0.6); 2.2151 (0.6); 2.2103 (0.6); 2.1928 (0.5); 2.0919 (0.6); 2.0702 (0.6); 2.0654 (0.9); 2.0496 (0.9); 2.0334 (1.8); 2.0249 (1.9); 2.0186 (2.2); 2.0146 (1.3); 2.0094 (1.3); 2.0028 (0.8); 1.9981 (0.7); 1.9924 (0.6); 1.9879 (0.6); 1.9119 (0.8); 1.8916 (1.0); 1.8770 (1.0); 1.8574 (0.7); 1.5544 (2.6); -0.0002 (11.2)

I-1066: 1H-NMR (400.0 MHz, CDCl3):

δ=7.2604 (15.0); 6.7996 (1.4); 6.7936 (2.5); 6.7875 (1.5); 6.5533 (0.8); 6.5515 (0.8); 5.2986 (0.8); 3.8129 (16.0); 3.7693 (0.7); 3.7663 (0.7); 3.7335 (4.6); 3.7214 (4.9); 1.9738 (0.7); 1.9703 (0.6); 1.9632 (0.5); 1.5512 (2.4); -0.0002 (7.4)

I-1072: 1H-NMR (400.0 MHz, CDCl3):

δ=7.2611 (10.1); 6.7992 (1.4); 6.7931 (2.5); 6.7871 (1.6); 6.5556 (0.5); 6.5521 (0.8); 6.5501 (0.8); 5.2988 (3.6); 4.1975 (1.1); 4.1851 (0.8); 4.1797 (1.1); 4.1674 (0.8); 3.8119 (16.0); 3.7683 (0.7); 3.7638 (0.7); 3.7233 (0.5); 1.9668 (0.7); 1.5651 (1.4); 1.3037 (1.1); 1.2876 (1.5); 1.2860 (2.5); 1.2700 (2.4); 1.2683 (1.5); 1.2522 (1.1); -0.0002 (3.8)

### B. FORMULATION EXAMPLES

### 1. Dusting Products

A dusting product is obtained by mixing 10 parts by weight of a compound of the formula (I) and 90 parts by weight of talc as an inert substance and comminuting the mixture in a hammer mill.

### 2. Dispersible Powder

A readily water-dispersible wettable powder is obtained by mixing 25 parts by weight of a compound of the formula (I), 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.

### 3. Dispersion Concentrate

A readily water-dispersible dispersion concentrate is obtained by mixing 20 parts by weight of a compound of the formula (I), 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to more than 277° C.) and grinding the mixture in a friction ball mill to a fineness of below 5 microns.

### 4. Emulsifiable Concentrate

An emulsifiable concentrate is obtained from 15 parts by weight of a compound of the formula (I), 75 parts by weight of cyclohexanone as a solvent and 10 parts by weight of ethoxylated nonylphenol as an emulsifier.

### 5. Water-Dispersible Granules

Water-dispersible granules are obtained by mixing

```
75 parts by weight of a compound of the formula (I),
10 parts by weight of calcium lignosulfonate,
  5 parts by weight of sodium laurylsulfate,
  3 parts by weight of polyvinyl alcohol and
  7 parts by weight of kaolin,
```

grinding the mixture in a pinned-disk mill, and granulating the powder in a fluidized bed by spray application of water as a granulating liquid.

Water-dispersible granules are also obtained by homogenizing and precomminuting, in a colloid mill,

```
25 parts by weight of a compound of the formula (I),
  5 parts by weight of sodium 2,2′-dinaphthylmethane-6,6′-disulfonate,
  2 parts by weight of sodium oleoylmethyltaurinate,
  1 parts by weight of polyvinyl alcohol,
17 parts by weight of calcium carbonate and
50 parts by weight of water,
```

then grinding the mixture in a bead mill and atomizing and drying the suspension thus obtained in a spray tower by means of a one-phase nozzle.

### C. BIOLOGICAL EXAMPLES

### Test Description

### 1. Pre-Emergence Herbicidal Action and Crop Plant Compatibility

Seeds of monocotyledonous and dicotyledonous weed plants and crop plants are placed in plastic or wood fiber pots and covered with soil. The compounds of the invention, formulated in the form of wettable powders (WP) or as emulsion concentrates (EC), are then applied onto the surface of the covering soil as aqueous suspension or emulsion with addition of 0.5% additive at a water application rate of 6001/ha (converted). After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

In the tables below, the following abbreviations are used:

Undesired plants/weeds:

| | | | |
|---|---|---|---|
| ABUTH: | Abutilon theophrasti | ALOMY: | Alopecurus |
| | | | myosuroides |
| AMARE: | Amaranthus retroflexus | AVEFA: | Avena fatua |
| ECHCG: | Echinochloa crus-galli | HORMU: | Hordeum murinum |
| LOLRI: | Lolium rigidum | MATIN: | Matricaria inodora |
| PHBPU: | Ipomoea purpurea | POLCO: | Polygonum convolvulus |
| SETVI: | Setaria viridis | STEME: | Stellaria media |
| VERPE: | Veronica persica | VIOTR: | Viola tricolor |

TABLE C1
Herbicidal pre-emergence action

| Example | Dosage | Herbicidal action against [%] | | | | | | |
|---|---|---|---|---|---|---|---|---|
| No.: | [g/ha] | ALOMY | AVEFA | ECHCG | LOLRI | SETVI | ABUTH | AMARE |
| I-99 | 80 | 100 | 90 | 90 | 100 | 100 | 80 | 100 |
| I-29 | 80 | 100 | 80 | 90 | 100 | 100 | 70 | 100 |
| I-39 | 80 | 100 | 100 | 100 | 100 | 100 | 70 | 100 |

| Example | Herbicidal action against [%] | | | | | | |
|---|---|---|---|---|---|---|---|
| No.: | MATIN | PHBPU | POLCO | STEME | VIOTR | VERPE | HORMU |
| I-99 | 80 | 90 | n.e. | 100 | 100 | 100 | 100 |
| I-29 | 30 | 80 | 80 | 90 | 90 | 100 | 90 |
| I-39 | 90 | 80 | 100 | n.e. | 100 | n.e. | 90 |

As shown by the results, compounds according to the invention, for example the compounds No. 1-99, I-29 and 1-39, applied by the pre-emergence method, have good to very good activity (80% to 100% herbicidal activity) against harmful plants such as Abufilon theophrasfi, Alopecurus myosuroides, Amaranthus retroflexus, Avena fatua, Echinochloa crus-galli, Hordeum murinum, Lolium rigidum, Matricaria inodora, Ipomoea purpurea, Polygonum convolvulus, Setaria viridis, Stellaria media, Veronica persica and Viola tricolor at an application rate of 0.08 kg of active substance or less per hectare.

### 2. Post-Emergence Herbicidal Action and Crop Plant Compatibility

Seeds of monocotyledonous and dicotyledonous weeds and crop plants are placed in sandy loam in plastic or wood-fiber pots, covered with soil and cultivated in a greenhouse under controlled growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion with addition of 0.5% additive at a water application rate of 6001/ha (converted). After the test plants had been kept in the greenhouse under optimum growth conditions for about 3 weeks, the activity of the preparations is rated visually in comparison to untreated controls. For example, 100% activity=the plants have died, 0% activity=like control plants.

TABLE C2
Herbicidal post-emergence action

| Example | | Herbicidal action against [%] | | | | | | | | |
|---|---|---|---|---|---|---|---|---|---|---|
| No.: | Dosage | ALOMY | AVEFA | ECHCG | LOLRI | AMARE | POLCO | STEME | VERPE | HORMU |
| I-802 | 320 | 90 | 100 | 80 | 90 | 90 | 100 | 90 | 90 | 80 |
| I-42 | 320 | 80 | 90 | 80 | 90 | 80 | 80 | 90 | 80 | 90 |
| I-53 | 320 | 100 | 100 | 100 | 100 | 90 | 100 | 100 | 90 | 100 |
| I-67 | 320 | 100 | 100 | 90 | 100 | 90 | 90 | 100 | 90 | 100 |
| I-11 | 320 | 80 | 90 | 80 | 90 | 80 | 80 | 90 | 80 | 80 |
| I-99 | 320 | 90 | 90 | 90 | 100 | 90 | 80 | 90 | 90 | 90 |
| I-325 | 320 | 90 | 100 | 90 | 100 | 80 | 80 | 90 | 80 | 90 |
| I-29 | 320 | 90 | 90 | 80 | 90 | 80 | 90 | 80 | 80 | 90 |
| I-123 | 320 | 90 | 90 | 80 | 100 | 80 | 90 | 90 | 80 | 90 |
| I-109 | 320 | 90 | 90 | 90 | 80 | 80 | 80 | 90 | 80 | 90 |
| I-543 | 320 | 90 | 100 | 90 | 90 | 100 | 100 | 80 | 90 | 80 |
| I-311 | 320 | 100 | 100 | 100 | 100 | 90 | 100 | 100 | 90 | 100 |
| I-185 | 320 | 90 | 100 | 80 | 90 | 80 | 90 | 80 | 80 | 90 |
| I-95 | 320 | 90 | 100 | 80 | 100 | 80 | 90 | 90 | 80 | 90 |
| I-171 | 320 | 100 | 90 | 80 | 100 | 80 | 80 | 80 | 80 | 80 |
| I-339 | 320 | 100 | 100 | 100 | 100 | 100 | 100 | 100 | 90 | 100 |
| I-409 | 320 | 100 | 100 | 100 | 100 | 90 | 100 | 100 | 90 | 100 |
| I-423 | 320 | 100 | 100 | 100 | 100 | 90 | 90 | 100 | 90 | 100 |

TABLE C3
Herbicidal post-emergence action

| Example | Dosage | Herbicidal action against [%] | | | | | | | | | | |
|---|---|---|---|---|---|---|---|---|---|---|---|---|
| No.: | [g/ha] | ALOMY | AVEFA | DIGSA | ECHCG | LOLRI | SETVI | AMARE | POLCO | VIOTR | VERPE | HORMU |
| I-808 | 320 | 90 | 90 | 90 | 80 | 90 | 90 | 80 | 100 | 80 | 90 | 90 |
| I-537 | 320 | 90 | 90 | 90 | 100 | 90 | 90 | 100 | 90 | 100 | 100 | 80 |
| I-574 | 320 | 100 | 100 | 80 | 90 | 90 | 90 | 100 | 80 | 80 | 90 | 80 |
| I-580 | 320 | 90 | 100 | 90 | 80 | 90 | 90 | 90 | 80 | 100 | 90 | 90 |

TABLE C4
Herbicidal post-emergence action

| Example | Dosage | Herbicidal action against [%] | | | | | |
|---|---|---|---|---|---|---|---|
| No.: | [g/ha] | ALOMY | LOLRI | AMARE | POLCO | VERPE | HORMU |
| I-844 | 320 | 100 | 90 | n.e. | 100 | 90 | 80 |
| I-39 | 320 | 100 | 90 | 80 | 80 | n.e. | 100 |
| I-85 | 320 | 90 | 90 | 80 | n.e. | 80 | 90 |
| I-20 | 320 | 80 | 90 | 80 | n.e. | 90 | 90 |
| I-28 | 320 | 80 | 90 | n.e. | 80 | 80 | 90 |
| I-838 | 320 | 100 | 100 | 80 | 100 | 80 | 90 |

As shown by the results, compounds according to the invention such as, for example, the compounds No. I-802, I-42, I-53, I-67, I-11, I-99, I-325, I-29, I-123, I-109, I-543, I-311, I-185, I-95, I-171, I-339, I-409 and I-423, applied by the post-emergence method, have good to very good herbicidal activity (80% to 100% herbicidal activity) against harmful plants such as Alopecurus myosuroides, Avena fatua, Echinochloa crus-galli, Lolium rigidum, Amaranthus retroflexus, Polygonum convolvulus, Stellaria media, Veronica persica and Hordeum murinum at an application rate of 0.32 kg of active substance or less per hectare.

3. Demonstration of Superiority with Respect to WO 2012/130798

TABLE C3
Herbicidal post-emergence action

| | | | Herbicidal action | |
|---|---|---|---|---|
| | Example | Dosage | against [%] | |
| Document | No. | [g/ha] | ABUTH | STEME |
| Present | I-29 | 80 | 80 | 80 |
| Invention | | | | |
| WO | 6.736 | | 60 | 50 |
| 2012/130798 | | | | |

TABLE C4
Herbicidal pre-emergence action

| | | Dosage | Herbicidal action against [%] | | | | | | |
|---|---|---|---|---|---|---|---|---|---|
| Patent No. | Example No. | [g/ha] | ALOMY | AVEFA | SETVI | AMARE | STEME | VERPE | VIOTR |
| Present | I-29 | 20 | 90 | 70 | 90 | 100 | 90 | 90 | 90 |
| Invention | | | | | | | | | |
| WO | 6.736 | | 70 | 50 | 20 | 0 | 50 | 70 | 0 |
| 2012/ | | | | | | | | | |
| 130798 | | | | | | | | | |

n.e. = no emergence

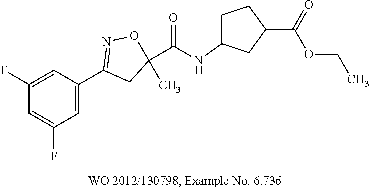 [SMILES: CCOC(=O)C1CCC(NC(=O)C2(C)CC(c3cc(F)cc(F)c3)=NO2)C1]

1. A 3-phenyl-5-trifluoromethylisoxazoline-5-carboxamide of formula (I) 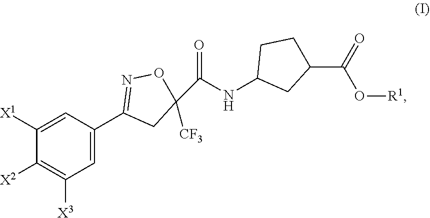
or an agrochemically acceptable salt thereof, wherein
R¹ represents hydrogen,
or
represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, bromine, cyano and (C₁-C₂)-alkoxy;
X¹ and X³ independently of one another represent hydrogen, halogen, hydroxy, cyano, nitro, S(O)ₙR² or CO₂R³,
or
represent (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of halogen and cyano, where X¹ and X³ do not simultaneously represent hydrogen;
X² represents hydrogen, halogen, cyano, nitro, S(O)n R² or CO₂R³,
or
represents (C₁-C₆)-alkyl, (C₁-C₆)-alkoxy, (C₃-C₅)-cycloalkyl, (C₂-C₆)-alkenyl or (C₂-C₆)-alkynyl, each of which is substituted by m radicals from the group consisting of halogen and cyano;
R² represents (C₁-C₆)-alkyl or (C₃-C₆)-cycloalkyl, each of which is substituted by m radicals from the group consisting of halogen and cyano;
R³ represents hydrogen,
or
represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, bromine, cyano and (C₁-C₂)-alkoxy;
m represents the running number 0, 1, 2, 3, 4 or 5; and
n represents the running number 0, 1 or 2.

2. The compound of as claimed in claim 1, where R¹ represents hydrogen, or (C₁-C₆)-alkyl, in each case substituted by m radicals from the group consisting of fluorine, chlorine, cyano and (C₁-C₂)-alkoxy.

3. The compound of as claimed in claim 1, where X¹ and X³ independently of one another represent hydrogen, fluorine, chlorine or cyano, or represent (C₁-C₆)-alkyl or (C₁-C₆)-alkoxy, each of which is substituted by m radicals from the group consisting of fluorine, chlorine and cyano, where X¹ and X³ do not simultaneously represent hydrogen.

4. The compound of as claimed in claim 1, where X² represents hydrogen or halogen.

5. The compound of as claimed in claim 1, where R² represents (C₁-C₆)-alkyl or (C₃-C₆)-cycloalkyl, each of which is substituted by m radicals from the group consisting of fluorine, chlorine and cyano.

6. The compound of as claimed in claim 1, where m is 0, 1, 2 or 3.

7. The compound of as claimed in claim 1,
wherein
R¹ represents hydrogen,
or
represents (C₁-C₆)-alkyl, (C₃-C₆)-cycloalkyl, (C₂-C₄)-alkenyl or (C₂-C₄)-alkynyl, each of which is substituted by m radicals from the group consisting of fluorine, chlorine, cyano and (C₁-C₂)-alkoxy;
X¹ and X³ independently of one another represent hydrogen, fluorine, chlorine or cyano,
or
represent (C₁-C₆)-alkyl or (C₁-C₆)-alkoxy, each of which is substituted by m radicals from the group consisting of fluorine, chlorine and cyano, where X¹ and X³ do not both simultaneously represent hydrogen;
X² represents hydrogen; and
m represents the running number 0, 1, 2 or 3.

8. An herbicidal composition or plant growth-regulating composition, comprising one or more compounds of formula (I) or agrochemically acceptable salts thereof as claimed in claim 1.

9. The herbicidal composition as claimed in claim 8, further comprising a formulation auxiliary.

10. The herbicidal composition as claimed in claim 8, comprising at least one further active compound from the group of insecticides, acaricides, herbicides, fungicides and/or growth regulators.

11. The herbicidal composition as claimed in claim 8, comprising a safener.

12. The herbicidal composition as claimed in claim 11, wherein the safener is selected from the group consisting of mefenpyr-diethyl, cyprosulfamide, isoxadifen-ethyl, cloquintocet-mexyl, benoxacor and dichlormid.

13. A method of controlling one or more unwanted plants, comprising applying an effective amount of at least one compound as claimed in claim 1 or an herbicidal composition thereof to the plants or to a site of unwanted vegetation.

14. A product comprising a compound as claimed in claim 1 or of an herbicidal composition thereof for controlling one or more unwanted plants.

15. The product as claimed in claim 14, wherein the compound is capable of being used for controlling one or more unwanted plants in one or more crops of useful plants.

16. The product as claimed in claim 15, wherein the useful plants are transgenic useful plants.
